(19) United States
(12) Patent Application Publication
(10) Pub. No.: US 2021/0244686 A1
(43) Pub. Date: Aug. 12, 2021

(54) Novel Systems And Methods For The Therapeutic Use of Cannabinoids or Cannabinoid Analogs to Increase The Lipid Order of Cholesterol Containing Cell Membranes

(71) Applicant: The Regents of the University of Colorado a body corporate, Denver, CO (US)

(72) Inventors: Xuedong Liu, Niwot, CO (US); Douglas Chapnick, Boulder, CO (US); William Old, Boulder, CO (US)

(21) Appl. No.: 17/255,214

(22) PCT Filed: Jun. 24, 2019
(86) PCT No.: PCT/US2019/038784

§ 371 Date: Dec. 22, 2020

Related U.S. Application Data
(60) Provisional application No. 62/688,815, filed on Jun. 22, 2018.

(51) Int. Cl. A61K 31/05; A61K 31/352; A61K 31/192; A61K 31/09; A61K 45/06; A61K 31/445; A61K 31/13; A61K 31/27; A61K 31/55; A61P 3/06

(52) U.S. Cl. CPC A61K 31/05; A61K 31/352; A61K 31/192; A61K 31/09; A61P 3/06; A61K 31/445; A61K 31/13; A61K 31/27; A61K 31/55; A61K 45/06

(57) ABSTRACT

The invention includes the use of cannabidiol (CBD), and cannabidiol analogs, as therapeutic compounds or formulations for the increase of lipid order in the cell membranes of a patient. This increase in membrane lipid order may cause downstream therapeutic effects that may ameliorate certain disease conditions such as cardio vascular disease, high-cholesterol and Alzheimer's disease.

### CROSS-REFERENCE TO RELATED APPLICATIONS

This application claims the benefit of and priority to U.S. Provisional Application No. 62/688,815, filed Jun. 22, 2018, which is incorporated herein by reference in its entirety.

### GOVERNMENT INTEREST

This invention was made with government support under DARPA grant W911NF-14-2-0019, and NIH grant 1R01GM113141-01A1. The government has certain rights in the invention.

### TECHNICAL FIELD

The present invention relates to the use of cannabinoids, and cannabinoid analogs, as therapeutic compounds or formulations for the modulation of cholesterol in a patient. The invention includes the use of cannabidiol, and cannabidiol analogs, as therapeutic compounds or formulations for the modulation of cholesterol in a patient which may further be administered in combination with existing cholesterol modulating pharmaceutical compounds. Certain aspects of the invention may include methods of treating lipid-order conditions, such as Alzheimer's disease, as well as activating enhanced T-cell immune responses among other.

### BACKGROUND

The non-psychoactive cannabinoid, cannabidiol (CBD), was recently approved by the FDA for treatment of Lennox-Gastaut and Dravet syndrome (Commissioner), and has been proposed as a treatment for nephrotoxicity, colitis, cancer, neuroinflammation, cardiomyopathies, and diabetic complications. Although preclinical data for several other therapeutic applications of CBD exist, relatively little is known about the biochemical mechanism of action of CBD in cells, tissues, and organs. Indeed, its molecular mechanism of action (MoA) is not well understood relative to its psychoactive structural isomer tetrahydrocannabinol (THC). The molecular targets responsible for the proposed pharmacological effects of CBD have also remained largely unknown. Proposed targets include voltage-dependent anion channel 1 (VDAC1), G protein-coupled receptor 55, and CaV3.x, but substantial evidence is outstanding to significantly support the claims that the observed clinical therapeutic effects of CBD for epilepsy or other diseases originates from these targets.

One consensus across the field is that CBD does not target CB1 or CB2, as is the case for THC. Thus, the accumulating clinical data to support the therapeutic potential of CBD has outpaced the efforts to understand the molecular mechanisms that underlay these effects, in part due to barriers imposed by the classification of CBD as a schedule I drug under the US Controlled Substances Act. A commercial formulation of CBD, Epidiolex®, was recently classified as a schedule V drug, facilitating research efforts to elucidate the molecular mechanism of action (MoA) of CBD towards identifying direct target(s).

Despite the legal hurdled that have accompanied CBD research in the past several previous studies have broadly examined the large-scale effects of CBD in cultured cells. For instance, transcriptomics has been used to determine that CBD alters both metal ion and cholesterol homeostasis in microglial cells. Another recent study reported that CBD-induced apoptosis in two human neuroblastoma cell lines could be inhibited by co-treatment with serotonin and vanilloid receptor antagonists, and showed a metabolic shift towards glycolysis, highlighting the potential pleiotropic effects from CBD exposure. Although these studies have advanced the understanding of CBD, they have not produced a consistent model of the MoA of CBD that explains the apparent pleiotropic cellular effects and clinical therapeutic effects that have been shown and are currently under investigation. Indeed, as the MoA is better understood, it opens the possibility for additional therapeutic uses of CBD and its analogs. For example, a clearer understanding of the CBD's MoA may allow it to be directed to the treatment of various cholesterol-related disease conditions, such as cardiovascular diseases (CVD).

CVD are the major cause of death and disability in the United States and other industrialized countries despite recent declines in CVD mortality rates. They account for more deaths annually than any other disease, including all forms of cancer combined. In the USA more than 1 million heart attacks occur each year and more than half a million people still die as a result. This enormous toll has focused attention on the possible prevention of CVD by various means, especially through lowering of plasma cholesterol levels. It is now well established that elevated total cholesterol and in particular low-density lipoprotein (LDL) cholesterol, in plasma plays an important role in the development of atherosclerosis. Clinical trials have demonstrated clearly that decreasing cholesterol concentrations in plasma can contribute to primary and secondary prevention of coronary events and mortality. Some studies have estimated a 2% reduction in risk of a coronary artery event by a 1% reduction of total serum cholesterol.

To combat CVD, and other cholesterol-related conditions, certain pharmaceutical compounds have been developed to modulate cholesterol levels in a patient. For example, compositions that inhibit the cholesterol biosynthesis by inhibiting enzyme 3-hydroxy-3-methylglutaryl coenzyme A reductase (HMG-CoA reductase), an enzyme involved in the cholesterol biosynthesis, can lower blood serum cholesterol by slowing down the production of cholesterol. It is believed that inhibition of HIMG-CoA reductase results in a reduction in hepatic cholesterol synthesis and intracellular cholesterol stores, a compensatory increase in low-density lipoprotein (LDL) receptors, and a subsequent enhanced removal of LDL-cholesterol from plasma. Potent inhibitors of HMG-CoA reductase include for example the compounds referred to as statins, which family comprises atorvastatin, lovastatin, pravastatin and fluvastatin. However, statins are not without limitations. For example, statins can cause type 2 diabetes or higher blood sugar, confusion and memory loss, as well as liver, muscle and/or kidney damage. Accordingly, there exists a need for novel treatment methods of CVD, and other cholesterol-related conditions.

As demonstrated below, the present inventors have identified the key biochemical effectors of CBD in human cells using a broad search strategy employing transcriptomics, proteomics, metabolomics, and live cell microscopy. The inventors discovered thousands of CBD dependent molecular changes within cells that highlighted a central theme around cholesterol homeostasis. In addition, the present inventors discovered that CBD incorporates into the plasma membrane and not only triggers transport and storage of cholesterol to lipid droplets, but also may cause blockage of cholesterol biosynthesis in the final step of the biosynthetic pathway. The inventors additionally found that CBD can affect lipid order by altering the orientation of cholesterol within synthetic and cell derived membrane models. The inventor's study demonstrates that the observed downstream effects of CBD on cholesterol homeostasis are triggered by the lipid order effects of CBD at the plasma membrane. This model challenges the previous models for the CBD MoA, as it proposes that cholesterol may be the primary pharmacological effector rather than any given protein receptor. Based on this new and unexpected understanding, the present inventors may more effectively employ CBD, and its analogs, in the treatment of cholesterol-related diseases, such as CVD among others.

### SUMMARY OF THE INVENTION

The present invention demonstrates the successful use of a multi-omic method to produce a data driven model for the MoA of CBD. The inventor's multidisciplinary approach revealed mechanistic components in the CBD MoA to be concentrated in cholesterol homeostasis in cells. Specifically, this disclosure details the exact molecular features of cholesterol homeostasis that are altered, including intracellular transport, storage, and biosynthesis.

The inventors further provide evidence that the origin of these effects is CBD's direct effect on cholesterol and lipid order. Within cells, lipid ordered domains (also referred to as lipid rafts) are 10-120 nm wide transient domains in membrane structures where tight packing of cholesterol, sphingolipids and GPI-anchored proteins allow for activation of signaling cascades via the increased proximity of specific proteins. These ordered domains have been implicated in generating receptor signaling, assembly of endocytic machinery and endocytosis, and regulation of ion channels. In one embodiment, the present inventor demonstrate that CBD incorporates into the plasma membrane and affects cholesterol orientation and lateral diffusion in membrane model systems and cell derived endoplasmic reticulum (ER) membranes. This effect initiates the vast amount of downstream effects of CBD, which include cholesterol dependent apoptosis, increased cholesterol storage and trafficking, and multiple effects on the cholesterol biosynthesis pathway. Altogether, the inventor's multi-omic study concludes that cholesterol is a major effector of CBD, which may explain the diverse clinical applications for CBD proposed in previous studies, where membrane cholesterol plays a widespread underappreciated role in disease progression.

One aspect of the invention includes novel systems, methods, and compositions for the modulation of cholesterol in a patient. In certain preferred embodiment, the modulation of cholesterol in a patient may be accomplished through the administration of a therapeutically effective amount of a cannabinoid compound and/or a cannabinoid analog to a patient. In this preferred embodiment, the modulation of cholesterol in a patient may be accomplished through the administration of a therapeutically effective amount of a cannabidiol (CBD) compound and/or a CBD analog to a patient. In certain preferred embodiments, CBD analogs may include, but not be limited to: HU-308, o-1821, o-1602, and/or abnormal CBD. Additional embodiments may further include cannabinol, natural and synthetic analogs of THC (Marinol, nabilone, Ajulemic Acid, DHM-CBD, deoxy0CBD, 11-hydroixy-CBD, CBD-11-oic acid; CP 55,940, HU-210, HU-211, HU-308, HU331, and/or 11-hydroxy-Δ9-THC).

Another aspect of the invention may include the use of one or more cannabinoid and/or cannabinoid analogs alone or in combination with a cholesterol treatment. In one preferred embodiment, the invention may include the use of CBD and/or a CBD analog alone, or in combination with a cholesterol regulation medication, such as a statin.

Another aspect of the invention includes novel systems, methods, and compositions for the modulation of cholesterol in a patient through the administration of a therapeutically effective amount of a cannabinoid compound and/or a cannabinoid analog to a patient in combination with a separate cholesterol treatment. In this preferred embodiment, the modulation of cholesterol in a patient may be accomplished through the administration of a therapeutically effective amount of CBD and/or a CBD analog to a patient in combination with, for example, a HMG-CoA reductase inhibitor, which may also be referred to as a statin.

Another aspect of the current invention may include the use of one or more cannabinoid and/or cannabinoid analogs alone, or in combination with a cholesterol treatment to lower the level of cholesterol in blood serum in a patient. In one preferred embodiment, aspect of the current invention may include the use CBD and/or a CBD analog alone, or in combination with a cholesterol regulation medication to lower the level of cholesterol in blood serum in a patient.

Another aspect of the current invention may include novel methods, systems and compositions to modulate cholesterol to treat one or more cholesterol-related conditions in a patient. Examples of such conditions may include, but not be limited to: cardiovascular disease, type I diabetes, type II diabetes, obesity, hypertension, stroke, peripheral arterial disease (PAD), dyslipidemia, hyperlipidemia, hyperlipoproteinemia, hypolipidemia, hypolipoproteinemia and other cholesterol-related conditions that may present in a patient.

Another aspect of the invention includes modulating cholesterol sensing, production and trafficking within cell, tissue and/or patient. In certain embodiments, administration of one or more cannabinoid and/or cannabinoid analogs may induce structural changes in cellular endoplasmic reticulum. In one preferred embodiment, the invention includes modulating cholesterol sensing, production and trafficking within cell, tissue and/or patient through the administration of a therapeutically effective amount of CBD and/or CBD analog. In certain embodiments, administration of a therapeutically effective amount of CBD and/or CBD analogs may induce structural changes in cellular endoplasmic reticulum.

Another aspect of the invention includes modulating cholesterol content of cell membranes, plaque within and outside of cells in tissues of patients. Such plaques include disordered and ordered water insoluble molecular aggregates that are comprised of cholesterol and other lipids in combination with proteins, RNA and DNA, or combinations of two or more of these molecular classes with cholesterol. In certain embodiment, administration of one or more cannabinoid and/or cannabinoid analogs may induce dissolution of plaques, or alter the mobility or solubility of cholesterol within membranes. In additional embodiment, administration of a therapeutically effective amount of CBD and/or CBD analogs may induce dissolution of plaques, or alter the mobility or solubility of cholesterol within membranes.

Additional aspects of the invention may also include:
1. A method of modulating the level of cholesterol in a cell comprising the step of:
   introducing an effective amount of a cannabidiol (CBD) or a CBD analog to a cell, wherein said effective amount induces one or more of the following:
      increasing the lipid order of cholesterol containing cell membranes in said cell;
      increasing cellular cholesterol storage in said cell;
      altering the orientation of cholesterol present in lipid membranes in said cell;
      increasing cellular endoplasmic reticulum cholesterol transport in said cell;
      increasing cellular endoplasmic reticulum cholesterol storage in said subject;
      activating SREBP-SCAP processing in said cell;
      increasing production of HMG-CR in said cell;
      inhibiting DHCR24 in said subject sufficient to disrupt cholesterol biosynthesis in said cell; and
      inhibiting DHCR7 sufficient to disrupt cholesterol biosynthesis in said cell.
   and wherein said effective amount does not induce apoptosis in said cell.
2. The method of embodiment 1 wherein said cell is a human cell.
3. The method of embodiment 2 wherein said step of introducing an effective amount of a CBD or a CBD analog to a cell comprises the step of introducing an effective amount of a CBD or a CBD analog to a cell in vivo, in vitro or ex vivo.
4. The method of embodiment 1 wherein said CBD or a CBD analog is isolated from a Cannabis plant.
5. The method of embodiment 1 wherein said CBD or a CBD analog is synthetically manufactured.
6. The method of embodiment 1 and further comprising the step of administrating a therapeutically effective amount of a cholesterol regulation medication to said subject.
7. The method of embodiment 6 wherein said cholesterol regulation medication is a statin.
8. The method of embodiment 1 wherein said CBD analog comprises a CBD analog selected from the group consisting of: HU-308, o-1821, o-1602, abnormal CBD, ajulemic Acid, DHM-CBD, deoxy0CBD, 11-hydroxy-CBD, CBD-11-oic acid; CP 55,940, HU-210, HU-211, HU331, and/or 11-hydroxy-Δ9-THC.
9. A method of modulating the level of cholesterol in a subject comprising the step of:
   administering a therapeutically effective amount of cannabidiol (CBD) or a CBD analog to a subject, wherein said subject is suffering from, or predisposed to developing a cholesterol-related condition and wherein said therapeutically effective amount induces one or more of the following:
      increasing the lipid order of cholesterol containing cell membranes in said subject;
      increasing cellular cholesterol storage in said subject;
      altering the orientation of cholesterol present in lipid membranes in said subject;
      increasing cellular endoplasmic reticulum cholesterol transport in said subject;
      increasing cellular endoplasmic reticulum cholesterol storage in said subject;
      activating SREBP-SCAP processing in said subject;
      increasing production of HMG-CR in said cell;
      inhibiting DHCR24 in said subject sufficient to disrupt cholesterol biosynthesis in said subject; and
      inhibiting DHCR7 sufficient to disrupt cholesterol biosynthesis in said subject.
   and wherein said therapeutically effective amount does not induce cell apoptosis.
10. The method of embodiment 9 wherein said cell is a human cell.
11. The method of embodiment 10 wherein said step of introducing an effective amount of a CBD or a CBD analog to a cell comprises the step of introducing an effective amount of a CBD or a CBD analog to a cell in vivo, in vitro or ex vivo.
12. The method of embodiment 9 wherein said CBD or a CBD analog is isolated from a Cannabis plant.
13. The method of embodiment 9 wherein said CBD or a CBD analog is synthetically manufactured.
14. The method of embodiment 9 and further comprising the step of administrating a therapeutically effective amount of a cholesterol regulation medication to said subject.
15. The method of embodiment 14 wherein said cholesterol regulation medication is a statin.
16. The method of embodiment 9 wherein said CBD analog comprises a CBD analog selected from the group consisting of: HU-308, o-1821, o-1602, abnormal CBD, ajulemic Acid, DHM-CBD, deoxy0CBD, 11-hydroxy-CBD, CBD-11-oic acid; CP 55,940, HU-210, HU-211, HU331, and/or 11-hydroxy-Δ9-THC.
17. A method of treating a subject having a disease condition comprising the step of administering a therapeutically effective amount of isolated cannabidiol (CBD) or a CBD analog, wherein said therapeutically effective amount treats one or more disease indications:
   deficient lipid order of cholesterol containing cell membranes in said subject;
   deficient cellular cholesterol storage in said subject;
   altering the orientation of cholesterol present in lipid membranes in said subject;
   deficient cellular endoplasmic reticulum cholesterol transport in said subject;
   deficient cellular endoplasmic reticulum cholesterol storage in said subject;
   low SREBP-SCAP processing activity in said subject;
   deficient production of HMG-CR in said subject;
   excess activity or expression DHCR24 resulting in excess cholesterol biosynthesis in said subject; and
   excess activity or expression of DHCR7 resulting in excess cholesterol biosynthesis in said subject.
   and wherein said therapeutically effective amount does not increase cellular apoptosis in said subject.
18. The method of embodiment 17 wherein said cannabinoid comprises CBD and/or a CBD analog.
19. The method of embodiment 18 wherein said CBD analog is selected from the group consisting of: wherein said CBD analog comprises a CBD analog selected from the group consisting of: HU-308, o-1821, o-1602, abnormal CBD, ajulemic Acid, DHM-CBD, deoxy0CBD, 11-hydroxy-CBD, CBD-11-oic acid; CP 55,940, HU-210, HU-211, HU331, and/or 11-hydroxy-Δ9-THC.
20. The method of embodiment 19 wherein said disease condition comprises a cholesterol-related condition.
21. The method of embodiment 20 wherein said subject is a human.
22. The method of embodiment 21 wherein said therapeutically effective amount of CBD or a CBD analog further comprises the indication of reducing the level of cholesterol in said subject.
23. The method of embodiment 21 wherein said therapeutically effective amount of CBD or a CBD analog further comprises the indication of reducing the level of cholesterol in said subject at a level comparable to administering a therapeutically effective amount of a statin.
24. The method of embodiment 18 wherein said step of introducing an effective amount of a CBD or a CBD analog to a cell comprises the step of introducing an effective amount of a CBD or a CBD analog to a cell in vivo, in vitro or ex vivo.
25. The method of embodiment 17 wherein said CBD or a CBD analog is isolated from a Cannabis plant.
26. The method of embodiment 17 wherein said CBD or a CBD analog is synthetically manufactured.
27. The method of embodiment 17 and further comprising the step of co-administrating a therapeutically effective amount of a cholesterol regulation medication to said subject.
28. The method of embodiment 27 wherein said cholesterol regulation medication is a statin.
29. A method of treating a subject having a lipid-order disease condition comprising the step of administering a therapeutically effective amount of cannabidiol (CBD) or a CBD analog, wherein said therapeutically effective amount increases the lipid order of cholesterol containing cell membranes in said subject.
30. The method of embodiment 29 wherein said lipid-order disease comprises Alzheimer's disease.
31. The method of embodiment 30 wherein said subject is a mammal.
32. The method of embodiment 32 wherein said mammal is a human.
33. The method of embodiment 20 wherein said therapeutically effective amount does not increase levels of cellular apoptosis.
34. The method of embodiment 29 wherein said CBD analog is selected from the group consisting of: wherein said CBD analog comprises a CBD analog selected from the group consisting of: HU-308, o-1821, o-1602, abnormal CBD, ajulemic Acid, DHM-CBD, deoxy0CBD, 11-hydroxy-CBD, CBD-11-oic acid; CP 55,940, HU-210, HU-211, HU331, and/or 11-hydroxy-Δ9-THC
35. The method of embodiment 29 wherein said CBD or a CBD analog is isolated from a Cannabis plant.
36. The method of embodiment 29 wherein said CBD or a CBD analog is synthetically manufactured.
37. The method of embodiment 29 and further comprising the step of administrating a therapeutically effective amount of a lipid-ordering compound.
38. The method of embodiment 30 and further comprising the step of co-administrating a therapeutically effective amount of a Alzheimer's disease controlling compound selected from the group consisting of: a cholinesterase inhibitor; a NMDA inhibitor, donepezil, memantine, donepezil, galantamine, and rivastigmine.
39. A method of enhancing a T-Cell immune response in a subject comprising the step of administering a therapeutically effective amount of isolated cannabidiol (CBD) or a CBD analog, wherein said therapeutically effective amount causes a proliferation of T-cells in said subject.
40. The method of embodiment 39 wherein said subject is a mammal.
41. The method of embodiment 40 wherein said mammal is a human.
42. The method of embodiment 39 wherein said wherein said therapeutically effective amount does not increase levels of cellular apoptosis.
43. The method of embodiment 39 wherein said CBD analog is selected from the group consisting of: wherein said CBD analog comprises a CBD analog selected from the group consisting of: HU-308, o-1821, o-1602, abnormal CBD, ajulemic Acid, DHM-CBD, deoxy0CBD, 11-hydroxy-CBD, CBD-11-oic acid; CP 55,940, HU-210, HU-211, HU331, and/or 11-hydroxy-Δ9-THC
44. The method of embodiment 39 wherein said CBD or a CBD analog is isolated from a Cannabis plant.
45. The method of embodiment 29 wherein said CBD or a CBD analog is synthetically manufactured.
46. The method of embodiment 29 and further comprising the step of administrating a therapeutically effective amount of a lipid-ordering compound.
47. A method of disrupting cellular cholesterol homeostasis in a subject comprising the steps of administering a therapeutically effective amount of cannabidiol (CBD) or a CBD analog to a subject, wherein said subject is suffering from, or predisposed to developing a disease condition that may be treated by the disruption of cellular cholesterol homeostasis.
48. The method of embodiment 47 wherein said disruption of cellular cholesterol homeostasis comprises a reduction in cholesterol in a subject.
49. A method of increasing the lipid order of a cell membrane of a subject comprising the steps of administering a therapeutically effective amount of cannabidiol (CBD) or a CBD analog to a subject, wherein said subject is suffering from, or predisposed to developing a disease condition that may be treated by said increase in the lipid order of a cell membrane.
50. A method of increasing the lipid order of a cell membrane comprising the step of introducing to said cell an effective amount of cannabidiol (CBD) or a CBD analog.
51. A method of increasing cholesterol storage in a cell of a subject comprising the steps of administering a therapeutically effective amount of cannabidiol (CBD) or a CBD analog to a subject, wherein said subject is suffering from, or predisposed to developing a disease condition that may be treated by said increasing cholesterol storage in a cell of a subject.
52. A method of increasing cholesterol storage in a cell comprising the step of introducing to said cell an effective amount of cannabidiol (CBD) or a CBD analog.
53. A method of altering the orientation of cholesterol present in a lipid membrane of a subject comprising the steps of administering a therapeutically effective amount of cannabidiol (CBD) or a CBD analog to a subject, wherein said subject is suffering from, or predisposed to developing a disease condition that may be treated by said altering the orientation of cholesterol present in a lipid membrane of a subject.
54. A method of altering the orientation of cholesterol present in a lipid membrane in a cell comprising the step of introducing to said cell an effective amount of cannabidiol (CBD) or a CBD analog.
55. A method of increasing cholesterol precursors in a cell of a subject comprising the steps of administering a therapeutically effective amount of cannabidiol (CBD) or a CBD analog to a subject, wherein said subject is suffering from, or predisposed to developing a disease condition that may be treated by said increasing cholesterol precursors in a cell of a subject.
56. A method of increasing cholesterol precursors in a cell comprising the step of introducing to said cell an effective amount of cannabidiol (CBD) or a CBD analog.
57. A method of increasing cholesterol transport and/or storage to a cell's endoplasmic reticulum of a subject comprising the steps of administering a therapeutically effective amount of cannabidiol (CBD) or a CBD analog to a subject, wherein said subject is suffering from, or predisposed to developing a disease condition that may be treated by increasing cholesterol transport and storage to a cell's endoplasmic reticulum.
58. A method of increasing cholesterol transport and/or storage to a cell's endoplasmic reticulum comprising the step of introducing to said cell an effective amount of cannabidiol (CBD) or a CBD analog.
59. A method of inhibiting the cholesterol biosynthesis in a subject comprising the steps of administering a therapeutically effective amount of cannabidiol (CBD) or a CBD analog to a subject, wherein said subject is suffering from, or predisposed to developing a disease condition that may be treated by said inhibiting the cholesterol biosynthesis in a subject.
60. A method of inhibiting the cholesterol biosynthesis comprising the step of introducing to said cell an effective amount of cannabidiol (CBD) or a CBD analog.
61. A method of inhibiting DHCR24 comprising the steps of administering a therapeutically effective amount of cannabidiol (CBD) or a CBD analog to a subject, wherein said subject is suffering from, or predisposed to developing a disease condition that may be treated by inhibiting DHCR24.
62. A method of inhibiting DHCR24 comprising the step of introducing to said cell an effective amount of cannabidiol (CBD) or a CBD analog.
63. A method of inhibiting DHCR7 comprising the steps of administering a therapeutically effective amount of cannabidiol (CBD) or a CBD analog to a subject, wherein said subject is suffering from, or predisposed to developing a disease condition that may be treated by inhibiting DHCR24.
64. A method of inhibiting DHCR7 comprising the step of introducing to said cell an effective amount of cannabidiol (CBD) or a CBD analog.
65. A method of activating SREBP-SCAP processing comprising the steps of administering a therapeutically effective amount of cannabidiol (CBD) or a CBD analog to a subject, wherein said subject is suffering from, or predisposed to developing a disease condition that may be treated by activating SREBP-SCAP processing.
66. A method of activating SREBP-SCAP processing comprising the step of introducing to said cell an effective amount of cannabidiol (CBD) or a CBD analog.
67. A method of increasing production of HMG-CR comprising the steps of administering a therapeutically effective amount of cannabidiol (CBD) or a CBD analog to a subject, wherein said subject is suffering from, or predisposed to developing a disease condition that may be treated by increasing production of HMG-CR.
68 A method of increasing production of HMG-CR comprising the step of introducing to said cell an effective amount of cannabidiol (CBD) or a CBD analog.
69. The method of embodiments 47-68 wherein said subject is a mammal.
70. The method of embodiments 69 wherein said mammal is a human.
71. The method of embodiments 47, 49, 51, 53, 55, 57, 59, 61, 63, 65, and 67 wherein said therapeutically effective amount does not increase levels of cellular apoptosis.
72. The method of embodiments 50, 52, 54, 56, 58, 60, 62, 64, 66, 68 wherein said effective amount does not increase levels of cellular apoptosis.
73. The method of embodiments 47-68 wherein said CBD analog comprises a CBD analog selected from the group consisting of: HU-308, o-1821, o-1602, abnormal CBD, ajulemic Acid, DHM-CBD, deoxy0CBD, 11-hydroxy-CBD, CBD-11-oic acid; CP 55,940, HU-210, HU-211, HU331, and/or 11-hydroxy-Δ9-THC.
74. The method of embodiments 47-68 wherein said CBD or a CBD analog is isolated from a Cannabis plant.
75. The method of embodiments 47-68 wherein said CBD or a CBD analog is synthetically manufactured.
76. A method of modulating the level of cholesterol in a subject comprising the step of:
   administering a therapeutically effective amount of a cannabinoid to a subject, wherein said subject is suffering from, or predisposed to developing a cholesterol-related condition and wherein said therapeutically effective amount induces one or more of the following:
      increasing the lipid order of cholesterol containing cell membranes in said subject;
      increasing cellular cholesterol storage in said subject;
      altering the orientation of cholesterol present in lipid membranes in said subject;
      increasing cellular endoplasmic reticulum cholesterol transport in said subject;
      increasing cellular endoplasmic reticulum cholesterol storage in said subject;
      activating SREBP-SCAP processing in said cell;
      increasing production of HMG-CR in said cell;
      inhibiting DHCR24 in said subject sufficient to disrupt cholesterol biosynthesis in said subject; and
      inhibiting DHCR7 sufficient to disrupt cholesterol biosynthesis in said subject.
77. A method of modulating the level of cholesterol in a cell comprising the step of:
   introducing an effective amount of a cannabinoid to a cell, wherein said effective amount induces one or more of the following:
      increasing the lipid order of cholesterol containing cell membranes in said cell;
      increasing cellular cholesterol storage in said cell;
      altering the orientation of cholesterol present in lipid membranes in said cell;
      increasing cellular endoplasmic reticulum cholesterol transport in said cell;
      increasing cellular endoplasmic reticulum cholesterol storage in said subject;
      activating SREBP-SCAP processing in said cell;
      increasing production of HMG-CR in said cell;
      inhibiting DHCR24 in said subject sufficient to disrupt cholesterol biosynthesis in said cell; and
      inhibiting DHCR7 sufficient to disrupt cholesterol biosynthesis in said cell.
78. The method of embodiments 1-78 above and further comprising co-administering to a subject one or more additional cannabinoids and/or one or more additional terpenoids.
79. The method of embodiments 1-78 wherein said CBD or CBD analog is glycosylated.
80. The method of embodiments 1-78 wherein said CBD or CBD analog is an acetylated glycoside.
81. The method of embodiments 79-89 wherein said glycosylated and/or acetylated glycoside CBD or CBD analog is administered as a pro-drug.
82. A method of increasing the lipid order of a cell membrane in vivo comprising the step of introducing to said cell an effective amount of cannabidiol (CBD) or a CBD analog, wherein said increasing the lipid order of a cell membrane occurs in the absence of sphingolipids and/or sphingomyelin.
83. A method of increasing the lipid order of a cell membrane in vivo comprising the step of introducing to said cell an effective amount of cannabidiol (CBD) or a CBD analog wherein said increasing the lipid order of a cell membrane results in the formation of increase and/or more stable lipid rafts in said cell membrane.

Additional aims of the inventive technology will be evident from the detailed description and figures presented below.

### BRIEF DESCRIPTION OF DRAWINGS

The novel aspects, features, and advantages of the present disclosure will be better understood from the following detailed descriptions taken in conjunction with the accompanying figures, all of which are given by way of illustration only, and are not limiting the presently disclosed embodiments, in which:

FIG. 1A-D. Achematic of multi-omic experimental design to analyze the CBD response in SK-N-BE(2) cells. CBD (A), was assessed for its long term cytotoxicity (B). (C) Dose and time point selection for multi-omics experiments was determined using a broad multiplexed phenotypic screen with a panel of FRET biosensor expressing cell lines. (D) Technical and output metrics for transcriptomics, metabolomics, and proteomics experiments.

FIG. 2A-D. Analysis and classification of biomolecules modulated in the CBD cellular response using transcriptomics and subcellular proteomics. Intact SKNBE2 cells were fractionated into chemically distinct fractions using centrifugation and modulation of buffer pH (A). (B) The resulting fractions were assessed for the total number of detectable protein identities, as well as the total number of proteins affected by CBD in each fraction as a function of time (C). Filtered CBD responsive proteins are displayed in D in combination with CBD responsive mRNA transcripts identified in transcriptomics. Each protein/mRNA transcript ID was classified into representative gene ontologies to determine broad cellular processes affected by the CBD response.

FIG. 3A-C. CBD's affects on Cholesterol Biosynthesis. (A) A diagram of the cholesterol biosynthesis pathway. The canonical pathway is indicated with bold arrows. Dotted arrows indicate multiple steps. (B) Newly Synthesized lipids with isotopic incorporation of C¹³ from C¹³ labelled glucose in SK-N-BE(2) cells was analyzed in the presence and absence of 24 hour CBD stimulation (20 μM) for cholesterol and precursors in the biosynthesis of cholesterol. (C) Western blot analysis of SREBP proteolytic processing in whole cell lysates after 24 hr stimulation of CBD, Atorvastatin, and U18666A as indicated.

FIG. 4A-G. CBD Activates Cholesterol Esterification and Storage. Total lipid abundance extracted from SK-N-BE (2) cells stimulated with vehicle or CBD (20 μM) was analyzed for cholesteryl esters (A), free fatty acids (B), free head groups (C), and phospho-lipids (D). (E) Live cell confocal microscopy of SK-N-BE(2) cells exposed to CBD (20 μM) stained with fluorescent cholesterol and nile red. Scale bar 5 Quantification of lipid droplet count per cell and lipid droplet size from confocal experiments is displayed in (F) and (G), respectively.

FIG. 5A-F. The Response to CBD Depends On The Cholesterol Biosynthesis/Transport/Storage Network. Cells exposed to vehicle or Atorvastatin (10 μM) were assessed for dose dependent induction of apoptosis using live cell microcopy in SKNBE2 cells (A) and HaCaT cells (B). Similar experiments were conducted with 25-OH cholesterol substituted for Atorvastatin (C-D). (E) SKNBE2 and 293T cells were assessed for the ability of the small molecule inhibitors U18666A (10 μM) and VULM 1457 (5 μM) to affect CBD induced apoptosis in the presence or absence of 25-OH cholesterol at 24 hours. (F) Live cell confocal microscopy using fluorescent NBD-Cholesterol (NBD-Chol.) and a lysosomal dye (Lyso-T) were used to visualize cholesterol subcellular distribution upon systematic exposure of cells to CBD (20 μM) and/or U18666A (10 μM). Scale bar 3 μm.

FIG. 6A-I. In vitro characterization of CBD's ability to affect cholesterol orientation and lateral diffusion in membranes. (A) Ethanol extracts of subcellular fractions of SK-N-BE(2) cells exposed to 0, 20 and 40 μM CBD were analyzed for CBD using LC-MS. (B) Synthetic small unilamellular vesicles (SUVs) were used as a source of cholesterol in a fluorogenic cholesterol oxidase reaction to determine the affect of CBD on initial reaction rate. Identical experiments were performed with cholesterol complexed to methyl beta cyclodextrin (MBCD) (C) and free 25-OH cholesterol (25-OH Chol.)(D). Vesicles of ER membrane were isolated from SK-N-BE(2) cells, stained with ER-tracker dye to be visualized using confocal microscopy (E). ER derived vesicles were used as a source of cholesterol for similar experiments as displayed in B-D. (F) Synthetic membrane monolayers containing NBD-cholesterol were adsorbed to borosilicate glass and used in fluorescent recovery after photobleaching (FRAP) experiments following exposure to either CBD (60 μM) and/or DHA (20 μM). Scale bar is 2.5 μm. Quantitative analysis of F is displayed in G and H.

FIG. 7A-D. DHA induces cholesterol dependent apoptosis that can be inhibited by CBD treatment. (A) The percent of Apoptosis was assessed in HEK293T cells as a function of DHA dose and time. (B) A similar experiment was conducted with SK-N-BE(2) cells. (C) Similar experiments were conducted with HEK293T cells (C) or SK-N-BE(2) cells (D) exposed to high dose DHA (75 μM) and either MBCD (300 μM) or low dose CBD (6.25 μM).

FIG. 8. A Data Driven Model of CBD MoA in Cells. 1) CBD embeds in the plasma membrane. 2) Ordered domains form containing CBD and cholesterol. 3) Ordered domains are endocytosed. 4) Cholesterol is trafficking through the lysosome to the ER. 5) Cholesterol in the ER inhibits DHCR7/24 completion of cholesterol biosynthesis and activates cholesterol esterification/storage.

FIG. 9A-C. Biosensor Profiling of CBD. (A, B, C) Genetically encoded FRET biosensors for diverse biochemical pathways were delivered to both SK-N-BE(2) and HaCaT cells. Biosensor activity for each biosensor expressing cell line was analyzed as a function of time and dose of CBD exposure

FIG. 10. Biosensor Profiling of EC50 Effects of CBD. (B) The EC50 of CBD's effect on biosensor activity was analyzed for each biosensor at each time point, where EC50's resulting from data fitting with R2>0.75 are displayed.

FIG. 11A-C. (A) Plots of 13C labelled (left) and total+labeled cholesterol (right) abundances measured in methanol extracts from vehicle/CBD treated cells. (B) A similar analysis for cholesterol precursors. (C) Fluorometric measurement of total cellular cholesterol from methanol extracts of SK-N-BE(2) cells using the Amplex Red Cholesterol Assay Kit.

FIG. 12A-L. Kinetic analysis of CBD induced apoptosis in multiple cells types exposed to chemical perturbants of cholesterol flux. SKNBE2 cells exposed to vehicle or the HMG-CR inhibitor Atorvastatin were assessed for time dependent induction of apoptosis using live cell microscopy of a fluorogenic caspase 3/7 dye (A,B). Identical analysis was performed on HaCaT Cells (C,D). Similar experiments were conducted for SKNBE2 and HaCaT cells, with 25-OH cholesterol substituted for Atorvastatin (E-H). SKNBE2 cells and 293T cells were assessed for the ability of the small molecule inhibitors U18666A and VULM 1457 to affect CBD induced apoptosis in the presence or absence of 25-OH cholesterol (I-L).

FIG. 13. Synthetic small unilamellular vesicles (SUVs) free of cholesterol and composed of 100% Phosphatidyl Choline were analyzed with a fluorogenic cholesterol oxidase reaction to determine the affect of CBD on initial reaction rate in the presence and absence of cholesterol oxidase enzyme.

FIG. 14A-H. Flow cytometric analysis of rhodamine-glibenclamide staining in live cells. Enhanced fluorescence is observed for the cholesterol reducing drugs Atorvastatin(B) and Ezetimibe(C), as well as CBD (A) and exemplary analogs of CBD: abnormal CBD(D), Cannabidiolic Acid(E), HU-308 (F), o-1821 (G), and o-1602 (H).

FIG. 15. Toxicity profiling. Demonstrates percent death after 24 hrs of exposure of CBD or CBD analogs in cells with and without cholesterol challenge;

FIG. 16. Rhodamine-glibenclamide staining in live cells is visualized by confocal microscopy in live cells. ER structure is perturbed after 2 hours of exposure to CBD and o-1602, but not for o-1821 exposure.

FIG. 17A-F. Cholesterol oxidase reaction rate as measured through Amplex Red/Horse Radish Peroxidase mediated detection of hydrogen peroxide produced by cholesterol oxidase. ER derived membrane vesicular bodies (ERVBs) are extracted from living neuroblastoma cells and exposed to CBD, CBD analogs, terpenes, or the cholesterol sequestering chemical methyl beta cyclodextrin (MBCD).

### DETAILED DESCRIPTION OF INVENTION

The following detailed description is provided to aid those skilled in the art in practicing the various embodiments of the present disclosure, including all the methods, uses, compositions, etc., described herein. Even so, the following detailed description should not be construed to unduly limit the present disclosure, as modifications and variations in the embodiments herein discussed may be made by those of ordinary skill in the art without departing from the spirit or scope of the present discoveries.

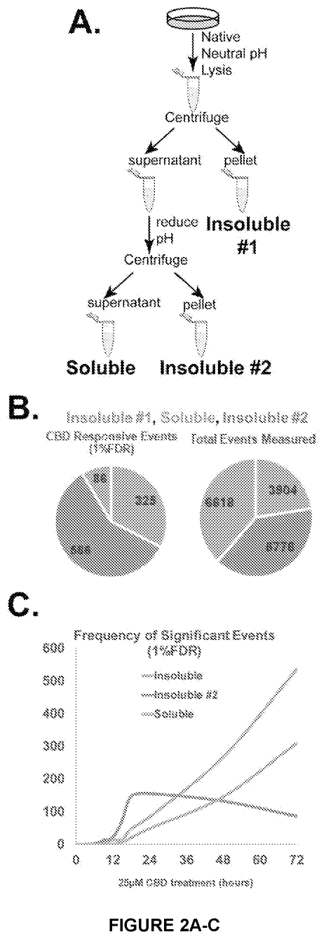

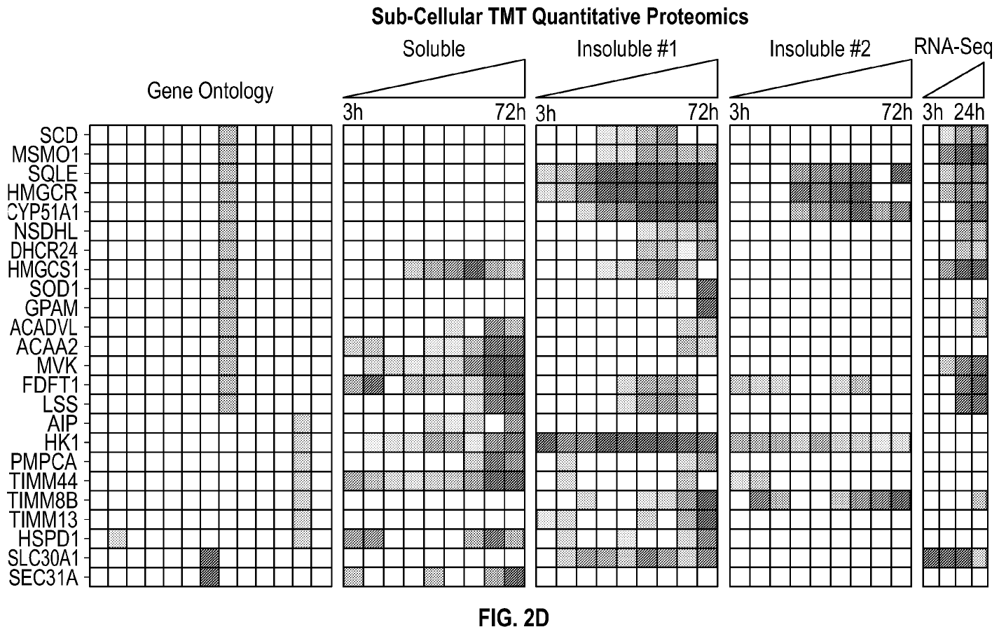

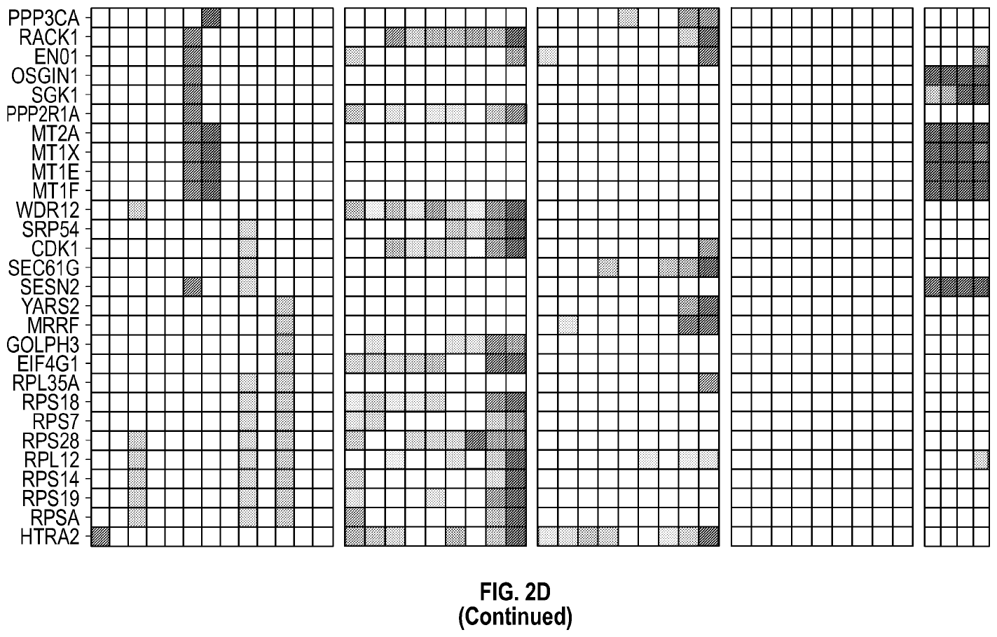

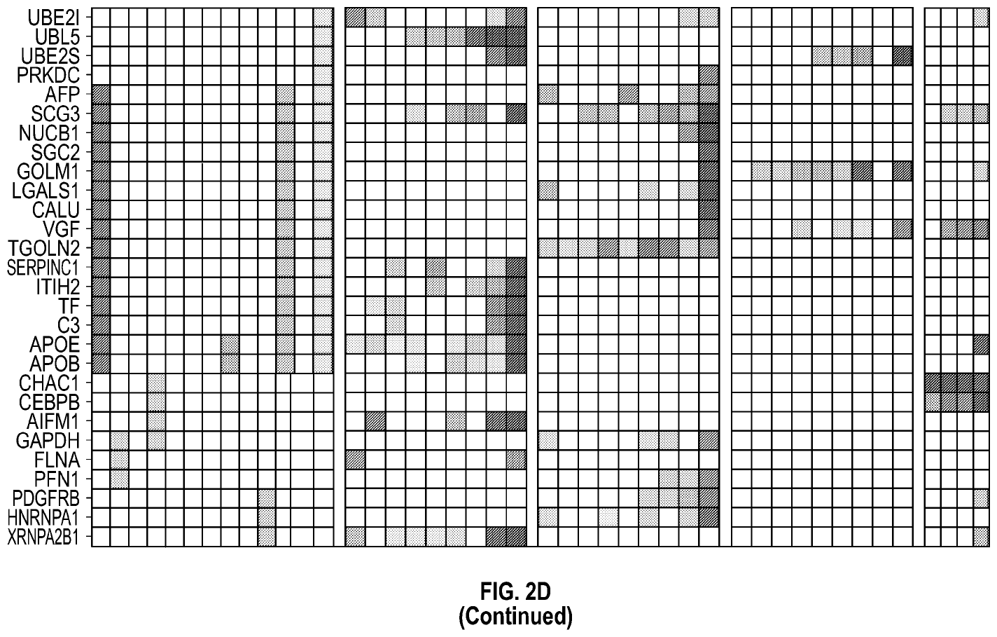

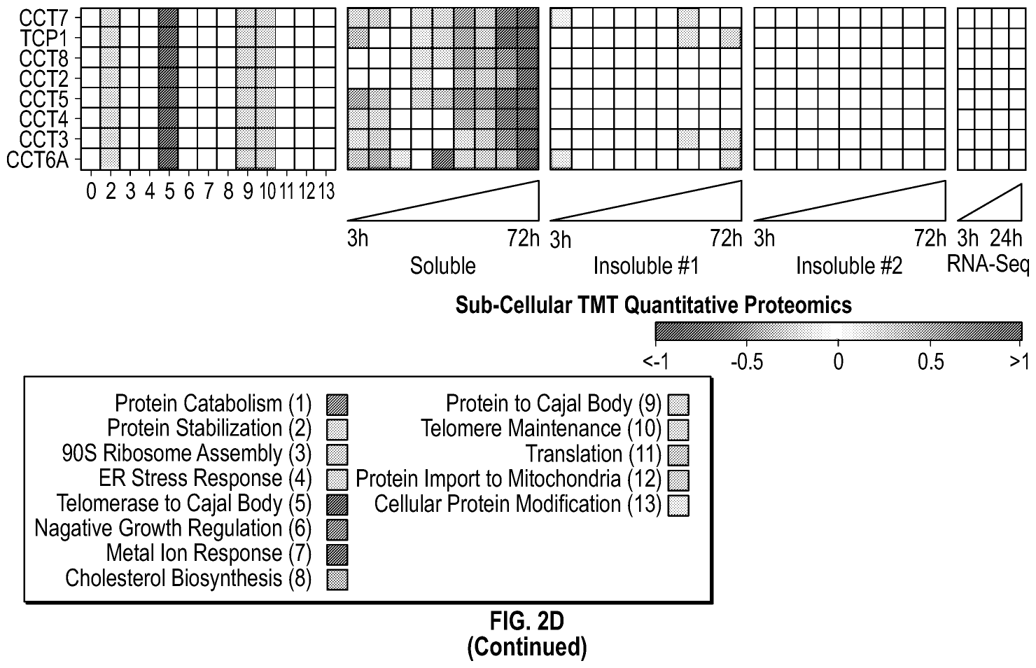

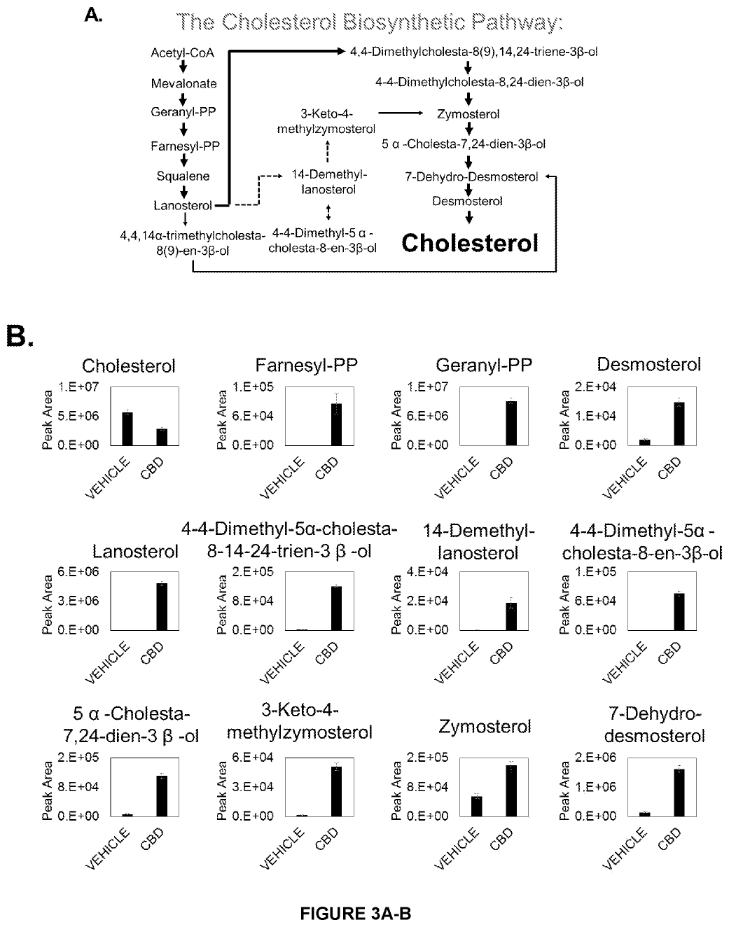

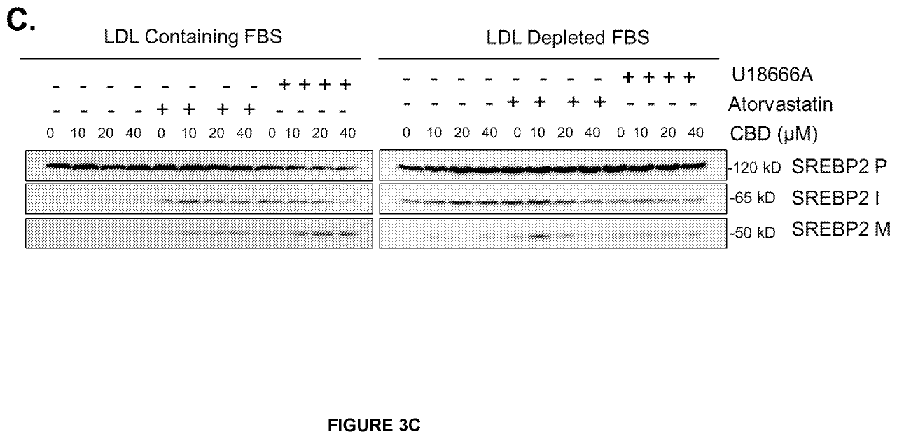

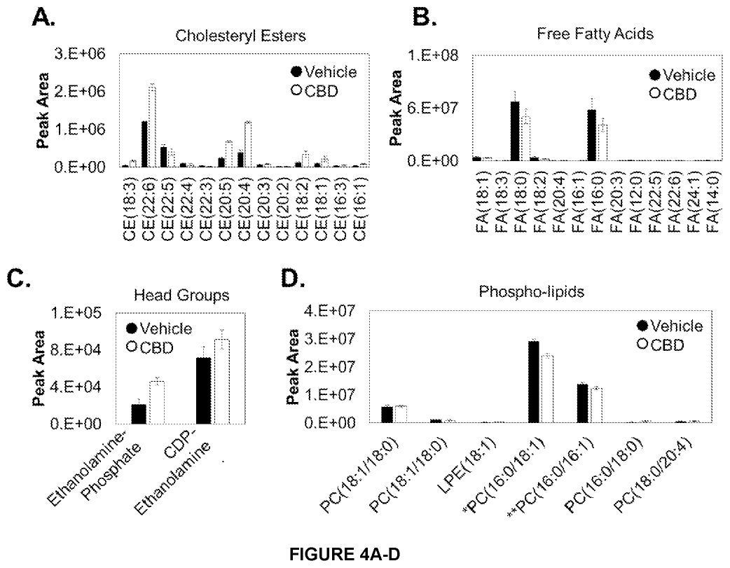

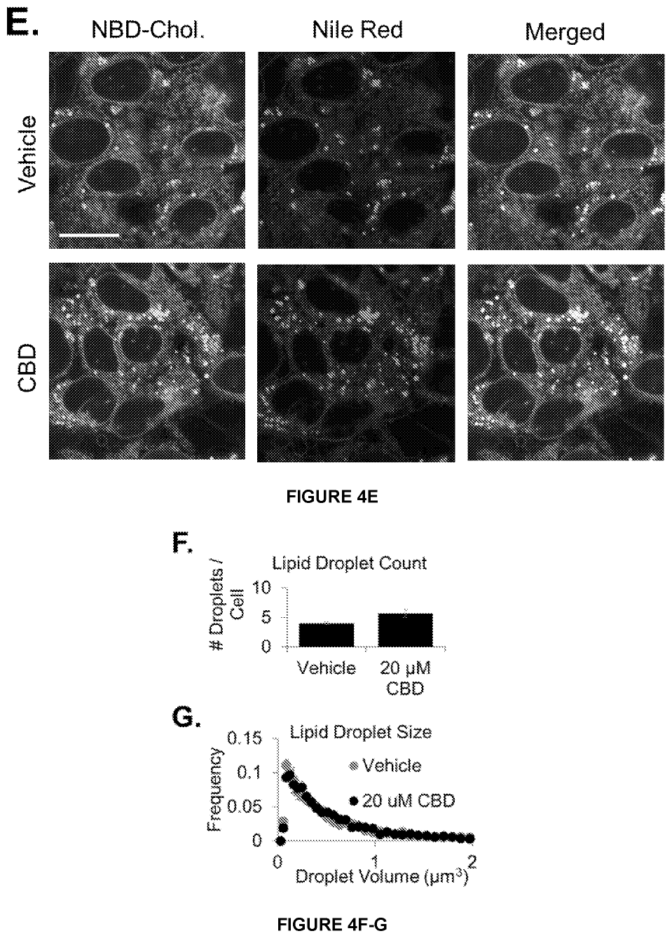

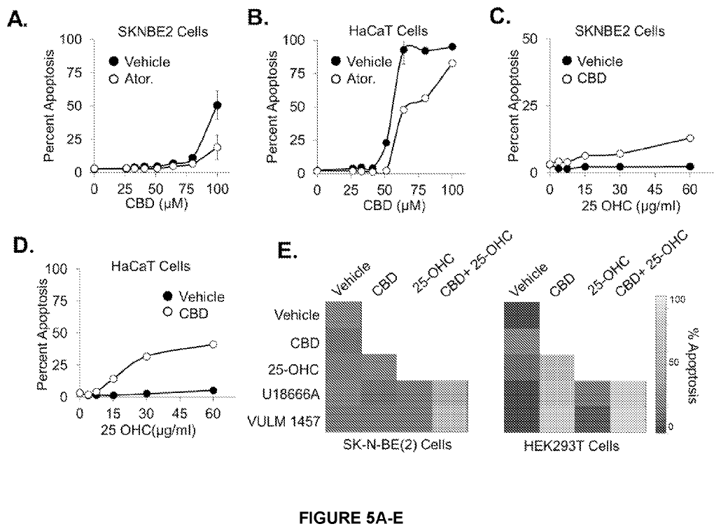

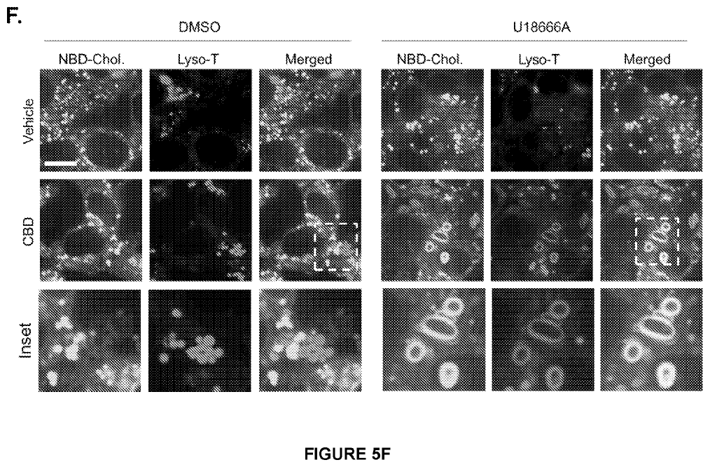

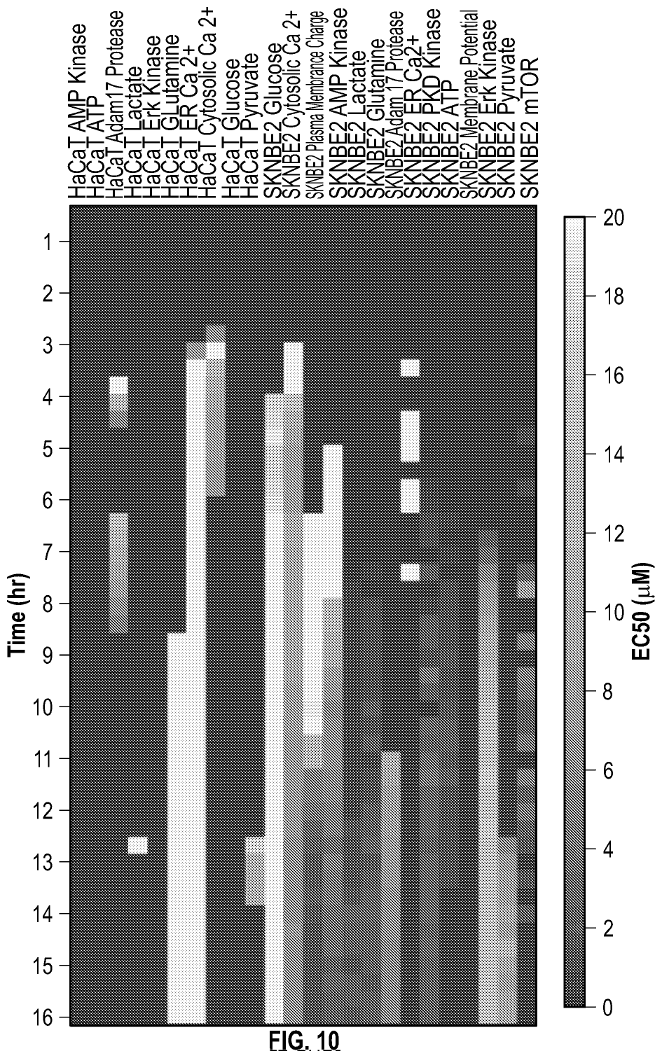

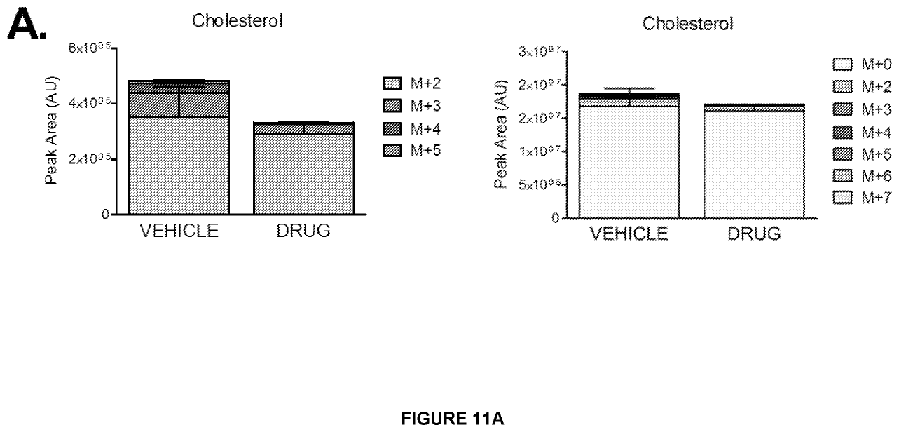

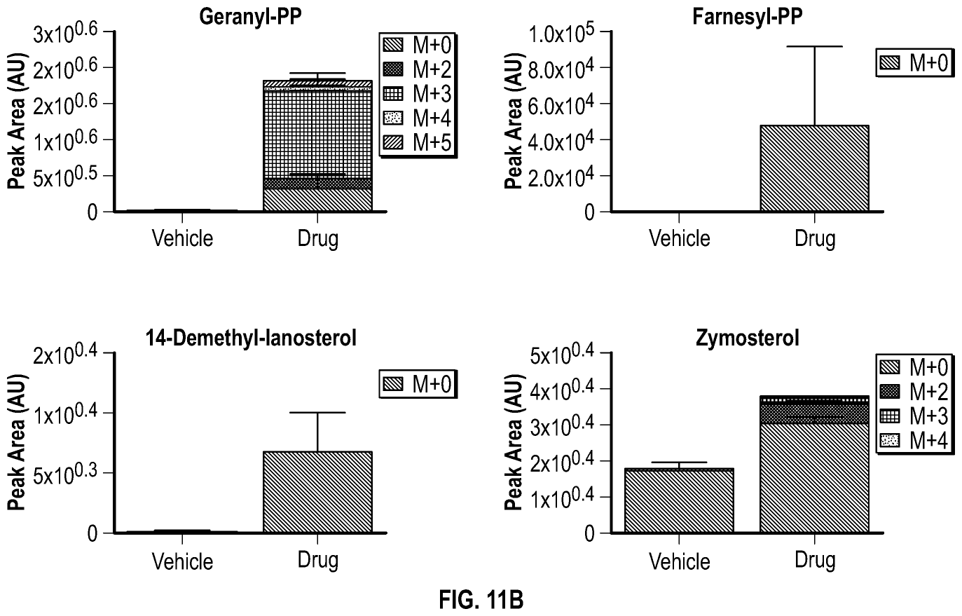

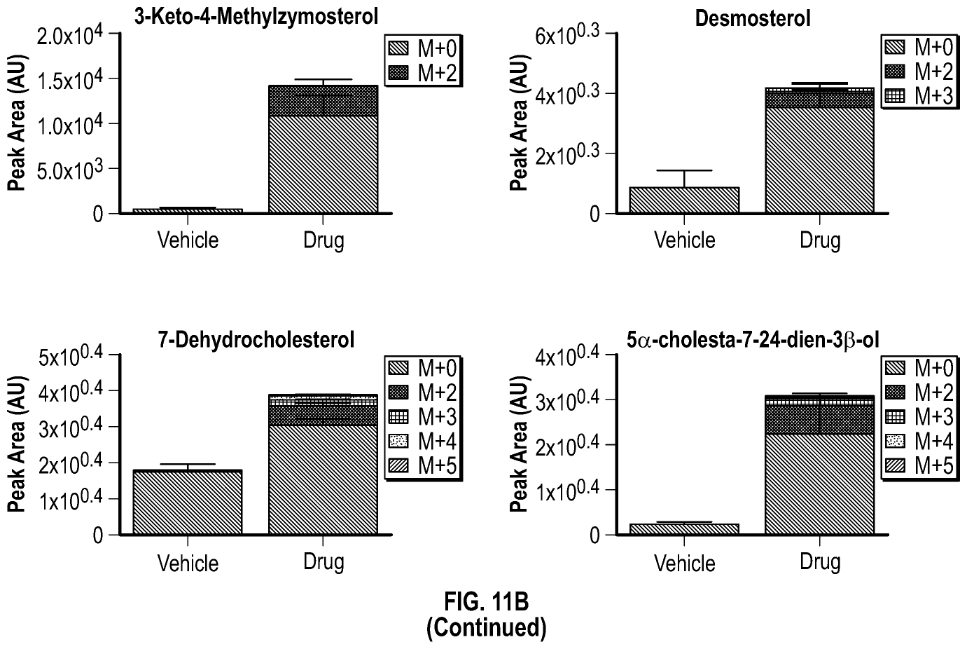

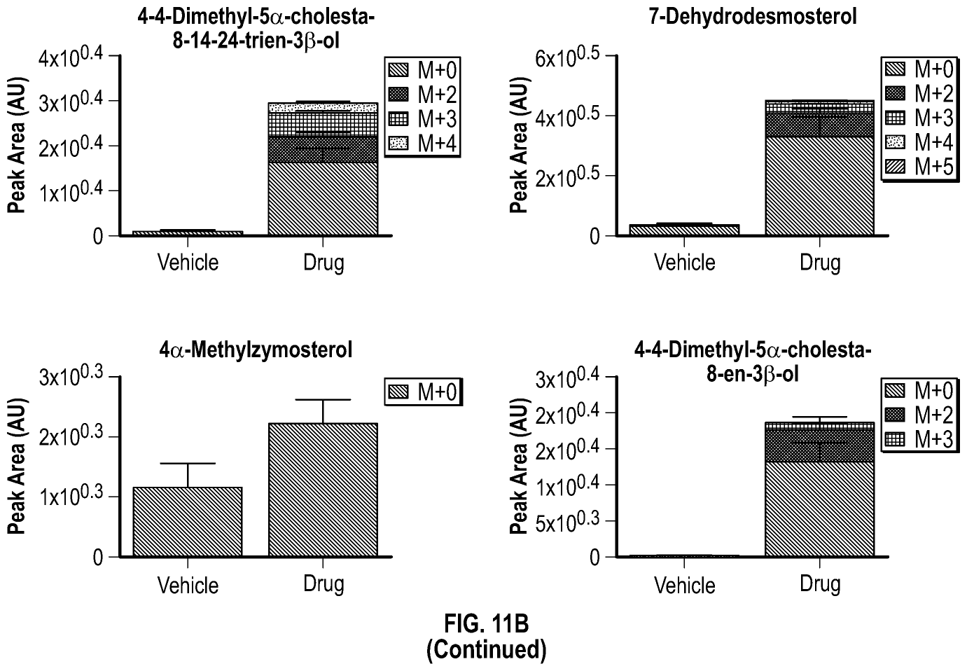

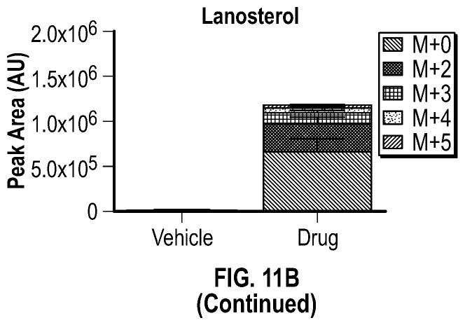

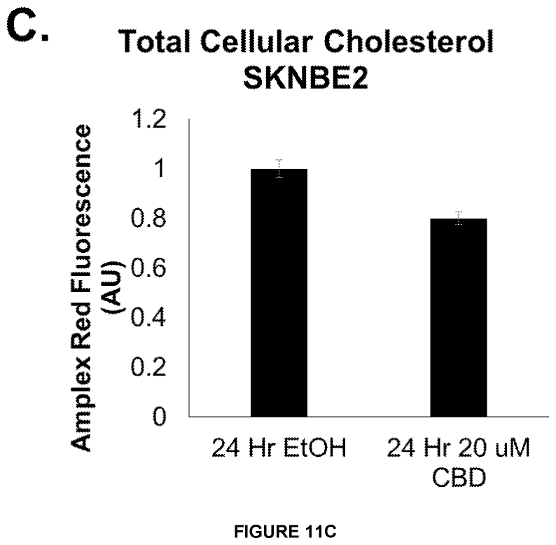

In one embodiment, the inventive technology demonstrates a multi-omics approach that provides an intrinsic cross-validation of CBD's ability to disrupt cholesterol homeostasis. Specifically, the present inventors have employed a strategy to integrate transcriptomics, metabolomics, and proteomics as a comprehensive tool to identify the biochemical components in the MoA of CBD in cells and further demonstrated consistent evidence that CBD disrupts cellular cholesterol homeostasis. Multiple aspects of cholesterol related cellular processes proved to be perturbed, including cholesterol biosynthesis (FIGS. 2D, 3B), transport (FIGS. 2D, 5E,F), and storage (FIGS. 4 A-G). All three-omics methods provided evidence for perturbed cholesterol biosynthesis. For instance, transcriptomics and proteomics reported transcriptional activation and protein accumulation of the rate-limiting enzyme in the biosynthetic pathway of cholesterol, HMG-CR (FIG. 2D). Increased HMG-CR protein production is a canonical response to decreased cholesterol levels in the ER, where cholesterol is sensed through the SREBP-SCAP axis. Consistent with this observation, the present inventors found within the CBD response that metabolic precursors of cholesterol are accumulating (FIGS. 3B, 11A) and total cholesterol production is decreasing (FIGS. 3B, 11A, 11B), demonstrating that the last step in cholesterol biosynthesis pathway is inhibited. Thus, in one preferred embodiment, CBD may trigger the sensing of low cholesterol via SREBP-SCAP processing, inhibition of the DHCR7/24 enzymatic step of cholesterol biosynthesis, and activation cholesterol esterification/storage, all within the ER.

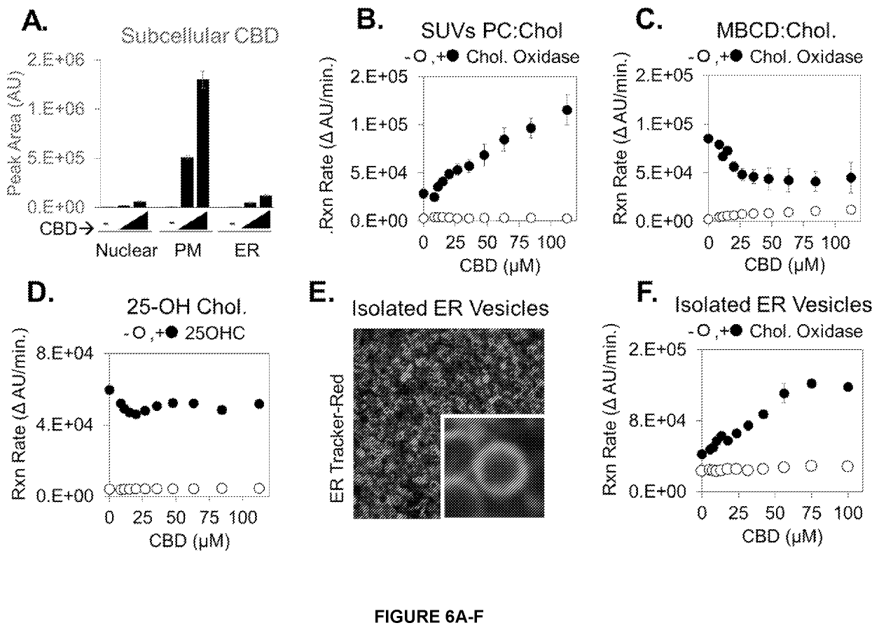

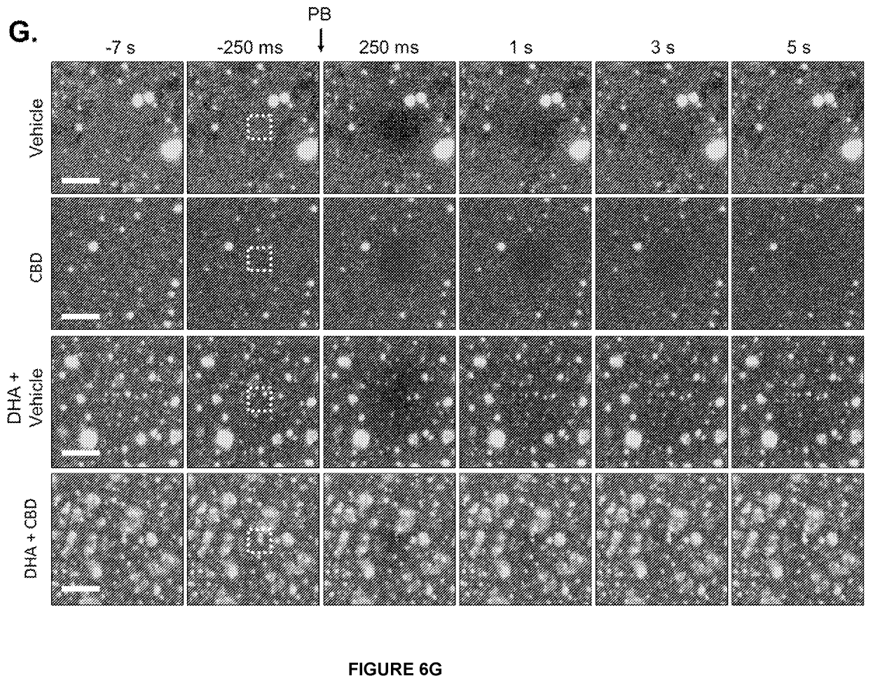

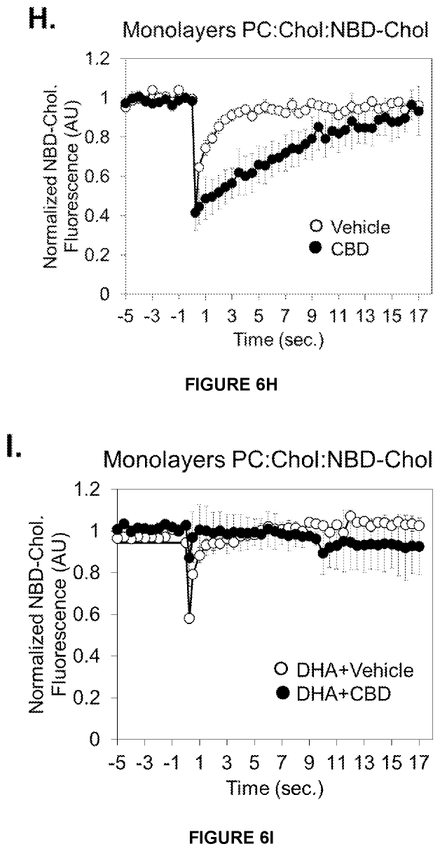

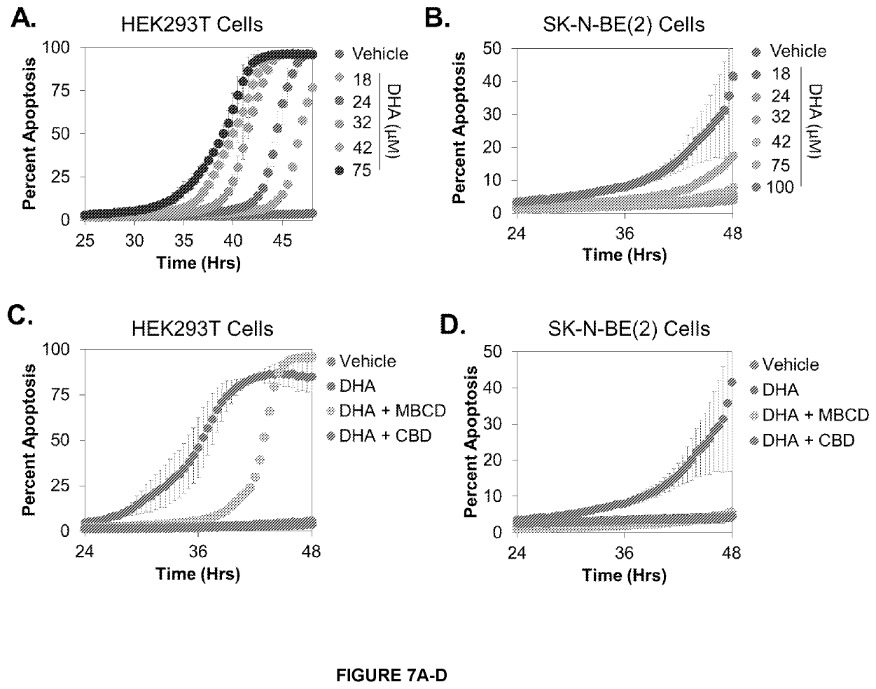

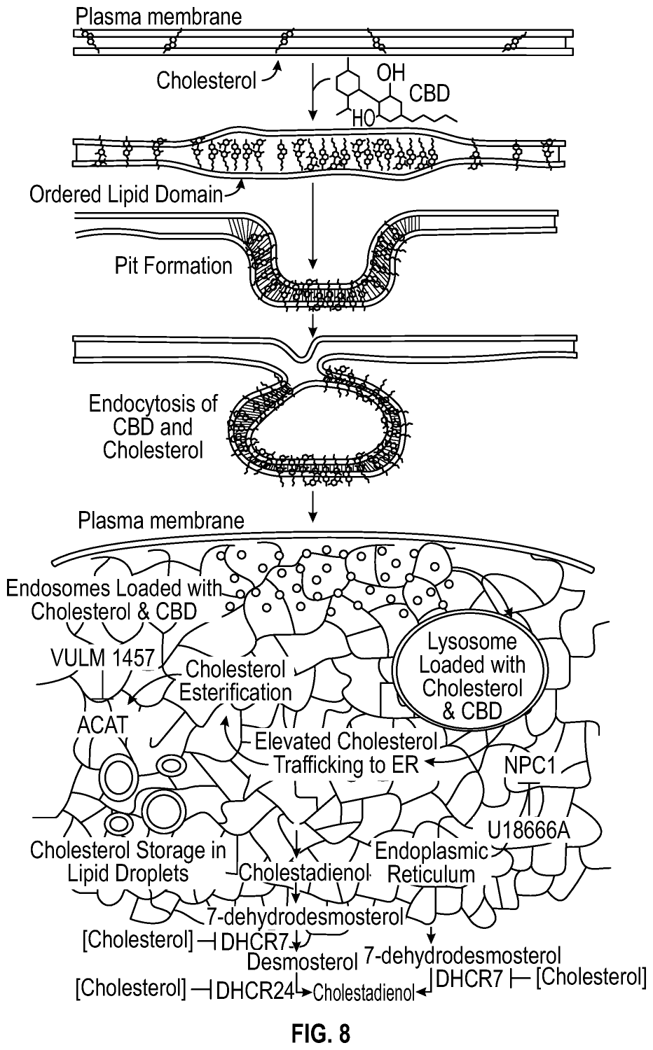

In one embodiment, the inventive technology demonstrates that CBD incorporates into cellular membranes and increases lipid order. Despite multiple lines of evidence in preclinical studies of CBD outlining a wide array of phenotypic effects, very little evidence has emerged to substantiate how these effects originate. The present inventor's data using PC:Chol SUVs shows that CBD incorporation into SUVs causes increased accessibility of cholesterol to cholesterol oxidase, presumably through tight packing of cholesterol, CBD and possibly PC (FIG. 6B). This result is surprising and totally unexpected because it suggests that CBD can induce ordered domains in the complete absence of sphingolipids, which are historically used in formulations of model membranes for studying lipid order. Nevertheless, the present inventors detected the same behavior of cholesterol in CBD treated ER membranes derived from subcellular fractionation of living cells (FIGS. 6 B, F), which demonstrates that this ordering effect can also occur in membranes of complex composition. Consistent with CBD's ability to constrain the cholesterol orientation and packing, the present inventors demonstrate that fluorescent cholesterol displays diminished lateral diffusion in response to CBD exposure to synthetic membranes, and that this affect opposes and can be reversed by the poly-unsaturated lipid DHA (FIGS. 6 G-I). Since diminished lateral diffusion of lipids has can be a hallmark of increased lipid order, this data represents evidence for CBD induced order in membranes. Additionally, the opposing effect between CBD and DHA on model membranes was recapitulated in living cells in an opposing effect on apoptosis (FIGS. 7 B, C), which strengthens the inventive technology's findings that CBD can affect the lipid order in cholesterol containing membranes in living cells.

The present inventors subcellular fractionation enabled a determination that CBD incorporates primarily into the plasma membrane of cells and to a lesser degree the membranes of the ER and the nuclear envelope (FIG. 6A), demonstrating that CBD itself may be trafficked with other cellular membrane components. Previous studies show that internalization of the plasma membrane through endocytosis is heavily dependent on the formation of ordered lipid domains, within which the protein machinery that mediates membrane curvature and vesicle pinching is assembled. This role for ordered lipid domains in endocytosis has been shown for both caveolin and clathrin-mediated pathways and can explain how the lipid ordering effect of CBD would result in increased intracellular transport of cholesterol from the plasma membrane.

In another embodiment, the inventive technology demonstrates that CBD dependent activation of lipid order causes downstream effects on cholesterol subcellular distribution, storage, and biosynthesis. An initial survey of cellular components affected by CBD using proteomics and transcriptomics yielded a surprisingly large number of affected components known to be involved in a fairly diverse array of biochemical pathways and cellular process, including translation, mitochondrial function and Cajal function (FIG. 2 D). These data and biosensor profiling data (FIG. 10) indicate the potential that either CBD has a diverse number of drug targets, or the CBD drug target itself interacts with many downstream pathways and processes, where in particular CBD's ability to influence cholesterol is an upstream effect in the CBD response.

As highlighted above, the apoptotic effect of CBD is heavily dependent on cellular cholesterol levels (FIGS. 5 A-D) suggesting a role for cholesterol in the upstream response. Additional lines of evidence revealed that CBD enhances the flux of fluorescent cholesterol through the lysosomal compartment (FIG. 5F), on its way to being stored in lipid droplets (FIGS. 4A, E-F). This flux of cholesterol was also shown to be related to CBD's ability to affect cellular apoptosis, as pharmacological inhibition of cholesterol export from the lysosome or inhibition of cholesterol esterification causes massive apoptotic responses in CBD treated cells but has no detectable effect on control cells (FIG. 5 E). This synergistic effect between inhibitors of cholesterol trafficking/storage and CBD could be greatly enhanced by additional treatment of 25-hydroxy cholesterol, supporting the model that CBD increases trafficking and storage of cholesterol in lipid droplets as a pro-survival response. Indeed, the key event that initiates apoptosis in conditions in which the inventor's observe CBD dependent cell death may be accumulation of cholesterol in either the ER or lysosome, where cholesterol is maintained at relatively low levels. Lysosomal accumulation of cholesterol has been demonstrated in previous studies to be a potent upstream trigger of apoptosis.

Additional embodiments demonstrate that the increased cholesterol transport from the lysosome to the ER can explain why biosynthesis of cholesterol is inhibited in the final steps catalyzed by DHCR24/DHCR7 via product inhibition of DHCR24 and DHCR7, which is known to occur for DHCR7. As lipid order has been a strong modulator of this endocytic flux of cholesterol containing vesicles in a multitude of previous studies, the present inventions shows that CBD's pharmacological effect on lipid order is the primary initiator of the observed homeostatic shift in cholesterol maintenance in CBD treated cells. Taken together, embodiments of the data demonstrate that endocytic flux of plasma membrane cholesterol is upstream of CBD's ability to activate cholesterol transport through the lysosome to the ER and lipid droplets, as well as CBD's ability to inhibit cholesterol biosynthesis.

In another embodiment, the inventive technology demonstrates that the effects on cholesterol homeostasis explain CBD's broad therapeutic ability and predict potential risks in CBD use. The invention identifies key mechanistic components in the CBD response, classifies those components into broad biological processes, and identifies CBD as an efficient modulator of lipid order from which the downstream effects originate. This insight of CBD MoA through lipid order modulation has broad implications on clarifying the mechanistic origins of the clinical effects of CBD in a wide array of diseases. CBD has been painted as a panacea in the supplement industry. Indeed, many of the diseases where CBD is implicated as a therapeutic are known to engage transmembrane proteins that have functions reported to be modulated through ordered lipid domain processes. Thus, the reported panacea like of CBD parallels the importance of cholesterol mediated processes within the progression of these diseases.

The current inventive technology includes methods and compositions to modulate cholesterol levels in blood serum, cellular membranes, or insoluble plaques in a subject. In one embodiment, the invention includes the administering of a therapeutically effective amount of a cannabinoid compound and/or a cannabinoid analog to a patient. In one preferred embodiment, the cannabinoid compound and/or a cannabinoid analog may lower the cholesterol content of an insoluble plaque in a tissue of a patient as a therapeutic means to dissolve and remove a plaque in diseases originating from cholesterol containing plaques, including but not limited to cardiovascular disease, and Alzheimer's disease. In another preferred embodiment, the cannabinoid compound and/or a cannabinoid analog may lower of the patient's level of blood cholesterol similar to the result that may be achieved through the administration of a statin or other cholesterol controlling medications.

This embodiment may be particularly suited for a patient that cannot be administered a traditional statin due to side-effects or other incompatible health concerns. As such, in one preferred embodiment, the invention includes administering of a therapeutically effective amount of a cannabinoid compound and/or a cannabinoid analog, such as CBD or a CBD analog, to a patient that cannot use or is not responsive to statin or other cholesterol controlling medications.

In this embodiment, the modulation of cholesterol in a patient may be accomplished through the administration of a therapeutically effective amount of a cannabidiol (CBD) compound and/or a CBD analog to a patient. In certain preferred embodiments, CBD analogs may include, but not be limited to: HU-308, o-1821, o-1602, and abnormal CBD among other identified herein. Such CBD may be isolated from a Cannabis plant extract or synthetically produced.

In another preferred embodiment, cannabinoids and/or cannabinoid analogs may be administered to a patient as an adjuvant with another cholesterol treatment. As such, embodiments of the invention further include novel systems, methods, and compositions for the modulation of cholesterol in a patient through the administration of a therapeutically effective amount (or effective amount) of a cannabinoid compound and/or a cannabinoid analog to a patient in combination with a separate cholesterol treatment. In this preferred embodiment, the modulation of cholesterol in a patient may be accomplished through the administration of a therapeutically effective amount of a cannabidiol (CBD) compound and/or a CBD analog to a patient in combination with, for example, a HMG-CoA reductase inhibitor, which may also be referred to as a statin.

Another embodiment of the current invention may include the use of one or more cannabinoid and/or cannabinoid analogs alone, or in combination with a cholesterol treatment. In one preferred embodiment, invention may include the use of one or more cannabinoids and/or cannabinoid analogs in combination with a non-statin cholesterol regulation medication, such as cholestyramine (Locholest®, Prevalite®, Questran®), colesevelam (WelChol®), and colestipol (Colestid®), clofibrate (Atromid-S®), fenofibrate (Antara®, Fenoglide®, Lipofen®, TriCor®, Triglide®, Trilipix®), and gemfibrozil (Lopid®) or Ezetimibe (Zetia®).

Another embodiment of the current invention may include novel systems, methods, and compositions to modulate cholesterol to treat one or more cholesterol-related conditions in a patient. Examples of such conditions may include, but not be limited to: CVD, diabetes, dyslipidemia, and other cholesterol-related conditions that may present in a patient.

Another embodiment of the invention includes modulating cholesterol sensing, production, storage and trafficking within cell, tissue, plaque and/or a patient. In certain embodiments, administration of one or more cannabinoid and/or cannabinoid analogs, such as CBD, may induce structural changes in cellular endoplasmic reticulum. In certain embodiments, administration of one or more cannabinoid and/or cannabinoid analogs may alter the solubility of cholesterol in aqueous bodily fluids. In certain embodiments, administration of one or more cannabinoid and/or cannabinoid analogs may lower the cholesterol content of intracellular and/or extracellular plaques.

Examples of cholesterol-related conditions include hypercholesterolemia, lipid disorders such as hyperlipidemia, and atherogenesis and its sequelae of cardiovascular diseases; including atherosclerosis, other vascular inflammatory conditions, myocardial infarction, ischemic stroke, occlusive stroke, dyslipidemia and peripheral vascular diseases, as well as other conditions in which decreasing cholesterol can produce a benefit. Other cholesterol-related conditions treatable with compositions, kits, and methods of the present invention include those currently treated with statins, as well as other conditions in which decreasing cholesterol absorption can produce a benefit.

In certain embodiments, a CBD or CBD analog of the present invention can be used to reduce cholesterol levels, in particular non-HDL plasma cholesterol levels, e.g. by reducing cholesterol absorption. In some preferred embodiments, a CBD or CBD analog and at least one other cholesterol modulating composition, such as preferably a statin, can be used to reduce cholesterol levels.

In one embodiment, the invention may include the administration of a therapeutically effective amount of a cannabinoid, and in particular a cannabidiol analog, to a patient in need thereof to treat a cholesterol related condition which may include the accumulation of a cholesterol containing plaque. Such therapeutic treatment may result in the removal or reduction of the size of a cholesterol containing plaque. In this embodiment, the removal or reduction of the size of said cholesterol containing plaque may be achieved by altering the solubility of cholesterol contained inside a plaque, or by altering the accessibility of cholesterol contained inside the plaque to one or more cholesterol transport protein. Alternatively, the removal or reduction of the size of said cholesterol containing plaque is achieved by altering the accessibility of cholesterol contained inside a plaque to one or more enzymes that utilize cholesterol as a reactant.

The removal or reduction of the size of said cholesterol containing plaque may be achieved by CBD or CBD analogs binding directly to cholesterol, which may further cholesterol alters the enzymatic activity of proteins that bind cholesterol. In such an embodiment, CBD or CBD analogs may bind directly to cholesterol such that it alters the enzymatic activity of proteins that use cholesterol as a reactant or product.

In related aspects, this disclosure also provides the use of CBD or a CBD analog, or a pharmaceutically acceptable salt thereof, in the manufacture of a medicament for the treatment of n cholesterol-related disorder, such as CVD, or diabetes. These methods of treatment may include the administration of a pharmaceutical composition described herein. Thus, this disclosure also provides pharmaceutical compositions comprising one or more CBD or CBD analog compounds of this disclosure useful in the methods of treatment of this disclosure, these pharmaceutical compositions or formulations may include a compound of this disclosure and a pharmaceutically acceptable carrier, diluent, or excipient. Administration may include CBD alone, and/or with another CBD analog, and/or with another cholesterol controlling compounds, such as a statin.

In certain embodiments, the cannabinoid is a natural cannabinoid. In certain embodiments, the cannabinoid is a natural cannabinoid found in a Cannabis plant. In certain embodiments, the cannabinoid is a synthetic cannabinoid. In certain embodiments, the cannabinoid is a mixture of natural cannabinoids. In certain embodiments, the cannabinoid is a mixture of synthetic cannabinoids. In certain embodiments, the cannabinoid is a mixture of natural and synthetic cannabinoids. The term “natural cannabinoid” as used herein generally refers to a cannabinoid which can be found in isolated from and/or extracted from a natural resource, such as plants. “Synthetic cannabinoids” are a class of chemicals that are different from the cannabinoids found e.g. in Cannabis but which also bind to cannabinoid receptors. The term “cannabinoid” as used herein generally refers to one of a class of diverse chemical compounds that act on a cannabinoid receptor in cells that repress neurotransmitter release in the brain. The term “cannabinoid” as used herein further refers a chemical compounds that acts on cannabinoid receptors or has a structure similar the stature of a compound acting on cannabinoid receptor in cells. Ligands for these receptor proteins include the endocannabinoids (produced naturally in the body by humans and animals), the phytocannabinoids (found in Cannabis and some other plants), and synthetic cannabinoids (manufactured artificially).

In certain embodiments, a cannabinoid may be selected from the group consisting of cannabidiol (CBD), cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabimol (iso-THC), cannabic yclol (CBL), cannabicitran (CBT), cannabivarin (CBV), tetrahydrocannabivarin (THCV), cannabidivarin (CBDV), cannabichromevarin (CBCV), cannabigerovarin (CBGV) and cannabigerol monomethyl ether (CBGM), salts thereof, derivatives thereof and mixtures of cannabinoids.

The terms “cannabidiol” and “CBD” are interchangeably used herein and refer to a non-psychotropic cannabinoid having structure as described in Formula I below, salt or derivatives thereof, such as A4-cannabidiol, A5-cannabidiol, A6-cannabidiol, A\hat{ }-cannabidiol, Δ1-cannabidiol A2-cannabidioli Δ3-cannabidiol.

The terms cannabinoid, and in particular CBD, and CBD analogs further include cannabinoid glycoside forms, acetylated forms, and acetylated cannabinoid forms, for example as described by Sayre et al. in U.S. Ser. Nos. 16/110,728 and 16/110,954, such glycoside and acetylated glycoside structures and their methods of production and bioconversion being incorporated by reference), which may have enhance bioavailability and act as a prodrug upon administration to a subject.

The term “prodrug” refers to a precursor of a biologically active pharmaceutical agent (drug). Prodrugs must undergo a chemical or a metabolic conversion to become a biologically active pharmaceutical agent. A prodrug can be converted ex vivo to the biologically active pharmaceutical agent by chemical transformative processes. In vivo, a prodrug is converted to the biologically active pharmaceutical agent by the action of a metabolic process, an enzymatic process or a degradative process that removes the prodrug moiety, such as a glycoside, to form the biologically active pharmaceutical agent.

In certain embodiments, a cannabinoid and/or cannabinoid analog may be selected from the group consisting of tetrahydrocannabinol, A9-tetrahydrocannabinol (THC), Δ8-tetrahydrocannabinol, standardized marijuana extracts, A8-tetrahydrocannabinol-DMH, Δ9-tetrahydrocannabinol propyl analog (THCV), 11-hydroxy-tetrahydrocannabinol, 1 1-nor-9-carboxy-tetrahydrocannabinol, 5′-azido-.A8-tetrahydrocannabinol, AMG-1 (CAS Number 205746-46-9), AMG-3 (CAS Number 205746-46-9), AM-411 (CAS Number 212835-02-4), (−)-1 1-hydroxy-7′-isothiocyanato-A8-THC (AM-708), (−)-1 1-hydroxy-7′-azido-A8-THC (AM-836), AM-855 (CAS Number 249888-50-4), AM-919 (CAS Number 164228-46-0), AM926, AM-938 (CAS Number 303113-08-8), cannabidiol (CBD), cannabidiol propyl analog (CBDV), cannabinol (CBN), cannabichromene, cannabichromene propyl analog, cannabigerol, CP 47,497 (CAS Number (1S,3R): 114753-51-4), CP 55,940 (CAS Number 83002-04-4), CP 55,244 (CAS Number 79678-32-3), CT-3 (ajulemic acid), dimethylheptyl HHC, HU-210 (1,1-Dimethylheptyl-11-hydroxy-tetrahydrocannabinol), HU-211 (CAS Number 112924-45-5), HU-308 (CAS Number 1220887-84-2), WIN 55212-2 (CAS Number 131543-22-1), desacetyl-L-nantradol, dexanabinol, JWH-051 (Formula C25H3802), levonantradol, L-759633 (Formula C26H40O2), nabilone, 0-1184, and mixtures thereof.

As used herein, o-1821 means the compound having the formula: 5Z, 8Z, 11Z, 14Z)-20-cyano-N-[(2R)-1hidroxipropan-2-yl]-16.16-dimetilicosa5,8,11,14-tetraenamida.

As used herein, o-1602 means the compound having the formula: 5-methyl-4-[(1R,6R)-3-methyl-6-(1-methylethenyl)-2-cyclohexen-1-yl]-1,3-benzenediol.

As used herein, means the compound having the formula: HU-308 4-[4-(1,1-dimethylheptyl)-2,6-dimethoxyphenyl]-6,6-dimethyl-bicyclo[3.1.1]hept-2-ene-2-methanol

As used herein, abnormal CBD means the compound having the formula: 4-[(1R,6R)-3-Methyl-6-(1-methylethenyl)-2-cyclohexen-1-yl]-5-pentyl-1,3-benzenediol.

In certain embodiment, a cannabinoid, such as CBD or a cannabinoid analog may be derived from a plant extract or chemically synthesized. In other embodiment, one or more a cannabinoids, such as CBD or a cannabinoid analog may be isolated and/or purified from the entourage of cannabinoids in a natural plant extract. The terms “isolated”, “purified”, or “biologically pure” as used herein, refer to material that is substantially or essentially free from components that normally accompany the material in its native state or when the material is produced.

The term “lipid-order disease condition” or “lipid order disease” refers to a disease condition, preferably in a human that may treated through the increasing of the lipid order in the cell membranes of the subject's cells. Examples, may include Alzheimer's disease (AD), Huntington's disease (HD), and Parkinson's disease (PD), and even to the cognitive deficits typical of the old age. Such increases of lipid-order may include increasing the number and/or integrity of lipid rafts. Lipid rafts are portions of the plasma membrane that contain nano/micro-domains that are enriched in cholesterol. These rafts are thought to represent highly dynamic structures dispersed throughout the membrane of cells that recruit downstream signaling molecules upon activation by external or internal signals. Rafts also contain a high amount of sphingomyelin, which is enriched in the outer leaflet of the plasma membrane, indicating that some trans-bilayer translocation must occur to form and stabilize these domains. In neurons, membrane rafts have been detected at synapses, where they are thought to contribute to pre- and postsynaptic function. In another example, lipid rafts promote interaction of the amyloid precursor protein (APP) with the secretase (BACE-1) responsible for generation of the amyloid β peptide, Aβ. Rafts also regulate cholinergic signaling as well as acetylcholinesterase and Aβ interaction. In addition, such major lipid raft components as cholesterol and GM1 ganglioside have been directly implicated in pathogenesis of the disease. Perturbation of lipid raft integrity can also affect various signaling pathways leading to cellular death and AD. Moreover, it has been shown that the autophagic-lysosomal pathway is aberrant in Alzheimer's disease brain. However, lipid rafts that mediate amyloid precursor protein can disturb autophagy, thus blocking the autophagic-lysosomal pathway and aggravating the disease. Therefore, we speculate that in Alzheimer's disease, lipid rafts play an important role in blocking the autophagic-lysosomal pathway of amyloid precursor protein processing and amyloid-beta degradation, thereby exacerbating the progression of this disease. In one embodiment, increasing lipid-order may also include increasing the number and/or integrity of lipid raft in a cell membrane. Increasing “lipid order” may further alter the constituency and/or components of the order domain in the cell membrane.

The term “compound(s)” and equivalent expressions, are meant to embrace compounds herein described, which expression includes the prodrugs, the pharmaceutically acceptable salts, the oxides, and the solvates, e.g. hydrates, where the context so permits.

The term “method of treating” means amelioration or relief from the symptoms and/or effects associated with the diseases or disorders described herein.

Alternatively the CBD or any other cannabinoid is/are in a substantially pure or isolated form. A “substantially pure” or “isolated” preparation of cannabinoid is defined as a preparation having a chromatographic purity (of the desired cannabinoid) of greater than 90%, more preferably greater than 95%, more preferably greater than 96%, more preferably greater than 97%, more preferably greater than 98%, more preferably greater than 99% and most preferably greater than 99.5%, as determined by area normalization of an HPLC profile or other similar detection method. Preferably the substantially pure cannabinoid used in the invention is substantially free of any other naturally occurring or synthetic cannabinoids, including cannabinoids which occur naturally in Cannabis plants which are not intended to be administered to a subject. In this context “substantially free” can be taken to mean that no cannabinoids other than the target cannabinoid are detectable by HPLC or other similar detection method.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. Moreover, “treatment”, as used herein, covers any treatment of a disease in a mammal, and particularly in a human, and includes: (a) preventing the disease from occurring in a subject which may be predisposed to the disease but has not yet been diagnosed as having it: (b) inhibiting the disease, i.e., arresting its development; (c) relieving the disease, i.e., causing regression of the disease; (d) protection from or relief of a symptom or pathology caused by or related to cholesterol: storage, transport, biosynthesis, metabolism, flux, orientation, and/or lipid order; (e) reduction, decrease, inhibition, amelioration, or prevention of onset, severity, duration, progression, frequency or probability of one or more symptoms or pathologies associated with cholesterol: storage, transport, biosynthesis, metabolism, flux, orientation, and/or lipid order; and (f) prevention or inhibition of a worsening or progression of symptoms or pathologies associated with cholesterol: storage, transport, biosynthesis, metabolism, flux, orientation, and/or lipid order.

Compounds described herein may contain one or more asymmetric centers and may thus give rise to enantiomers, diastereomers, and other stereoisomeric forms. Each chiral center may be defined, in terms of absolute stereochemistry, as (R)- or (S)-. The present invention is meant to include all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

As would be understood by a person of ordinary skill in the art, the recitation of “a compound” is intended to include salts, solvates, oxides, and inclusion complexes of that compound as well as any stereoisomeric form, or a mixture of any such forms of that compound in any ratio. Thus, in accordance with some embodiments of the invention, a compound as described herein, including in the contexts of pharmaceutical compositions, methods of treatment, and compounds per se, is provided as the salt form.

References to CBD, and CBD-analogs or derivatives thereof, particularly with regard to therapeutic use, will be understood to also encompass pharmaceutically acceptable salts of such compounds. The term “pharmaceutically acceptable salt” refers to salts prepared from pharmaceutically acceptable non-toxic acids or bases including inorganic acids and bases and organic acids and bases. Examples of pharmaceutically acceptable salts include, but are not limited to, mineral or organic acid salts of basic residues such as amines, or alkali or organic salts of acidic residues such as carboxylic acids. Pharmaceutically-acceptable salts include the conventional non-toxic salts or the quaternary ammonium salts of the parent compound formed, for example, from non-toxic inorganic or organic acids. Such conventional nontoxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamolc, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Pharmaceutically acceptable salts are those forms of agents, suitable for use in contact with the tissues of human beings and animals without excessive toxicity, irritation, allergic response, or other problem or complication, commensurate with a reasonable benefit/risk ratio.

Pharmaceutically-acceptable salt forms may be synthesized from agents which contain a basic or acidic moiety by conventional chemical methods. Generally, such salts are, for example, prepared by reacting the free acid or base forms of these agents with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in at page 1418 of Remington's Pharmaceutical Sciences, 17th ed., Mack Publishing Company, Easton, Pa., 1985.

The term “pharmaceutically acceptable” means it is, within the scope of sound medical judgment, suitable for use in contact with the cells of humans and lower animals without undue toxicity, irritation, allergic response and the like, and are commensurate with a reasonable benefit/risk ratio. Such pharmaceutical compositions/formulations are useful for administration to a subject, in vivo or ex vivo. Pharmaceutical compositions and formulations include carriers or excipients for administration to a subject.

As used herein the terms “pharmaceutically acceptable” and “physiologically acceptable” further mean a biologically compatible formulation, gaseous, liquid or solid, or mixture thereof, which is suitable for one or more routes of administration, in vivo delivery or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions. The formulations may, for convenience, be prepared or provided as a unit dosage form. In general, formulations are prepared by uniformly and intimately associating the active ingredient with liquid carriers or finely divided solid carriers or both, and then, if necessary, shaping the product. For example, a tablet may be made by compression or molding. Compressed tablets may be prepared by compressing, in a suitable machine, an active ingredient in a free-flowing form such as a powder or granules, optionally mixed with a binder, lubricant, inert diluent, preservative, surface-active or dispersing agent. Molded tablets may be produced by molding, in a suitable apparatus, a mixture of powdered compound moistened with an inert liquid diluent. The tablets may optionally be coated or scored and may be formulated so as to provide a slow or controlled release of the active ingredient therein.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone. Supplementary active compounds (e.g., preservatives, antioxidants, antimicrobial agents including biocides and biostats such as antibacterial, antiviral and antifungal agents) can also be incorporated into the compositions. Preservatives and other additives include, for example, antimicrobials, anti-oxidants, chelating agents and inert gases (e.g., nitrogen). Pharmaceutical compositions may therefore include preservatives, antimicrobial agents, anti-oxidants, chelating agents and inert gases.

Preservatives can be used to inhibit microbial growth or increase stability of the active ingredient thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

Pharmaceutical compositions can optionally be formulated to be compatible with a particular route of administration. Exemplary routes of administration include administration to a biological fluid, an immune cell (e.g., T or B cell) or tissue, mucosal cell or tissue (e.g., mouth, buccal cavity, labia, nasopharynx, esophagus, trachea, lung, stomach, small intestine, vagina, rectum, or colon), neural cell or tissue (e.g., ganglia, motor or sensory neurons) or epithelial cell or tissue (e.g., nose, fingers, ears, cornea, conjunctiva, skin or dermis). Thus, pharmaceutical compositions include carriers (excipients, diluents, vehicles or filling agents) suitable for administration to any cell, tissue or organ, in vivo, ex vivo (e.g., tissue or organ transplant) or in vitro, by various routes and delivery, locally, regionally or systemically.

Exemplary routes of administration for contact or in vivo delivery which a CBD and/or CBD analog can optionally be formulated include inhalation, respiration, intubation, intrapulmonary instillation, oral (buccal, sublingual, mucosal), intrapulmonary, rectal, vaginal, intrauterine, intradermal, topical, dermal, parenteral (e.g., subcutaneous, intramuscular, intravenous, intradermal, intraocular, intratracheal and epidural), intranasal, intrathecal, intraarticular, intracavity, transdermal, iontophoretic, ophthalmic, optical (e.g., corneal), intraglandular, intraorgan, and intralymphatic.

Administering of compounds and/or pharmaceutical compositions in “therapeutically effective amount,” or “effective amount” to a subject may involve administering therapeutically effective amounts, which means an amount of compound effective in treating the stated conditions and/or disorders in a subject. In on preferred embodiment, a “therapeutically effective amount,” or “effective amount” may cause a modulation in cholesterol in a subject that is beneficial to that subject. This modulation may include a decrease in the amount of cholesterol in the subject's cells, tissues, plasma and other biological fluids. This modulation may also include a decrease in the amount of plaques in a subject, for example cholesterol plaques in an arterial wall in a subject. This modulation may also include an increase in lipid order in cholesterol containing lipid membranes of a cell in a subject. This modulation may also include activation of cholesterol transport in the cells of a subject. This modulation may also include activation of cholesterol storage in the cells of a subject. This modulation may also include inhibition of cholesterol biosynthesis in a subject. This modulation may also include enhanced flux of cholesterol through the lysosomal compartment. This modulation may also include the accumulation of metabolic precursors of cholesterol in a subject. This modulation may also include orientation of cholesterol in a cell membrane in a subject. This modulation may also include increasing cellular concentration of cholesterol that may form an inhibitory feedback loop on downstream cholesterol biosynthesis. Such modulation may be in vivo, ex vivo or in vitro.

In one embodiment, a therapeutically effective amount of a compound may be such that the subject receives a dosage of less than 0.1 μg/kg body weight/day to about 1000 mg/kg body weight/day, for example, a dosage of about 1 μg/kg body weight/day to about 1000 μg/kg body weight/day, such as a dosage of about 5 μg/kg body weight/day to about 500 μg/kg body weight/day, for example, a dosage of more than 1000 mg/kg body weight/day, for example, a dosage of less than 1000 mg/kg body weight/day, for example, a dosage of more than 1000 μg/kg body weight/day, for example, a dosage of less than 1000 μg/kg body weight/day.

Formulations suitable for parenteral administration include aqueous and non-aqueous solutions, suspensions or emulsions of the compound, which may include suspending agents and thickening agents, which preparations are typically sterile and can be isotonic with the blood of the intended recipient. Non-limiting illustrative examples of aqueous carriers include water, saline (sodium chloride solution), dextrose (e.g., Ringer's dextrose), lactated Ringer's, fructose, ethanol, animal, vegetable or synthetic oils. Examples of non-aqueous solvents are propylene glycol, polyethylene glycol, vegetable oils such as olive oil, and injectable organic esters such as ethyl oleate. Intravenous vehicles include fluid and nutrient replenishers, electrolyte replenishers (such as those based on Ringer's dextrose). The formulations may be presented in unit-dose or multi-dose kits, for example, ampules and vials, and may be stored in a freeze-dried (lyophilized) condition requiring addition of a sterile liquid carrier, for example, water for injections, prior to use.

For transmucosal or transdermal administration (e.g., topical contact), penetrants can be included in the pharmaceutical composition. Penetrants are known in the art, and include, for example, for transmucosal administration, detergents, bile salts, and fusidic acid derivatives. For transdermal administration, the active ingredient can be formulated into aerosols, sprays, ointments, salves, gels, pastes, lotions, oils or creams as generally known in the art.

For topical administration, for example, to skin, pharmaceutical compositions typically include ointments, creams, lotions, pastes, gels, sprays, aerosols or oils. Carriers which may be used include Vaseline, lanolin, polyethylene glycols, alcohols, transdermal enhancers, and combinations thereof. An exemplary topical delivery system is a transdermal patch containing an active ingredient.

For oral administration, pharmaceutical compositions include capsules, cachets, lozenges, tablets or troches, as powder or granules. Oral administration formulations also include a solution or a suspension (e.g., aqueous liquid or a non-aqueous liquid; or as an oil-in-water liquid emulsion or a water-in-oil emulsion).

For airway or nasal administration, pharmaceutical compositions can be formulated in a dry powder for delivery, such as a fine or a coarse powder having a particle size, for example, in the range of 20 to 500 microns which is administered in the manner by inhalation through the airways or nasal passage. Depending on delivery device efficiency, effective dry powder dosage levels typically fall in the range of about 10 to about 100 mg. Appropriate formulations, wherein the carrier is a liquid, for administration, as for example, a nasal spray or as nasal drops, include aqueous or oily solutions of the active ingredient.

For airway or nasal administration, aerosol and spray delivery systems and devices, also referred to as “aerosol generators” and “spray generators,” such as metered dose inhalers (MDI), nebulizers (ultrasonic, electronic and other nebulizers), nasal sprayers and dry powder inhalers can be used. MDIs typically include an actuator, a metering valve, and a container that holds a suspension or solution, propellant, and surfactant (e.g., oleic acid, sorbitan trioleate, lecithin). Activation of the actuator causes a predetermined amount to be dispensed from the container in the form of an aerosol, which is inhaled by the subject. MDIs typically use liquid propellant and typically, MDIs create droplets that are 15 to 30 microns in diameter, optimized to deliver doses of 1 microgram to 10 mg of a therapeutic. Nebulizers are devices that turn medication into a fine mist inhalable by a subject through a face mask that covers the mouth and nose. Nebulizers provide small droplets and high mass output for delivery to upper and lower respiratory airways. Typically, nebulizers create droplets down to about 1 micron in diameter.

Dry-powder inhalers (DPI) can be used to deliver the compounds of the invention, either alone or in combination with a pharmaceutically acceptable carrier. DPIs deliver active ingredient to airways and lungs while the subject inhales through the device. DPIs typically do not contain propellants or other ingredients, only medication, but may optionally include other components. DPIs are typically breath-activated, but may involve air or gas pressure to assist delivery.

For rectal administration, pharmaceutical compositions can be included as a suppository with a suitable base comprising, for example, cocoa butter or a salicylate. For vaginal administration, pharmaceutical compositions can be included as pessaries, tampons, creams, gels, pastes, foams or spray formulations containing in addition to the active ingredient a carrier, examples of appropriate carriers which are known in the art.

Pharmaceutical formulations and delivery systems appropriate for the compositions and methods of the invention are known in the art (see, e.g., Remington: The Science and Practice of Pharmacy (2003) 20.sup.th ed., Mack Publishing Co., Easton, Pa.; Remington's Pharmaceutical Sciences (1990) 18.sup.th ed., Mack Publishing Co., Easton, Pa.; The Merck Index (1996) 12.sup.th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11.sup.th ed., Lippincott Williams & Wilkins, Baltimore, Md.; and Poznansky et al., Drug Delivery Systems (1980), R. L. Juliano, ed., Oxford, N.Y., pp. 253-315).

The cholesterol modulating compounds of the invention may be packaged in unit dosage forms for ease of administration and uniformity of dosage. A “unit dosage form” as used herein refers to a physically discrete unit suited as unitary dosages for the subject to be treated; each unit containing a predetermined quantity of compound optionally in association with a pharmaceutical carrier (excipient, diluent, vehicle or filling agent) which, when administered in one or more doses, is calculated to produce a desired effect (e.g., prophylactic or therapeutic effect or benefit). Unit dosage forms can contain a daily dose or unit, daily sub-dose, or an appropriate fraction thereof, of an administered compound. Unit dosage forms also include, for example, capsules, troches, cachets, lozenges, tablets, ampules and vials, which may include a composition in a freeze-dried or lyophilized state; a sterile liquid carrier, for example, can be added prior to administration or delivery in vivo. Unit dosage forms additionally include, for example, ampules and vials with liquid compositions disposed therein. Unit dosage forms further include compounds for transdermal administration, such as “patches” that contact with the epidermis of the subject for an extended or brief period of time. The individual unit dosage forms can be included in multi-dose kits or containers. Pharmaceutical formulations can be packaged in single or multiple unit dosage forms for ease of administration and uniformity of dosage.

In the methods of the invention, the cholesterol modulating compounds of the invention may be administered in accordance with the methods at any frequency as a single bolus or multiple dose e.g., one, two, three, four, five, or more times hourly, daily, weekly, monthly or annually or between about 1 to 10 days, weeks, months, or for as long as appropriate. Exemplary frequencies are typically from 1-7 times, 1-5 times, 1-3 times, 2-times or once, daily, weekly or monthly. Timing of contact, administration ex vivo or in vivo delivery can be dictated by the symptom, pathology or adverse side effect to be treated.

Doses may vary depending upon whether the treatment is therapeutic or prophylactic, the onset, progression, severity, frequency, duration, probability of or susceptibility of the symptom to which treatment is directed, clinical endpoint desired, previous, simultaneous or subsequent treatments, general health, age, gender or race of the subject, bioavailability, potential adverse systemic, regional or local side effects, the presence of other disorders or diseases in the subject, and other factors that will be appreciated by the skilled artisan (e.g., medical or familial history). Dose amount, frequency or duration may be increased or reduced, as indicated by the clinical outcome desired, status of the infection, reactivation, pathology or symptom, or any adverse side effects of the treatment or therapy. The skilled artisan will appreciate the factors that may influence the dosage, frequency and timing required to provide an amount sufficient or effective for providing a prophylactic or therapeutic effect or benefit.

Another aspect of this disclosure provides pharmaceutical kits containing a pharmaceutical composition of this disclosure, prescribing information for the composition, and a container.

Such amounts generally vary according to a number of factors well within the purview of ordinarily skilled artisans. These include, without limitation: the particular subject, as well as its age, weight, height, general physical condition, and medical history, the particular compound used, as well as the carrier in which it is formulated and the route of administration selected for it; and, the nature and severity of the condition being treated. Further, in this context, the term “effective” is to be understood broadly to include reducing or alleviating the signs or symptoms of CVD, a cholesterol-related condition, reducing and/or otherwise modulating a level of cholesterol in a patient, or improving the clinical course of the same.

Administering typically involves administering pharmaceutically acceptable dosage forms, which means dosage forms of compounds described herein, and includes, for example, tablets, dragees, powders, elixirs, syrups, liquid preparations, including suspensions, sprays, inhalants tablets, lozenges, emulsions, solutions, granules, capsules, and suppositories, as well as liquid preparations for injections, including liposome preparations. Techniques and formulations generally may be found in Remington's Pharmaceutical Sciences, Mack Publishing Co., Easton, Pa., latest edition, which is hereby incorporated by reference in its entirety.

Administering may be carried out orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes. Compounds may be administered alone or with suitable pharmaceutical carriers, and can be in solid or liquid form, such as tablets, capsules, powders, solutions, suspensions, or emulsions.

Diseases or disorders amenable to the treatment method of the present invention include, without limitation, cardiovascular disease, peripheral arterial disease, diabetes, stroke, and dyslipidemia, among others.

The term “modulating” as used herein, may include increasing, or decreasing the level of one or more types of cholesterol. In certain embodiments, the term “cholesterol” includes both ester type cholesterol and free cholesterol. The term “cholesterol” may include low-density lipoprotein (LDL), high-density lipoprotein (HDL), very-low-density lipoprotein (VLDL), Chylomicrons (CM), and triglycerides.

As used herein, the terms “individual,” “subject,” and “patient,” used interchangeably herein, refer to a mammal, including, but not limited to, murines, simians, humans, mammalian farm animals, mammalian sport animals, and mammalian pets. Preferably, the subject herein is human. As further used herein, the terms “individual,” “subject,” and “patient,” includes “a subject or patient who has a cholesterol-related condition” and “a cholesterol-related condition patient or subject” “a cholesterol sensitive patient” “a patient in need of cholesterol-related therapy” “person with CVD or predisposed to CVD” are intended to refer to subjects who have been diagnosed with cancer, have received cholesterol-related therapy, are currently receiving cholesterol-related therapy, may receive cholesterol-related therapy in the future.

The terminology used herein is for describing embodiments and is not intended to be limiting. As used herein, the singular forms “a,” “and” and “the” include plural referents, unless the content and context clearly dictate otherwise. Thus, for example, a reference to “a CBD analog” may include a one, or combination of two or more CBD analogs. Unless defined otherwise, all scientific and technical terms are to be understood as having the same meaning as commonly used in the art to which they pertain. In addition, the term “including” as used herein to mean, and is used interchangeably with, the phrase “including but not limited to”. The term “or” is used herein to mean, and is used interchangeably with, the term “and/or,” unless context clearly indicates otherwise.

This disclosure is not limited to particular embodiments described as such may vary. The terminology used herein is for the purpose of describing particular embodiments only and is not intended to be limiting.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

The term “compound” or “compositions” “a compound of the invention” includes all solvates, complexes, polymorphs, radiolabeled derivatives, tautomer, stereoisomers, and optical isomers of the compounds of the CBD and it analogs generally described herein, and salts thereof, unless otherwise specified.

“Pharmaceutical compositions,” or “pharmaceutical compounds” are compositions that include an amount (for example, a unit dosage) of one or more of the disclosed compounds together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, Pa. (19th Edition).

The terms “pharmaceutically acceptable salt or ester” refers to salts or esters prepared by conventional means that include salts, e.g., of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid, and the like.

For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

The pharmaceutically acceptable acid and base addition salts as mentioned above are meant to comprise the therapeutically active non-toxic acid and base addition salt forms which the compounds can form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic, and like acids. Conversely, these salt forms can be converted into the free base form by treatment with an appropriate base.

The compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine, and the like.

Some of the compounds described herein may also exist in their tautomeric form.

Each publication or patent cited herein is incorporated herein by reference in its entirety.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### Examples

### Example 1: Chemical Analogs of Cannabidiol (CBD) Elicit Biochemical Responses in Mammalian Cells Similar to the Cholesterol Drugs

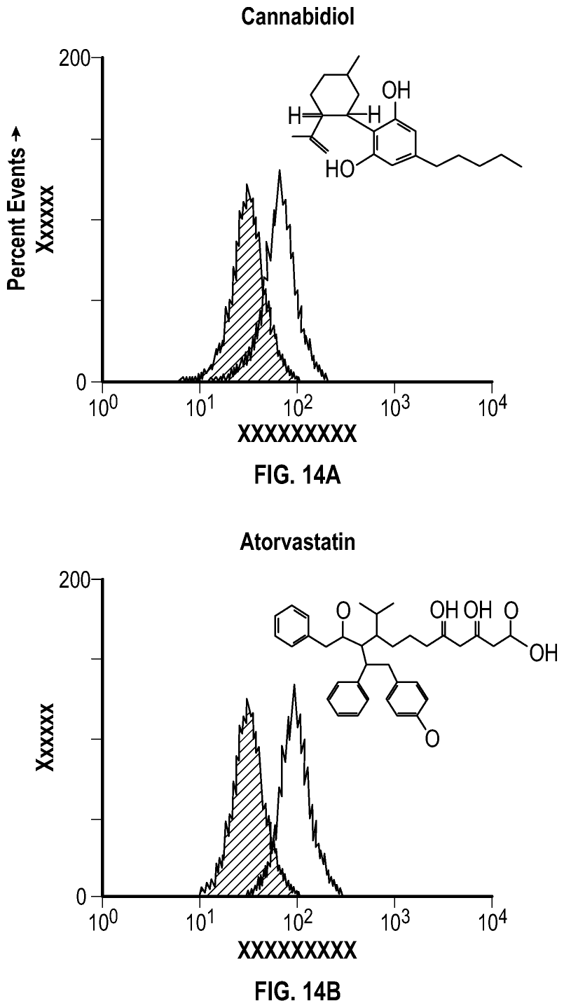

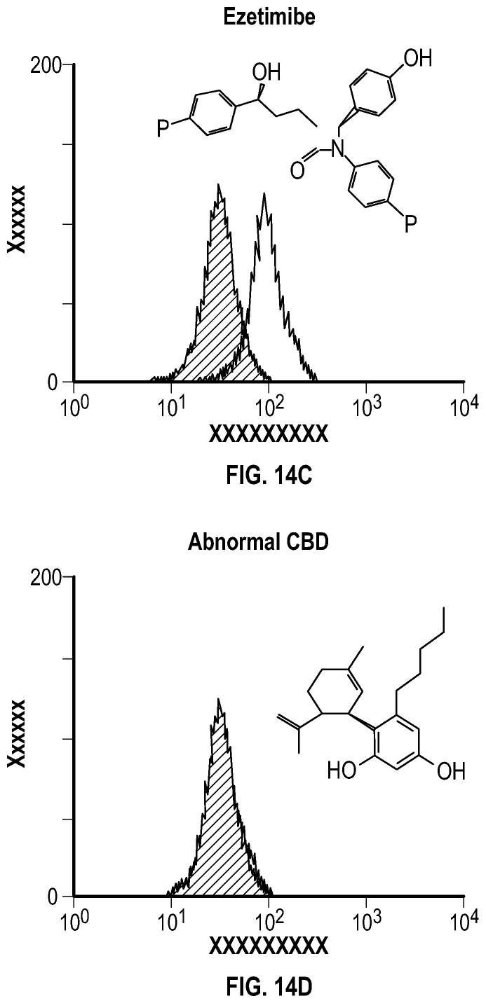

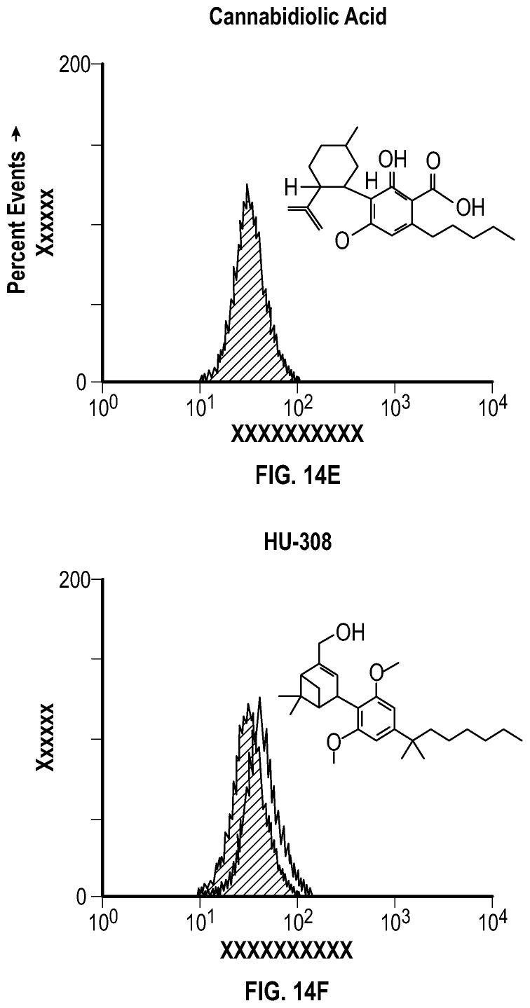

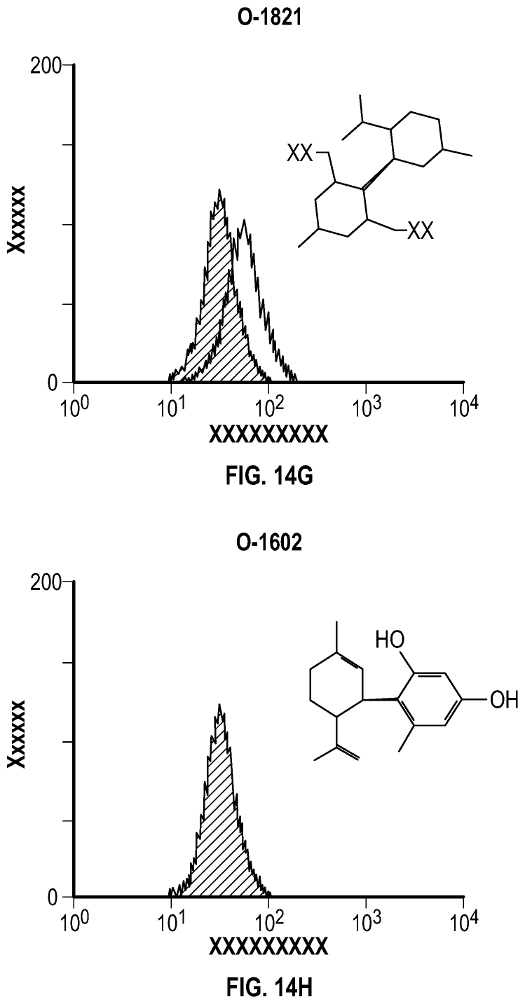

The present inventors demonstrate that chemical analogs of cannabidiol (CBD) elicit distinctly different biochemical responses in mammalian cells, indicating medicinal value of these analogs as replacements for, or as supplements to current statin cholesterol regulating medications. As generally shown in FIG. 14, CBD and some analogs of CBD elicit cellular responses similar to the cholesterol drugs Atorvastatin and Ezetimibe in SKNBE2 neuroblastoma cells, indicating CBD and some, but not all, CBD analogs cause a low cholesterol response.

### Example 2: Chemical Analogs of Cannabidiol (CBD) Exhibit Low Cellular Toxicity

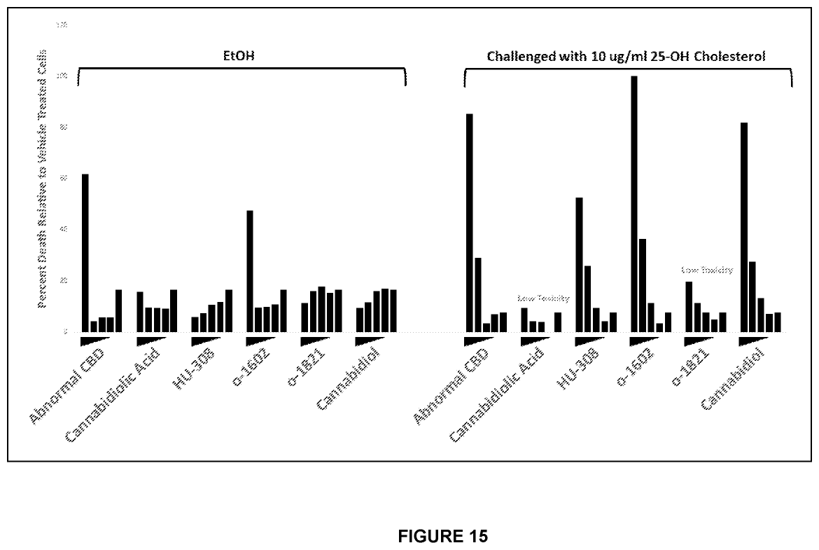

The present inventors demonstrate that CBD at high doses is toxic to SKNBE2 neuroblastoma cells that have been challenged with a high cholesterol environment, while, as shown generally in FIG. 15, select CBD chemical analogs show little to no toxicity.

### Example 3: Chemical Analogs of Cannabidiol (CBD) Cause Structural Changes in Cellular Endoplasmic Reticulum

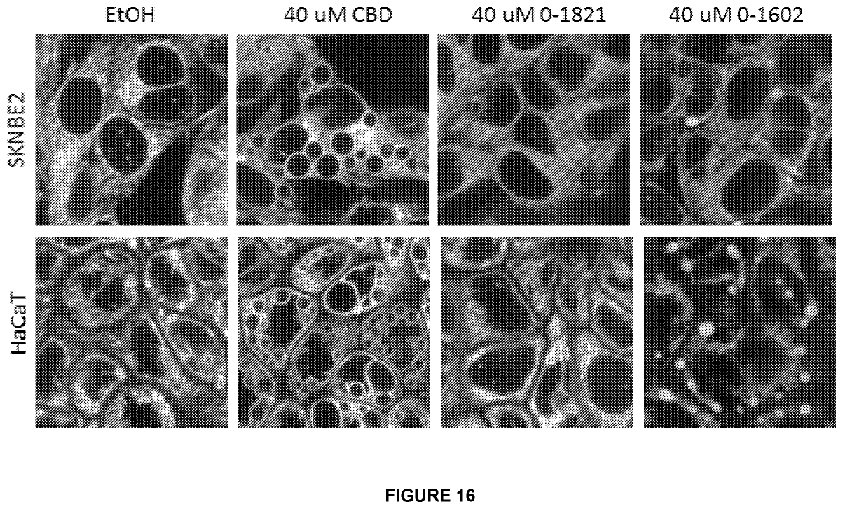

The present inventors further demonstrate that CBD dramatically alters the structure of the endoplasmic reticulum in SKNBE2 and HaCaT keratinocyte cells. As shown in FIG. 16, this effect is only observed in some (o-1602), but not all chemical analogs of CBD (o-1821, cannabidiolic acid, and abnormal CBD). As such, the present inventors demonstrate that, structural alteration of functional groups on the CBD molecule can influence toxicity, ER organelle structure and cholesterol sensing independently.

### Example 4: Chemical Analogs of Cannabidiol (CBD) Cause Increased Cholesterol Solubility and Accessibility to the Enzyme Cholesterol Oxidase

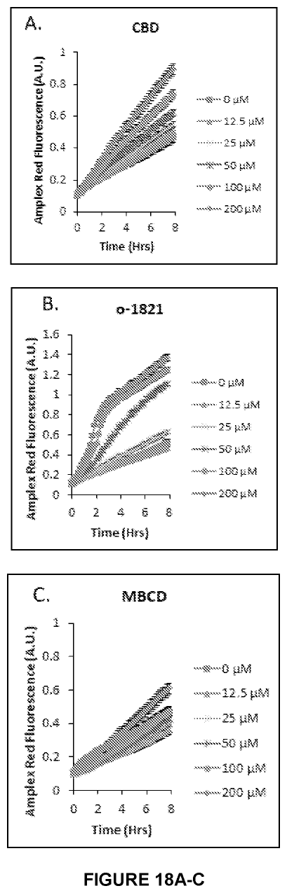

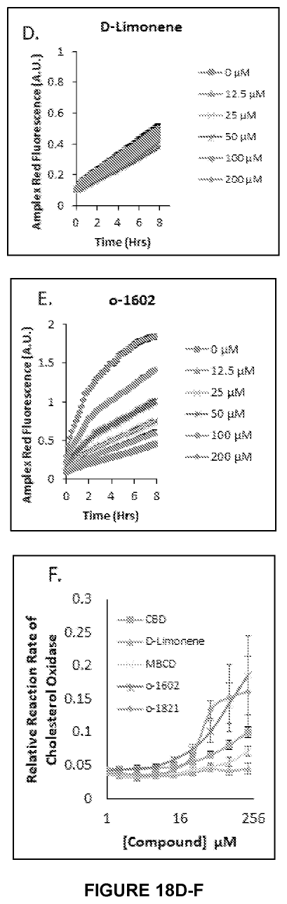

The present inventors demonstrate that treatment of cellular derived endoplasmic reticulum vesicles with CBD or analogs of CBD causes increased accessibility of cholesterol from within such membranes to the soluble enzyme cholesterol oxidase. As shown generally in FIG. 17, some, but not all, chemical analogs of CBD display such ability to alter cholesterol solubility. Specifically, as demonstrated in FIG. 17A-E, the present inventors demonstrated that enzymatic product formation of cholesterol oxidase was measured as accumulation of amplex red fluorescence over time using the amplex red cholesterol assay kit (ThermoFisher). Cholesterol was provided as a substrate trapped in endoplasmic derived membrane vesicles (ERVBs), which were extracted from living SKNBE2 neuroblastoma cells using subcellular fractionation by differential centrifugation. Cholesterol oxidase product formation was measured in the presence and absence of increasing doses of CBD, CBD analogs, or the cholesterol binding molecule methyl beta cyclodextrin (MBCD). As shown in FIG. 17F, the average reaction rate (0-8 hrs) of cholesterol oxidase in the presence of ERVBs was quantified as a function of concentration of CBD, CBD analogs, or MBCD.

### Example 5: Design and Execution of Multi-Omic Detection of Components in the CBD Drug Response

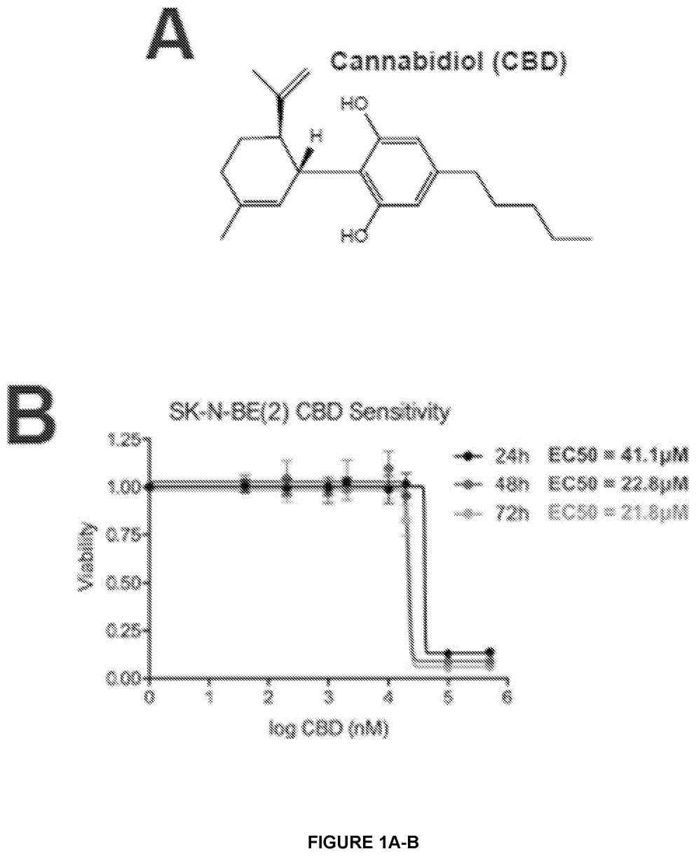

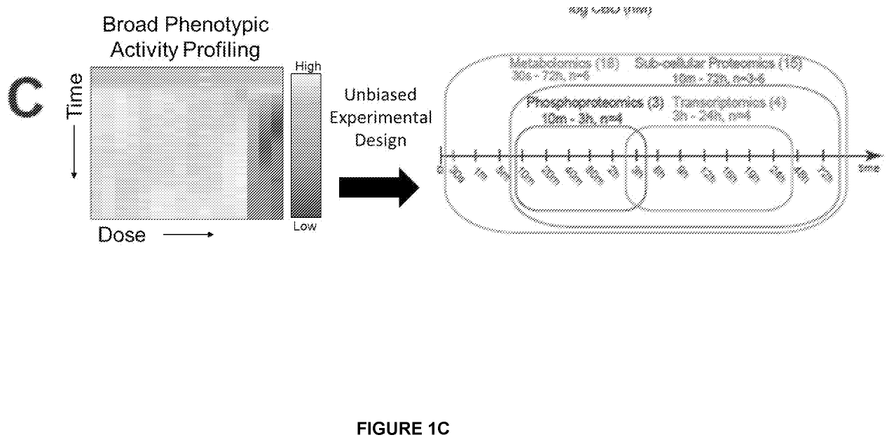

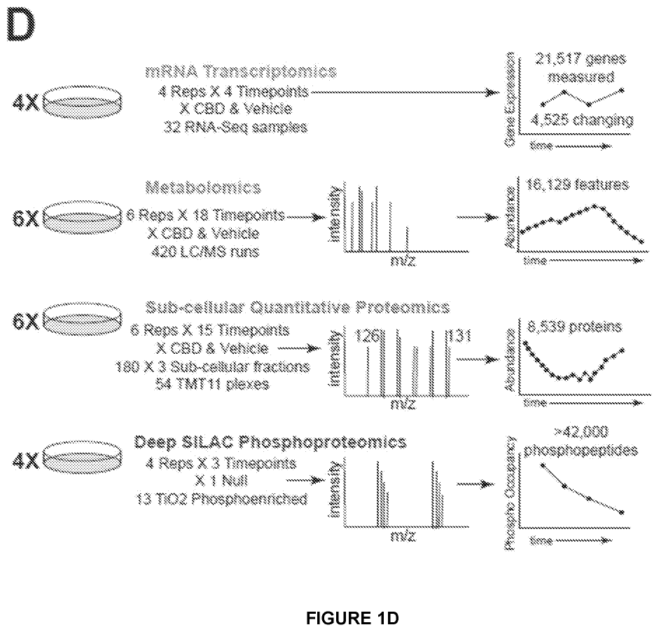

In a large-scale effort to demonstrate the potential of multiplexed-omics technologies to quickly inform key aspects of a drug's MoA at the cellular level, the present inventors developed a workflow that integrates mass spectrometry-based proteomics and metabolomics with next generation sequencing (NGS) based transcriptomics. The goal of this combined multi-omics approach is to lessen the bias in identifying components in a given drug's MoA via comprehensive quantification of drug specific perturbations to proteins, small molecules, and gene expression within cells. This technique was applied to gain insight into the biochemical MoA of CBD (structure depicted in FIG. 1A) in human cells in culture.

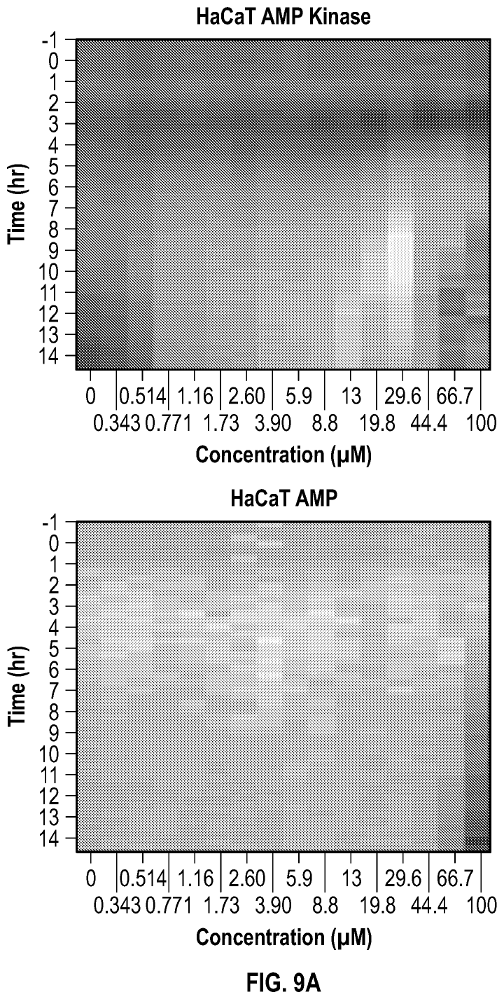

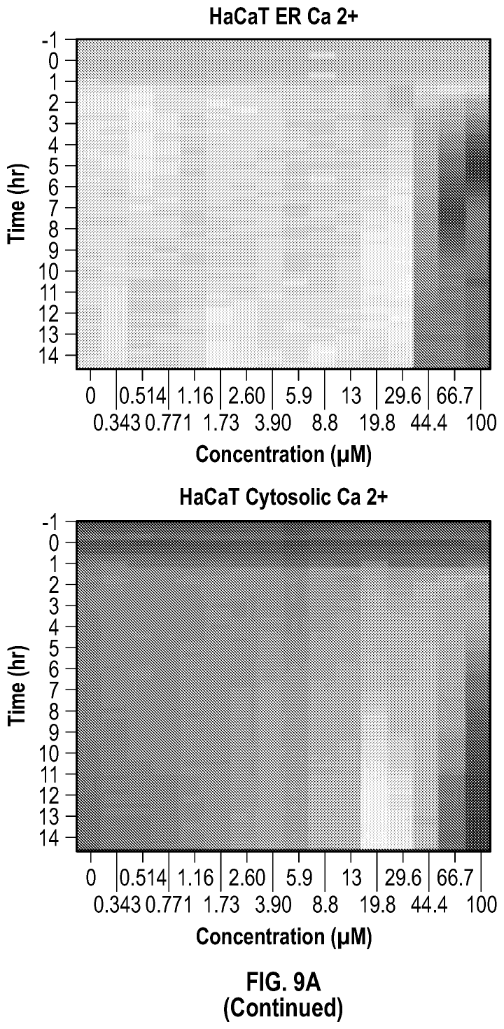

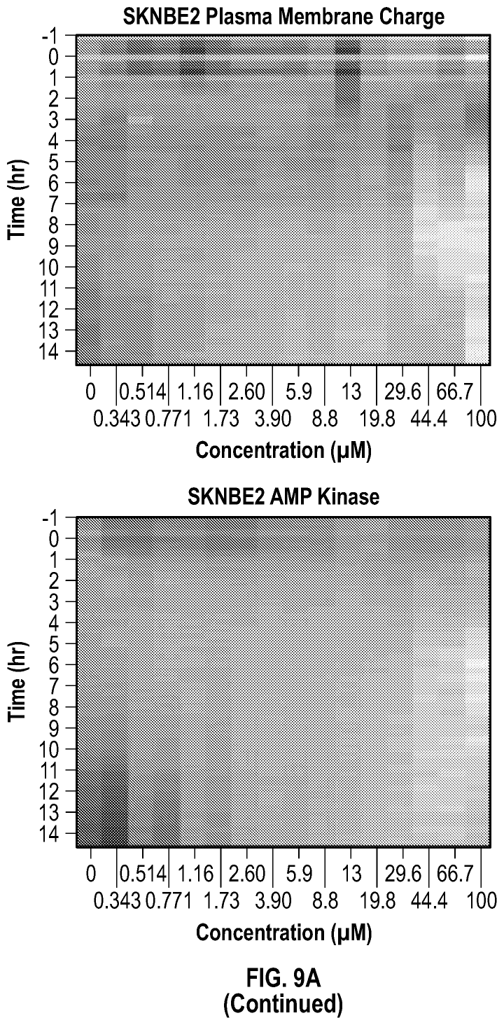

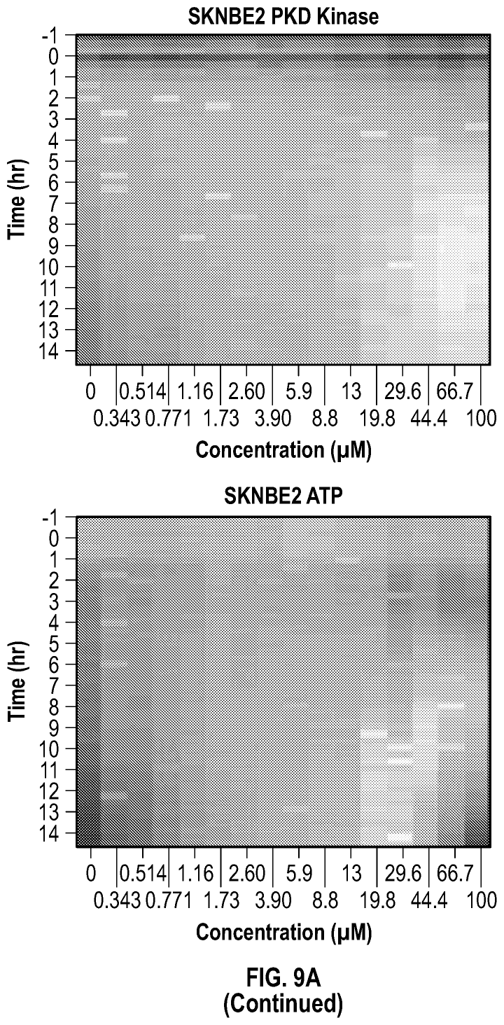

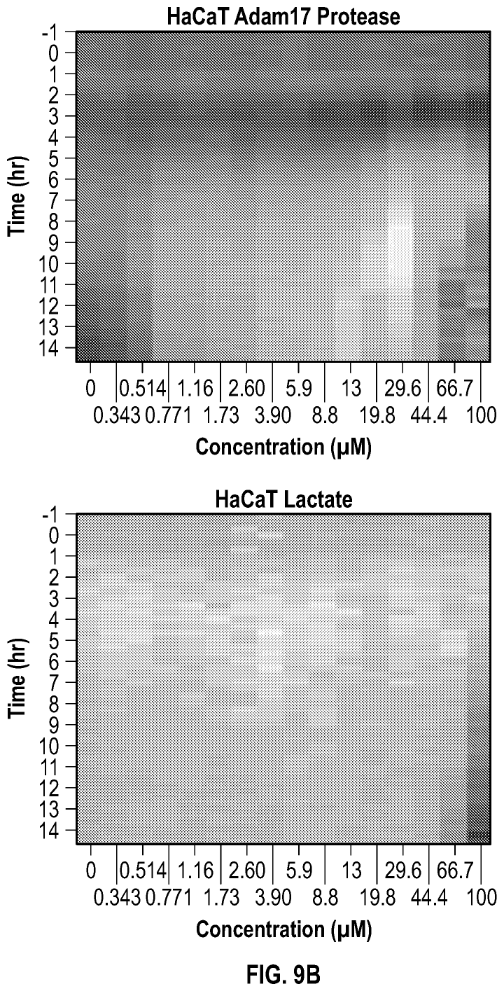

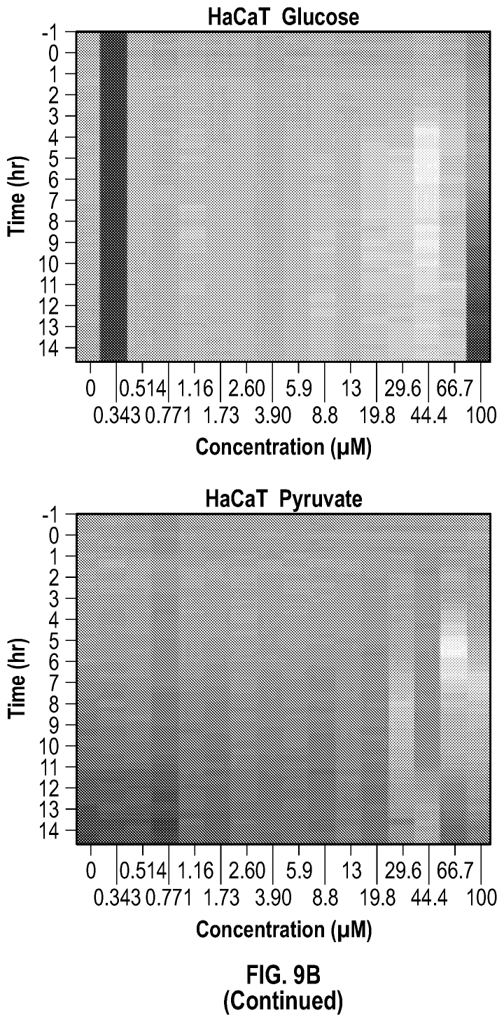

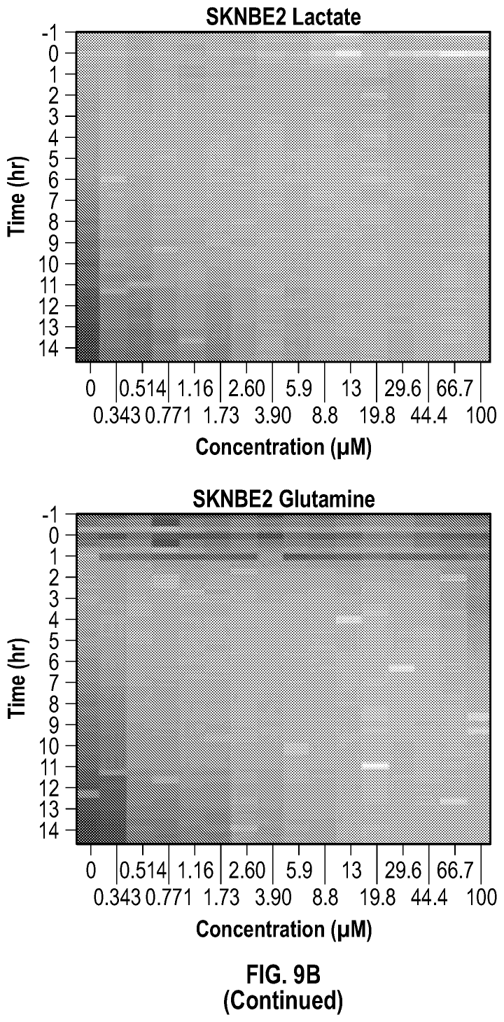

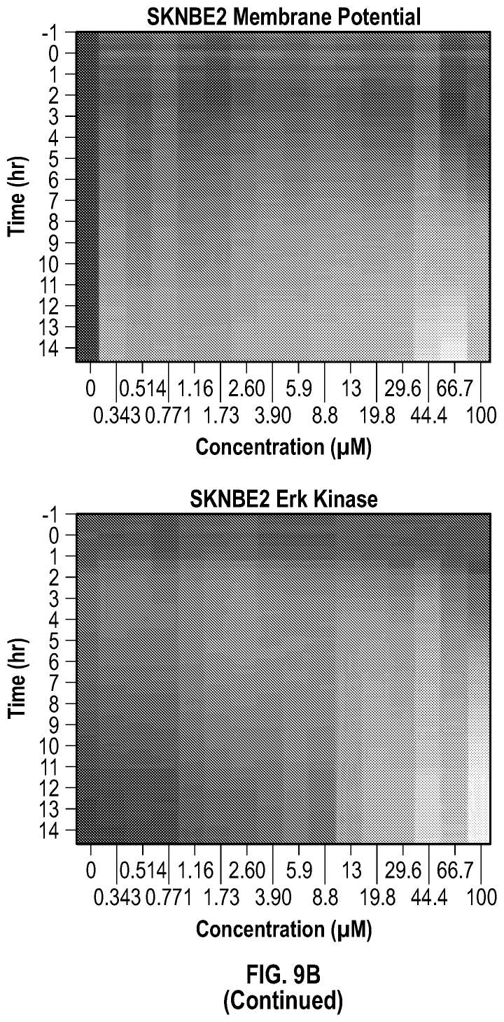

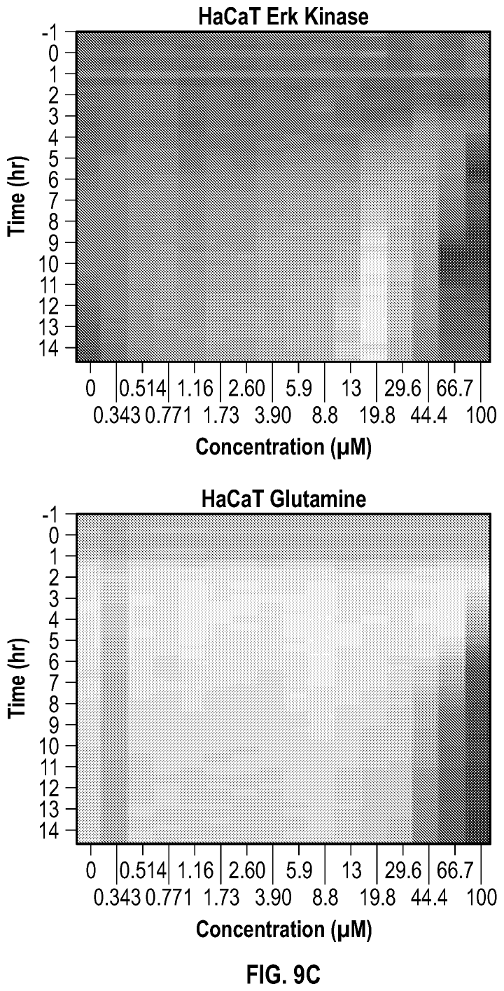

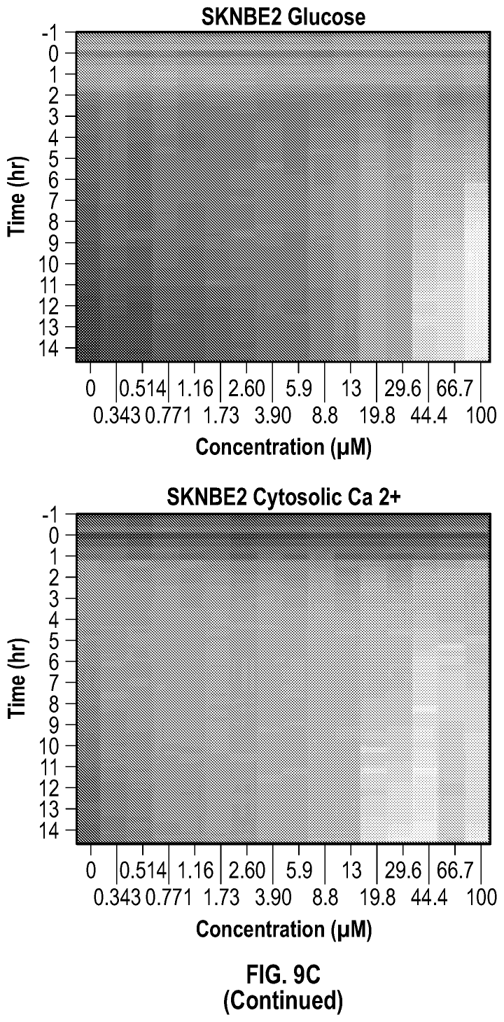

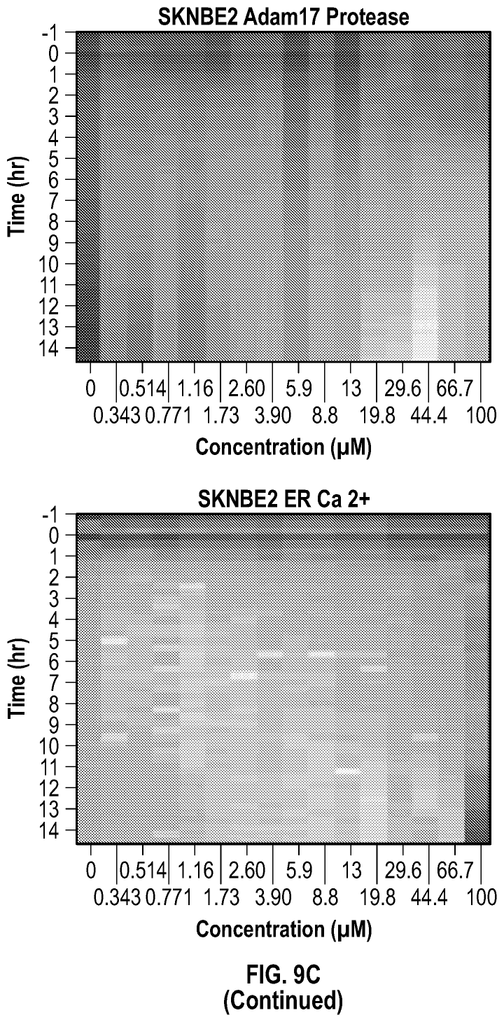

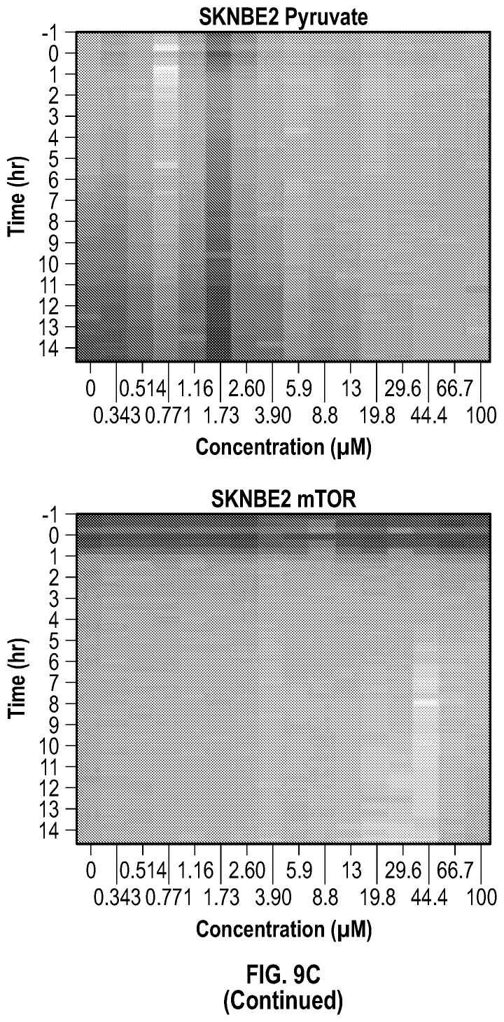

To efficiently design the multi-omics experiments, the present inventors exposed the neuroblastoma cell line SK-N-BE(2) to escalating doses of CBD at 24, 48, and 72 hours and determined the EC50 of toxicity of CBD to be greatest at 72 hours and at an approximate concentration of 20 μM (FIG. 1 B). To maximize the efficient use of the inventor's multi-omic resources, a large dose and time survey of the CBD response was conducted in a panel of transgenic SK-N-BE(2) and HaCaT keratinocyte cell lines, each expressing a genetically encoded förster resonance energy transfer (FRET) biosensor gene capable of reporting the activity of a cellular effector molecule/activity including, but not limited to: AMP kinase activity, Erk kinase activity, cytosolic ATP, glucose, lactate, pyruvate & glutamine, Ca²⁺ in the ER & Cytosol, mTor kinase activity & TACE protease activity. The full list of biosensors and their literature source, each being incorporated herein by reference, is displayed in Table 1. The biosensor activity profiles as a function of CBD dose and time (FIGS. 9A-C) were analyzed for the EC50 of each biosensor activity at each time (FIG. 10). The result of these efforts outlined that the most robust dose of CBD for use in multi-omic experiments is 20 μM and that the kinetic profile of the CBD response begins in hours and persists for days. Altogether, this information was used to design the multi-omic experiment detailed in FIGS. 1 C and D. In addition, the widespread effects of CBD on the biosensor activities throughout the panel served as an early indication that CBD causes modulation of all biochemical pathways that were probed, suggesting that the CBD response may originate from a pleiotropic source.

### Example 6: Proteomic and Transcriptomic Detection of Biochemical Components and Processes in the CBD Drug Response

In the proteomics arm of the multi-omic workflow, proteins that undergo robust drug dependent changes in either abundance or subcellular localization are identified. To achieve this goal, the present inventors developed a novel subcellular fractionation method schematically displayed in FIG. 2A and described in detail in the material and methods example below. Briefly, cells are collected, washed, and then lysed using mild detergent, exposed to two rounds of phase separation by centrifugation to isolate insoluble from soluble lysate components. This fractionation method was implemented over more traditional methods using Dounce homogenization because of the improved ability to efficiently process large numbers of samples in a high throughput manner. Similar methods of partitioning complex lysates using phase separation have been used in other studies and are commonly referred to as “differential solubility fractionation.” Peptides derived from each fraction from cells exposed to either vehicle or 20 μM CBD were analyzed for each time point and the sum of total events across all time points for each fraction was determined for both detected events (FIG. 2B, right chart) and CBD responsive events (FIG. 2B, left chart). The frequency of CBD responsive events detected in each fraction as a function of time indicates that each fraction contains proteins responsive to CBD treatment, as well as a relatively slow biochemical mechanism (hours to days) in the overall CBD drug response (FIG. 2C) that was consistent with CBD kinetics observed in the inventor's biosensor profiling experiments (FIG. 10). The relative CBD dependent effects on filtered protein identifications (FDR<1%) are tabulated for each fraction and time-point in FIG. 2D.

In order to compare and contrast proteomic derived mechanistic components of the CBD response to that of transcriptomic components, the present inventors additionally performed RNAseq analysis of SK-N-BE(2) cells exposed to vehicle or 20 μM CBD at the time-points indicated in FIG. 1C. 5,643 differentially expressed genes were identified in the CBD response gene with a FDR<1%, which was further reduced to a list of 139 genes with an LFC>=1. Differentially expressed transcript identifications that overlap with differentially regulated protein identifications from our proteomics experiments were included in the table displayed in FIG. 2D. FIG. 2D additionally displays the enriched biological processes for CBD responsive mechanistic components from these experiments, which include Translation, ER stress response, Metal Ion Response, and Cholesterol Biosynthesis. While most of these annotations are supported by either the transcriptome or the proteome, dysregulation of cholesterol metabolism is supported by both. Furthermore, 42 of the 150 proteins displayed in FIG. 2D have known roles in cholesterol import (i.e. APO-E and LDLR), cholesterol biosynthesis (i.e. SQLE and HMG-CR), or phospholipid catabolism (i.e. ENPP2). As a result of the agreement between proteomic and transcriptomic data, we focused our study thereafter on determining the role of cholesterol homeostasis in the CBD drug response.

### Example 7: Metabolomics Reveals CBD Dependent Inhibition of Cholesterol Biosynthesis

Proteomic and transcriptomic analyses revealed a widespread and concerted CBD-induced perturbation of cholesterol biosynthesis, trafficking and sensing. These findings raise the question of whether CBD treatment leads to alterations in lipid and cholesterol metabolism (the latter pathway depicted in FIG. 3A). The present inventors used mass spectrometry-based lipidomics, a subset of metabolomics, to quantify the effect of CBD on the fluxes of key lipids and sterols. Vehicle and CBD exposed cells were fed ¹³C labeled glucose for 24 hours and harvested using methanol extraction and mass spectrometry analysis. Cholesterol biosynthesis was analyzed by determining the level of incorporation of ¹³C into biosynthetic intermediates. The total and isotopically-labeled levels of virtually all cholesterol precursors were found to accumulate in CBD exposed cells, while labeled and total cholesterol itself was found to decrease in CBD exposed cells (FIGS. 3B, 11A). The effect of CBD on total cellular cholesterol was confirmed using a fluorogenic assay for cholesterol in an orthogonal experiment (FIG. 11B). The transcriptional activation of HMG-CR (FIG. 2D) combined with the increase in abundance of labelled cholesterol precursors (FIG. 3B) argues that the SREPB/SCAP cholesterol sensors in the ER have activated the cholesterol biosynthesis pathway in response to CBD.

To further investigate the role of SREBP/SCAP signaling in the CBD response, the present inventors used western blot analysis to probe whether SREBP2 proteolytic processing is affected by CBD. As shown in FIG. 3C by the increase in SREBP2 cleavage products (designated I for Intermediate and M for Mature), CBD exposure to cells causes dose dependent activation of SREBP2 cleavage. This activation is significant but is lower in magnitude than that of the pharmacological activators of SREBP2 cleavage, U18666A or Atorvastatin. The present inventors performed this assay with cells grown in LDL containing FBS (elevated cholesterol) and LDL depleted FBS (cholesterol starved). Surprisingly, CBD dose-dependent SREBP2 cleavage occurred both in cholesterol starved conditions and in elevated cholesterol conditions (FIG. 3C), suggesting that CBD can trigger transcriptional activation of cholesterol biosynthesis through SREBP processing even in cholesterol starved conditions. These data provide multiple lines of independent evidence that CBD transcriptionally promotes cholesterol biosynthetic genes and their activity, which is well known to be triggered by low amounts of cholesterol in the ER. However, the inventors also detect evidence that although cholesterol precursors are accumulating in a CBD dependent manner, cholesterol itself appears to experience a net decrease in de novo synthesis. This is evident in the overall decrease in newly synthesized ¹³C labeled cholesterol in CBD treated cells (FIG. 3B) and implies an apparent blockage in the final step of cholesterol biosynthesis, which is catalyzed by either DHCR24 or DHCR7 in the ER.

### Example 8: Lipidomics/Metabolomics Reveals CBD Dependent Activation of Cholesterol Storage

Elevated cholesterol storage via esterification may explain our observation of decreased cholesterol biosynthesis, which was observed in the metabolic flux experiments described above (FIG. 3B). To investigate the effect of CBD on cholesterol storage, the present inventors performed follow up experiments to determine the total cellular amounts of small molecule markers of cholesterol storage as a function of CBD exposure. The inventors detected accumulation of multiple species of cholesteryl esters with diverse chain length and degree of unsaturation in the acyl chain (FIG. 4A). Despite significantly higher levels of cholesteryl esters in CBD-treated cells, ¹³C labeled cholesteryl esters were not detected. The CBD-dependent increases in total unlabeled amounts of cholesteryl esters that we were able to detect suggest that cholesterol storage via cholesterol esterification is activated in the CBD response.

Since this esterification requires acyl CoA reactants, the inventors surveyed our dataset for evidence of fatty acid utilization. It was found that CBD treated cells display reduced free fatty acid levels (FIG. 4B), as well as accumulation of free phospholipid head-groups (FIG. 4C), including phospho-ethanolamine, a product of sphingosine catabolism via the enzyme S1P lyase. These data suggest that both free and S1P lyase generated fatty acids are likely utilized for cholesterol esterification.

The present inventors additionally surveyed the cellular abundance of all detectable species of phosphatidylcholine (PC) and phosphatidylethanolamine (PE) in cell extracts and found a variety of phospholipids that display reduced abundance in CBD treated vs vehicle treated cells (FIG. 4D), which is consistent with previous studies showing catabolic breakdown of phospholipids upstream of S1P lyase activity. Thus, the present inventors found multiple lines of evidence consistent with activation of cholesterol esterification in the CBD response. To further support the claim that CBD elicits cholesterol storage, the present inventors used live cell confocal microscopy of SK-N-BE(2) cells stained with both fluorescent cholesterol (22-NBD-Cholesterol) and the lipid droplet dye nile red (FIG. 4E) to quantify the number and size of lipid droplets. CBD increases the average abundance of lipid droplets (FIG. 4F) but does not affect lipid droplet size (FIG. 4G). Altogether, these combined experiments highlight that the CBD response in cells includes activation of cholesterol esterification and storage.

### Example 8: The Combination of CBD and Cholesterol Storage/Transport Inhibitors Induces Cholesterol Dependent Apoptosis in Multiple Cell Types

In order to shed light on the phenotypic implications of CBD disruption of cholesterol homeostasis, the present inventors sought to identify a critical phenotypic effect of CBD that is likely to result from altered cholesterol homeostasis. Earlier experiments described above indicated that high doses (>40 μM) of CBD can induce cell death after 24 hours (FIG. 1B). As a result, the inventors asked whether this cell death was related to cholesterol homeostasis.

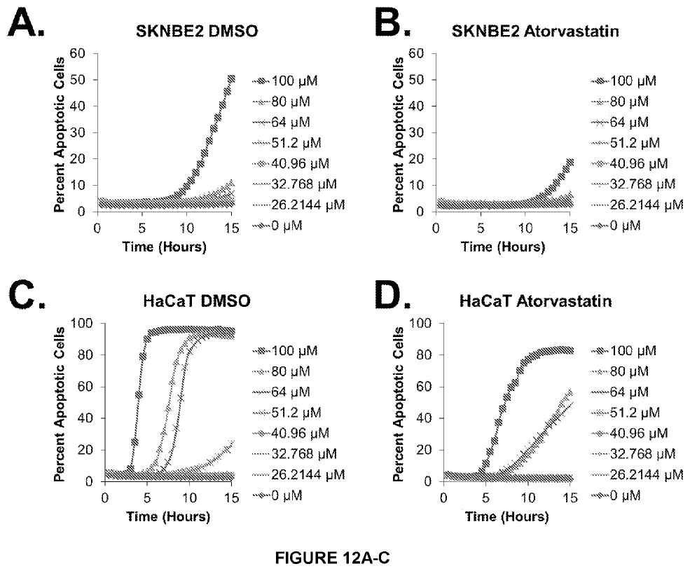

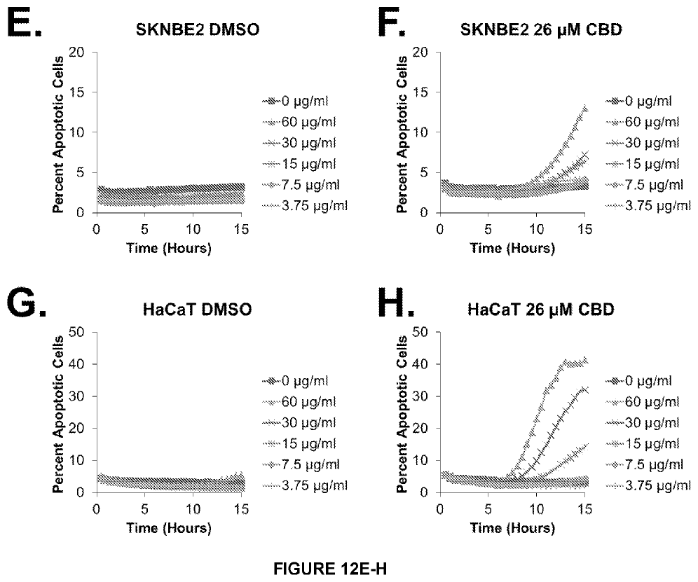

To accurately measure cellular apoptosis over time, the present inventors performed live cell microscopy experiments with cells loaded with both DNA and caspase 3/7 dyes, which enables continuous and precise quantification of apoptosis at the single cell level. Populations of SK-N-BE(2) cells exposed to increasing concentrations of CBD in the presence or absence of the HMG-CR inhibitor atorvastatin were analyzed for their percentage of apoptotic cells. The toxic effect of high CBD doses at 15 hours was found to be dramatically reduced by simultaneous exposure of CBD and atorvastatin, compared to CBD exposure alone (FIGS. 5A, 12A-B). This ability of inhibition of cholesterol biosynthesis to protect cells from CBD induced apoptosis was far more pronounced in the human keratinocyte cell line HaCaT, which was analyzed in the same manner as SK-N-BE(2) cells (FIGS. 5B, 12C-D). These data suggest that the cholesterol status of cells can greatly influence whether CBD causes apoptosis in both cell lines tested.

The inventors further tested this hypothesis by repeating our cellular apoptosis analyses in SK-N-BE(2) cells exposed to increasing concentrations of extracellular 25-hydroxy cholesterol in the presence of vehicle or constant CBD exposure. 25-hydroxy cholesterol is used here over cholesterol exposure because of its improved solubility, which circumvents the need for complex sterol delivery proteins. Similar to cholesterol, 25-hydroxy cholesterol has been shown to traffic efficiently to the ER and inhibit cholesterol biosynthesis via the SREBP/SCAP signaling axis. CBD exposed cells show a clear dose dependent sensitivity to 25-hydroxy cholesterol induced apoptosis (FIGS. 5C, 12E-F), which was more dramatically observed in HaCaT cells (FIGS. 5D, 12G-H). These data are consistent with our atorvastatin experiments, as both experiments suggest that CBD causes apoptosis in conditions where cells have high cholesterol/hydroxycholesterol.

In order to gain further insight into the sensitivity of CBD treated cells to 25-hydroxy cholesterol, the present inventors repeated the apoptosis assay in SK-N-BE(2) and HEK293T cells with combinatorial 25-hydroxy cholesterol/CBD stimulation using sublethal doses of 25-hydroxy cholesterol (15 μg/ml) and CBD (20 μM) in the presence or absence of either the chemical inhibitor of cholesterol transport (U18666A, 10 μM) or cholesterol storage (VULM 1457, 5 μM). Inhibition of cholesterol transport or cholesterol storage can each restore the apoptotic response of cells to combinatorial 25-hydroxy cholesterol/CBD treatment (FIGS. 5E, 12I-L), indicating that cholesterol transport and cholesterol storage are prosurvival in the CBD response.

One possible explanation for why CBD sensitizes cells to inhibitors of cholesterol trafficking and storage is that CBD increases the flux of cholesterol from the plasma membrane to the lysosome-ER transport pathway. When this flux is activated and hydroxy cholesterol is supplied in excess, the inability to efficiently store cholesterol may cause subcellular accumulation of cholesterol/hydroxycholesterol in organelles that normally maintain low cholesterol levels. To test this hypothesis, the inventor's repeated the live cell confocal microscopy experiments with SK-N-BE(2) cells stained with fluorescent cholesterol (NBD-cholesterol) and the lysosome dye lysotracker. Fluorescent cholesterol accumulates in labeled lysosomes after 24 hours only in the presence of the NPC1 inhibitor U18666A and CBD stimulation (FIG. 5F). This evidence demonstrates that CBD does indeed increase the flux of cholesterol through the lysosome and supports the hypothesis that CBD activates the intracellular transport of cholesterol. This data agrees with the present inventor's previous observations of CBD induced cholesterol esterification and storage (FIGS. 2 D, 3 A, 3 E-F), which occurs in the ER and lipid droplets, respectively. Altogether, these data outline a model where CBD triggers the plasma membrane to lipid droplet transport of cholesterol, which passes through the lysosome in route to the ER.

### Example 9: Membrane Incorporation of CBD Affects Cholesterol Orientation and Lateral Diffusion

In a metabolomics survey of SK-N-BE(2) cells exposed to CBD, the present inventors were able to determine that CBD is concentrated in insoluble components of the. As a result, the inventors designed and executed a subcellular fractionation method to isolate nuclear membrane, plasma membrane, and ER membrane fractions. The present inventors found that CBD is concentrated mostly at the plasma membrane at 24 hrs, although a detectable amount of CBD incorporates into ER and nuclear membranes (FIG. 6A).

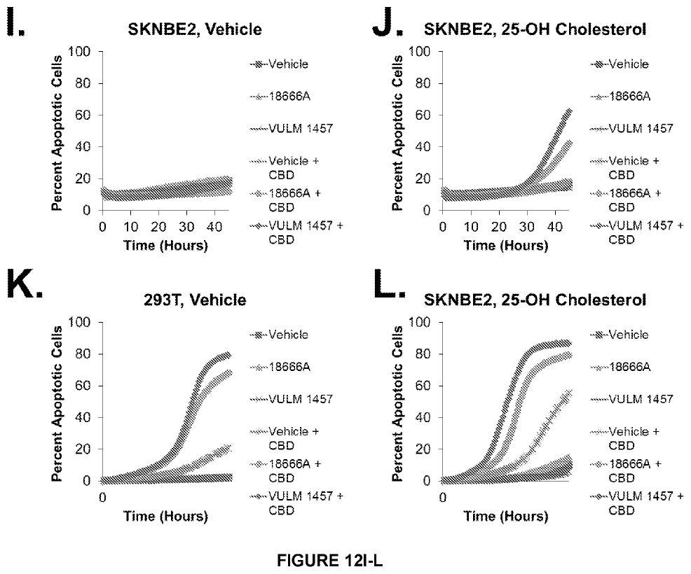

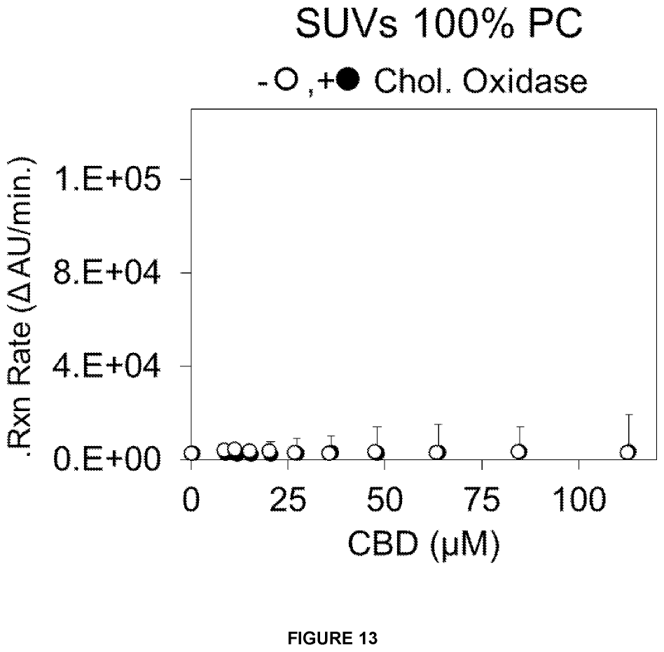

To further understand why CBD can induce cholesterol transport and storage, the present inventors tested the hypothesis that CBD might affect cholesterol directly. Using synthetic small unilamellular vesicles (SUVs) containing purified phosphatidyl choline (PC) and cholesterol, the inventors measured the relative ability of cholesterol to be converted to 5-cholesten-3-one by the enzyme cholesterol oxidase using a commercially available fluorogenic assay. Titration of CBD to cholesterol containing SUVs yields an increase in the reaction rate of cholesterol oxidase (FIG. 6B), but the same effect is not observed when this assay is repeated with SUVs that contain no cholesterol (FIG. 13). The same effect is also not observed when this experiment is repeated with cholesterol complexed with methyl beta cyclodextrin (MBCD) in solution (FIG. 6C) or with free soluble 25 hydroxy cholesterol (FIG. 6D). These data suggest that cholesterol is inside the membrane environment for CBD to affect the rate of enzymatic activity of cholesterol oxidase, implying that CBD increases the accessibility, or orientation, of cholesterol to cholesterol oxidase.

To validate that the same effect of CBD on cholesterol orientation can be observed in membranes with complex composition, the present inventors used a subcellular fractionation protocol to isolate ER membranes, which were found to be intact vesicles visualized using confocal microscopy and ER-tracker red staining (FIG. 5E). Using these vesicles as a cholesterol source yields identical results to SUVs in the cholesterol oxidase reaction rate experiments when exposed to increasing doses of CBD (FIG. 6F). Together, these data provide compelling evidence that CBD incorporates into membrane environments and alters cholesterol orientation such that the hydroxyl moiety of cholesterol is more solvent accessible.

CBD's ability to affect cholesterol orientation in both synthetic and cell derived ER membranes implies that CBD may contribute to increased lipid order. The ability of cholesterol oxidase assays to reveal alterations in lipid order has been previously reported in studies noting that cholesterol oxidase can preferentially target caveolar domains, which are ordered lipid domains. Another hallmark of lipid order is a decrease in lateral diffusion of lipids. As a result, the present inventors aimed to measure CBD's ability to perturb the lateral diffusion of fluorescently labelled cholesterol (NBD-cholesterol) in synthetic membranes. The present inventors deposited PC SUVs containing 20% moles of cholesterol and 2% moles NBD-cholesterol on glass-bottom multi-well imaging plates, followed by ultra-sonification to construct fluorescently labelled membrane monolayers. Using these monolayers and confocal microscopy, the inventor's performed fluorescence recovery after photobleaching (FRAP) experiments to measure the kinetics of recovery of fluorescent cholesterol. CBD significantly reduced FRAP kinetics (FIGS. 6G, H), suggesting that CBD slows the lateral diffusion of fluorescent cholesterol. This effect of CBD on lateral diffusion could be rescued with simultaneous treatment with the lipid disordering agent DHA (FIGS. 6G, I). There are two proposed models for how DHA affects cholesterol in membranes. In one model, DHA incorporates into highly disordered domains and excludes cholesterol, thus increasing partitioning of lipid ordered and disordered domains. The other proposed model is that DHA enters cholesterol rich domains and disrupts cholesterol packing. Both of these opposing models are supported by NMR experiments, but the latter model is favored by the observation that polyunsaturated fatty acids partition into detergent resistant membranes, implying that DHA associates with cholesterol rich domains. Consistent with this latter model, FRAP data suggests that DHA increases disorder within cholesterol containing domains resulting in an increase in the lateral diffusion of fluorescent cholesterol. As a result, the inventors concluded that the opposing effect of DHA and CBD on lateral fluorescent cholesterol diffusion supports CBD incorporation into membranes increases lipid order.

### Example 10: CBD Treatment Cancels the Apoptotic Effect of DHA

FRAP experiments reveal that DHA and CBD have opposing effects on the lateral diffusion of fluorescently labelled cholesterol in synthetic membranes. Although this implies that CBD increases lipid order and that DHA decreases lipid order, it remains unclear how relevant these biophysical effects of CBD and DHA are to cellular physiology. As a result, the present inventors sought to determine whether CBD and DHA have opposing effects on cells. To this end, the present inventors repeated the live cell apoptosis assays in cells exposed to increasing doses of DHA to determine how DHA affects cell death. The present inventors found that relatively low doses of DHA induce apoptosis in both HEK293T and SK-N-BE(2) cells (FIGS. 7A, B), which is consistent with previous studies. This DHA induced apoptosis is cholesterol dependent, as simultaneous treatment of DHA and the cholesterol sequestering agent MBCD can delay apoptosis in HEK293T cells and completely cancel apoptosis in SK-N-BE(2) cells (FIGS. 7 C, D). Since high doses of CBD also induce apoptosis (FIGS. 5A, B), we investigated whether low doses of CBD could counteract the apoptotic induction of DHA. To our surprise, CBD treatment can completely cancel the apoptotic effects of DHA in both HEK293T and SK-N-BE(2) cells (FIGS. 7 C, D). These data not only support the claim that CBD and DHA have opposing mechanisms of action to elicit cellular apoptosis, but also support the claim that these competing effects rely on the cholesterol content of cellular membranes.

### Example 11: Materials and Methods

### Cell Culture, Drug Treatments, and Harvesting.

SKNBE2 cells (ATCC CRL-2271) and HaCaT cells (Cell Line Service GmbH, Germany) were cultured in DMEM growth media (ThermoFisher, 11965118) supplemented with 10% fetal bovine serum, 2 mM L-Glutamine (ThermoFisher, 35050079), 50 μg/ml streptomycin and 50 U/ml Penicillin (ThermoFisher, 15070063). In proteomics and RNAseq experiments cells 2.5×10⁶ cells were seeded in 10 cm cell culture grade dishes. In metabolomics experiments cells 0.5×10⁶ cells were seeded in each well of a 12 well cell culture grade dishes. For all experiments, cells were seeded 36 hrs prior to drug treatments. Stock solutions of 100 mM CBD and 10 mg/ml 25-OH Cholesterol (Cayman Chemicals, 11097) were made using ethanol as a solvent. Atorvastatin (Cayman chemicals 10493) 20 mM stock solutions were made in DMSO. In proteomics and RNAseq experiments cells were harvested by washing plates with 10 ml room temperature phosphate buffered saline (PBS) three times, followed by rapid freezing with liquid nitrogen. For metabolomics experiments, cells were washed 2 times with 1 mL tris buffered saline (TBS), followed by addition of 1 ml ice cold ethanol. All plates were stored at −80° C. prior to subsequent processing.

### FRET Biosensor Profiling of CBD Response.

Stable transgenic biosensor expressing cell lines were made in HaCaT and SK-N-BE(2) cells. Briefly, biosensor gene containing plasmids were obtained through the addgene plasmid depository, and subcloned into our Bsr2 parent plasmid (sequence available upon request). Each biosensor Bsr2 plasmid was co-transfected with a PB recombinase expressing vector (mPB) via polymer based transfection using polyethyleneimine (PEI) (Polysciences, 25 kD Linear). Each stable transgenic cell line was selected for 7 days using 10 μg/ml Blasticidin S. FRET biosensor profiling was conducted in multiplexed parallel live cell experiments using 384 well imaging plates (Corning #3985) in an ImageXpress MicroXL high throughput microscope. Filters used for FRET measurements were the following: FRET excitation 438/24-25, dichroic 520LP, emission 542/27-25 (Semrock MOLE-0189); CFP excitation 438/24-25, dichroic 458LP, emission 483/32-25 (Semrock CFP-2432B-NTE-Zero). Time lapse microscopy images were collected, and FRET Ratio calculations for each site in each well at each time were performed as the mean value from pixels above threshold of background and flatfield corrected images, where each pixel value represented the FRET channel intensity divided by the CFP channel intensity. This method is described in more detail by Chapnick et al. Calculation and data visualization was performed in MATLAB using custom scripts that are available upon request.

### Transcriptomics Workflow:

Sequencing was performed on seven consecutive lanes. Median read counts per lane were ˜49,000 with a CV of ˜7%. Starting with 228 fastq files, each lane set was concatenated per condition. Using Trimmomtic-0.36, base pairs were trimmed if they dropped below a PHRED score of 15 within a sliding window of 4 bp. Remaining reads shorter than 35 base pairs were removed. Illumina adapter sequences were also clipped using Trimmomatic. Fastqc was used to verify data integrity. Read alignment was accomplished using Tophat-2.0.13 and Cufflinks/Cuffdiff 2.2.1 was used for FPKM quantitation and differential expression analysis per time point.

### Proteomics Bioinformatics Workflow:

Protein quantification for time-series was performed with a Tandem Mass Tag (TMT) isobarically labeled 11-plex multiplexing scheme. The 15-point time series for each cellular fraction was split into three series, with every series containing 5 treatment and matched control time point pairs, with 0 sec, 40 min, 3 hr, 12 hr, and 24 hr time points in Series A; 10 min, 80 min, 6 hr, 15 hr, and 48 hr in Series B; and 20 min, 2 hr, 9 hr, 18 hr, and 72 hr time points in Series C. This separation was performed so that a protein could be missing from one and/or two series due to stochastic effects in data-dependent acquisition and the overall trend could still be inferred, though with reduced resolution. The 11th label in each series was devoted to a global mix reference channel, which would be present in all series for a given cellular fraction. The global mix is a cell-fraction specific mixture that contains an equal portion from each time point sample. This channel was the denominator in the intermediate ratiometric measurement for differential expression for both drug-treated samples and time-matched controls. This mixture channel was constructed so that every measurable protein observed at any time point has a non-zero denominator when ratios are taken. When the differential expression is compared between the drug-treated labeled samples and matched control samples and expressed as a log₂ ratio, the global mix reference channel cancels out.

The differential expression of each individual protein was determined using Bayesian methods for isobarically labeled proteomics data. Briefly, all observed peptides are mapped to a list of observed protein ID's via Isoform Resolver. The TMT 11-plex reporter ion spectrum peaks for each peptide contributes to the inference of the differential expression of a protein and reporter ion label. In this case, each reporter ion label represents a particular measured time point. The label-to-label normalization is handled via a hierarchical model, which calculates the bias inherent with each specific label by pooling differential expression estimates from all proteins, changing and unchanging. The hierarchical model are solved computationally with a Markov Chain Monte Carlo (MCMC) method, running chains in parallel for faster results. The MCMC returns a Gaussian posterior probability distribution of logₑ differential expression for each protein for each label. The model initially fits ratiometric differential expression for every treatment and matched control relative to a global mix channel, and the reported drug-induced differential expression is the difference (in logₑ space) between the treated sample and the matched control sample. Five MCMC chains were run independently for at least 500k steps, and convergences for each run were verified via Gelman-Rubin convergence variable <1.05.

The differential expression was calculated independently for all biological replicates so protein-level variance from separate replicates could be examined and quantified in the posterior distributions obtained from MCMC. For reporting a single differential expression for a protein and label, the Bayesian updating procedure is used to produce a single posterior distribution, from which a mean point estimate and 95% credible interval are calculated. In some specific instances, labels represent technical rather than biological replicates. In cases of technical replicates, the point estimate values were averaged and the credible intervals extents were treated as errors and added in quadrature. With this procedure, technical replicates contribute a single probability distribution to any further Bayesian updating.

For every cellular fraction and time point, then, there are between 3 and 6 biological replicates, and the number of replicates represented in the drug treated samples and the matched control samples are not necessarily the same. The effect size (Cohen's d) was calculated between the posterior probability distributions of the drug treated and matched control samples as a standardized measure to determine if there was a drug effect. Statistical power analysis was performed to show that, with significance criteria α=0.05 and the statistical power requirement (1−β)=0.8, the appropriate effect size threshold should be d>(1.50, 1.75, 2.25, 3.38) for proteins observed within 6, 5, 4, or 3 replicates, respectively. A protein was selected for further consideration if it showed differential expression greater than this threshold for any given time point.

Bioconductor Edge version 2.8.0 was used for time course differential analysis. Many proteins were not present for all replicates and/or plexes, so Edge was run sequentially to generate p-values for each case. For instance, in the soluble fraction, there were 273 proteins that were only present in two replicates. These were run through Edge separately from the other 1957 proteins that were observed in three replicates. The resulting time series p-values were combined into a list and FDR corrected using Benjamini-Hochberg multiple hypothesis correction.

### Proteomics Network Analysis

Of the significantly changing proteins, correlation networks were generated for each sub-cellular fraction. Networks were created from the ethanol (vehicle) treated samples, as well as for the CBD treated samples. Network edge values were assigned using Spearman correlation coefficients between all proteins (vertices) for a given replicate. For each pair of proteins, 2*N edge values were generated, where N is the number of available replicate measurements for that protein. An independent t-test was used between basal replicate edge values and treatment edge values to evaluate what edges were significantly changed due to CBD treatment. Edges with −log₁₀(p-value)>2 (p<1%) were retained. Python graph-tool package was used generate a stochastic block-model representation of the resulting network, which clusters nodes based on network connectivity similarity.

### Subcellular Fractionation in Proteomics

For each sample, a 10 cm Petri Dish containing 10⁶ SKNBE2 cells was harvested and washed three times with 10 ml of 20° C. PBS. All PBS was removed by aspiration and plates were frozen using liquid nitrogen and stored at −80° C. overnight. Each plate was thawed on ice and 400 μl Tween20 Buffer (1×PBS, 0.1% Tween20, 5 mM EDTA, 30 mM NaF, 1 mM NaVo4, 100 μM Leupeptin, 2.5 μM Pepstatin A) and scraped thoroughly using a standard cell scraper. The resulting lysate was homogenized with a 200 μl pipette and transferred to 1.7 mL Eppendorf tube on ice. Lysate tubes were incubated for 30 min at 4° C. rotating end-over-end. After rotation, tubes were centrifuged for 10 min at 4° C. (16,100 rcf). All supernatant was transferred into new labeled 1.7 mL Eppendorf. This tube contains insoluble buoyant plasma membrane and cytosol. The leftover pellet is the ‘Insoluble #1’ fraction and is enriched in nuclei. 404, of 1 M NaOAc was added to the supernatants, which immediately were exposed to centrifugation for 10 min at 4° C. (16,100 rcf). All supernatant was transferred into new labeled 1.7 mL Eppendorf. This is the ‘Soluble’ fraction. The pellet was resuspended in 400 μl 20° C. SDS buffer. This is ‘Insoluble #2’ fraction. All fraction containing tubes were filled completely with −20° C. Acetone and stored overnight in −20° C. Each tube was exposed to centrifugation for 10 min at 4° C. (16,100 rcf) and supernatants were aspirated and discarded, while pellets were allowed pellets to air dry 10 min 20° C. The pellets then proceeded to the FASP procedure.

### Quantitative Subcellular Proteomics

Sample preparation. Precipitated and dried subcellular protein extracts were solubilized with 4% (w/v) sodium dodecyl sulfate (SDS), 10 mM Tris(2-carboxyethyl)phosphine (TCEP), 40 mM chloroacetamide with 100 mM Tris base pH 8.5. SDS lysates were boiled at 95° C. for 10 minutes and then 10 cycles in a Bioruptor Pico (Diagenode) of 30 seconds on and 30 second off per cycle, or until protein pellets were completely dissolved. Samples were then cleared at 21,130×g for 10 minutes at 20° C., then digested into tryptic peptides using the filter-aided sample preparation (FASP) method (Wisniewski (2016) Analytical Chemistry 88, 5438). Briefly, SDS lysate samples were diluted 10-fold with 8M Urea, 0.1M Tris pH8.5 and loaded onto an Amicon Ultra 0.5 mL 30 kD NMWL cutoff (Millipore) ultrafiltration device. Samples were washed in the filters three time with 8M Urea, 0.1M Tris pH8.5, and again three times with 0.1M Tris pH8.5. Endoproteinase Lys-C(Wako) was added and incubated 2 hours rocking at room temperature, followed by trypsin (Pierce) which was incubated overnight rocking at room temperature. Tryptic peptides were eluted via centrifugation for 10 minutes at 10,000×g, and desalted using an Oasis HLB cartridge (Waters) according to the manufacture instructions.

TMT labeling. Cleaned up and dried tryptic peptides were suspended in 30 uL 0.1M triethylammonium bicarbonate (TEAB), peptide concentration was determined by absorbance at 280 nm (nanodrop 2000, Thermo Scientific) and peptide concentrations were adjusted to 1.0 ug/uL with 0.1M TEAB. Ten micrograms of each sample was labeled with a TMT 10plex kit (Thermo Scientific 90406). Labeling was performed for 1 hour at ambient, and the unreacted label was quenched with hydroxylamine for 15 minutes. The 10plexed samples were then combined and cleaned up using an Oasis HLB cartridge (Waters).

High pH C18 fractionation of TMT labeled peptides. Dried 10plexed samples were then suspended in 20 uL 3% (v/v) acetonitrile (ACN) and 0.1% (v/v) trifluoroacetic acid (TFA) and loaded onto a custom fabricated reverse phase C18 column (0.5×200 mm C18, 1.8 um 120 Å Uchrom (nanoLCMS Solutions) maintained at 25° C. and running 15 uL/min with buffer A, 10 mM ammonium formate, pH 10 and buffer B, 10 mM ammonium formate, pH10 in 80% (v/v) ACN with a Waters M-class UPLC (Waters). Peptides were separated by gradient elution from 3% B to 50% B in 25 minutes, then from 50% B to 100% B in 5 minutes. Fractions were collected in seven rounds of concatenation for 30 sec per fraction, then combined for a final of six high pH C18 fractions. Samples were dried and stored at −80° C. until ready for LC/MS analyses.

Liquid Chromatography/Mass spectrometry analysis. Samples were suspended in 3% (v/v) acetonitrile, 0.1% (v/v) trifluoroacetic acid and direct injected onto a 1.7 um, 130 Å C18, 75 um×250 mm M-class column (Waters), with a Waters M-class UPLC or a nanoLC1000 (Thermo Scientific). Tryptic peptides were gradient eluted at 300 nL/minute, from 3% acetonitrile to 20% acetonitrile in 100 minutes into an Orbitrap Fusion mass spectrometers (Thermo Scientific). Precursor mass spectrums (MS1) were acquired at 120,000 resolution from 380-1500 m/z with an automated gain control (AGC) target of 2.0E5 and a maximum injection time of 50 ms. Dynamics exclusion was set for 15 seconds with a mass tolerance of +/−10 ppm. Quadrupole isolation for MS2 scans was 1.6 Da sequencing the most intense ions using Top Speed for a 3 second cycle time. All MS2 sequencing was performed using collision induced dissociation (CID) at 35% collision energy and scanned in the linear ion trap. An AGC target of 1.0E4 and 35 second maximum injection time was used. Selected-precursor selections of MS2 scans was used to isolate the five most intense MS2 fragment ions per scan to fragment at 65% collision energy using higher energy collision dissociation (HCD) with liberated TMT reporter ions scanned in the orbitrap at 60,000 resolution. An AGC target of 1.0E5 and 240 second maximum injection time was used for all MS3 scans. All raw files were converted to mzML files and searched against the Uniprot Human database downloaded Apr. 1, 2015 using Mascot v2.5 with cysteine carbamidomethylation as a fixed modification, methionine oxidation, and protein N-terminal acetylation were searched as variable modifications. Peptide mass tolerance was 20 ppm for MS1 and 0.5 mDa for MS2. All peptides were thresholded at a 1% false discovery rate (FDR).

### Phosphoproteomics

Sample preparation and phosphoenrichment. SKNBE2 cells were cultured in SILAC media either with Lys8 and Arg10 (Heavy) or Lys0 and Arg0 (Light). Two biological replicates of near confluent Heavy cells and two replicates of near confluent Light cells were treated with 20 uM CBD for 10 minutes (4 replicates), 1 hour (4 replicates) and 3 hours (4 replicates) for phosphoproteomics analyses. Cells were harvested in 4% (w/v) SDS, 100 mM Tris, pH 8.5 and boiled at 95° C. for 5 minutes. Samples were reduced with 10 mM TCEP and alkylated with 50 mM chloroacetamide, then digested using the FASP protocol, with the following modifications: an Amicon Ultra 0.5 mL 10 kD NMWL cutoff (Millipore) ultrafiltration device was used rather than a 30 kD NMWL cutoff. Tryptic peptides were cleaned a Water HLB Oasis cartridge (Waters) and eluted with 65% (v/v) ACN, 1% TFA. Glutamic acid was added to 140 mM and TiO (Titanshere, GL Sciences) was added at a ratio of 10 mg TiO:1 mg tryptic peptides and incubated for 15 minutes at ambient. The phosphopeptide-bound TiO beads were washed with 65% (v/v) ACN, 0.5% TFA and again with 65% (v/v) ACN, 0.1% TFA, then transferred to a 200 uL C8 Stage Tip (Thermo Scientific). Phosphopeptides were eluted with 65% (v/v) ACN, 1% (v/v) ammonium hydroxide and lyophilized dry.

High pH C18 fractionation of phosphoenriched peptides. Phosphoenriched samples were then suspended in 20 uL 3% (v/v) acetonitrile (ACN) and 0.1% (v/v) trifluoroacetic acid (TFA) and loaded onto a custom fabricated reverse phase C18 column (0.5×200 mm C18, 1.8 um 120 Å Uchrom (nanoLCMS Solutions) maintained at 25° C. and running 15 uL/min with buffer A, 10 mM ammonium formate, pH 10 and buffer B, 10 mM ammonium formate, pH10 in 80% (v/v) ACN with a Waters M-class UPLC (Waters). Peptides were separated by gradient elution from 3% B to 50% B in 25 minutes, then from 50% B to 100% B in 5 minutes. Fractions were collected in seven rounds of concatenation for 30 sec per fraction for a final of twelve high pH C18 fractions. Samples were dried and stored at −80° C. until analysis.

Liquid Chromatography/Mass spectrometry analysis of phosphopeptide fractions. Samples were suspended in 3% (v/v) acetonitrile, 0.1% (v/v) trifluoroacetic acid and direct injected onto a 1.7 um, 130 Å C18, 75 um×250 mm M-class column (Waters), with a Waters M-class UPLC. Tryptic peptides were gradient eluted at 300 nL/minute, from 3% acetonitrile to 20% acetonitrile in 100 minutes into an Orbitrap Fusion mass spectrometer (Thermo Scientific). Precursor mass spectrums (MS1) were acquired at 120,000 resolution from 380-1500 m/z with an AGC target of 2.0E5 and a maximum injection time of 50 ms. Dynamics exclusion was set for 20 seconds with a mass tolerance of +/−10 ppm. Isolation for MS2 scans was 1.6 Da using the quadrupole, and the most intense ions were sequenced using Top Speed for a 3 second cycle time. All MS2 sequencing was performed using higher energy collision dissociation (HCD) at 35% collision energy and scanned in the linear ion trap. An AGC target of 1.0E4 and 35 second maximum injection time was used. Rawfiles were searched against the Uniprot human database using Maxquant with cysteine carbamidomethylation as a fixed modification. Methionine oxidation, protein N-terminal acetylation, and phosphorylation of serine, threonine and tyrosine were searched as variable modifications. All peptides and proteins were thresholded at a 1% false discovery rate (FDR).

### Bulk Metabolomics Sample Preparation

Cultured cells were harvested, washed with PBS, flash frozen, and stored at −80 C. until analysis. Prior to LC-MS analysis, samples were placed on ice and re-suspended with methanol:acetonitrile:water (5:3:2, v/v/v) at a concentration of 2 million cells per ml. Suspensions were vortexed continuously for 30 min at 4° C. Insoluble material was removed by centrifugation at 10,000 g for 10 min at 4° C. and supernatants were isolated for metabolomics analysis by UHPLC-MS. This method was used for cholesterol precursors and free headgroups.

UHPLC-MS analysis for Bulk Metabolomics

The analytical platform employs a Vanquish UHPLC system (Thermo Fisher Scientific, San Jose, Calif., USA) coupled online to a Q Exactive mass spectrometer (Thermo Fisher Scientific, San Jose, Calif., USA). Samples were resolved over a Kinetex C18 column, 2.1×150 mm, 1.7 μm particle size (Phenomenex, Torrance, Calif., USA) equipped with a guard column (SecurityGuard™ Ultracartridge—UHPLC C18 for 2.1 mm ID Columns—AJO-8782—Phenomenex, Torrance, Calif., USA) (A) of water and 0.1% formic acid and a mobile phase (B) of acetonitrile and 0.1% formic acid for positive ion polarity mode, and an aqueous phase (A) of water:acetonitrile (95:5) with 1 mM ammonium acetate and a mobile phase (B) of acetonitrile:water (95:5) with 1 mM ammonium acetate for negative ion polarity mode. Samples were eluted from the column using either an isocratic elution of 5% B flowed at 250 μl/min and 25° C. or a gradient from 5% to 95% B over 1 minute, followed by an isocratic hold at 95% B for 2 minutes, flowed at 400 μl/min and 30° C. The Q Exactive mass spectrometer (Thermo Fisher Scientific, San Jose, Calif., USA) was operated independently in positive or negative ion mode, scanning in Full MS mode (2 μscans) from 60 to 900 m/z at 70,000 resolution, with 4 kV spray voltage, 15 sheath gas, 5 auxiliary gas. Calibration was performed prior to analysis using the Pierce™ Positive and Negative Ion Calibration Solutions (Thermo Fisher Scientific). Acquired data was then converted from .raw to .mzXML file format using Mass Matrix (Cleveland, Ohio, USA). Metabolite assignments, isotopologue distributions, and correction for expected natural abundances of deuterium, ¹³C, and ¹⁵N isotopes were performed using MAVEN (Princeton, N.J., USA). Graphs, heat maps and statistical analyses (either T-Test or ANOVA), metabolic pathway analysis, PLS-DA and hierarchical clustering was performed using the MetaboAnalyst package.

### Lipidomics Sample Preparation

Extraction of cholesterol, precursors, free fatty acids, cholesteryl esters, and phospho lipids were performed in the following manner. SK-N-BE(2) cells in 10 cm dishes were washed with 10 mL PBS twice and then cells were scraped and pelleted at 400 rcf for 2 minutes. Cell pellets were resuspended in 100% methanol at 4° C. and sonicate at 70% power in 10 pulses, 5 seconds on/5 seconds off. The resulting lysate was rotated for 60 minutes at room temperature, followed by centrifugation for 20 min at 4° C. (16,100 rcf). Subcellular fractionation of organelles from intact SK-N-BE(2) cells was done in the following manner to assess subcellular CBD distribution. Cells in 10 cm culture dishes were harvest by washing twice with 10 mL PBS at room temperature, followed by trypsinization using a cell culture grade Trypsin/EDTA solution (ThermoFisher). Trypsinized cells were quenched by addition of 2 mL 10% FBS containing DMEM and cells were pelleted by centrifugation for 2 min at 4° C. (200 rcf). Cell pellets were wash one time with 10 mL PBS, and resuspended in 1 mL Tween20 Buffer (1×PBS, 0.05% Tween20, 5 mM EDTA). This lysate was subjected to mechanical disruption using a 1 mL glass Dounce homogenizer, 10 full passes at 4° C. Nuclei was pelleted from homogenate by centrifugation for 5 min at 4° C. (2,000 rcf). Supernatant was separated and insoluble ER membranes were pelleted by centrifugation for 10 min at 4° C. (4,000 rcf). Supernatant was separated and insoluble plasma membranes were pelleted by centrifugation for 10 min at 4° C. (16,000 rcf). Extraction of all fractions was done in 100% methanol for 2 hours at room temperature and rotation end-over-end, followed by removal of insolubles by centrifugation for 20 min at 20° C. (16,100 rcf).

UHPLC-MS analysis for Lipidomics

Analytes were resolved over an ACQUITY HSS T3 column (2.1×150 mm, 1.8 μm particle size (Waters, Mass., USA) using an aqueous phase (A) of 25% acetonitrile and 5 mM ammonium acetate and a mobile phase (B) of 90% isopropanol, 10% acetonitrile and 5 mM ammonium acetate. The column was equilibrated at 30% B, and upon injection of 101 of extract, samples were eluted from the column using the solvent gradient: 0-9 min 30-100% B and 0.325 mL/min; hold at 100% B for 3 min at 0.3 mL/min, and then decrease to 30% over 0.5 min at 0.4 ml/min, followed by a re-equilibration hold at 30% B for 2.5 minutes at 0.4 ml/min. The Q Exactive mass spectrometer (Thermo Fisher) was operated in positive ion mode, scanning in Full MS mode (2 μscans) from 150 to 1500 m/z at 70,000 resolution, with 4 kV spray voltage, 45 shealth gas, 15 auxiliary gas. When required, dd-MS2 was performed at 17,500 resolution, AGC target=1e5, maximum IT=50 ms, and stepped NCE of 25, 35 for positive mode, and 20, 24, and 28 for negative mode. Calibration was performed prior to analysis using the Pierce™ Positive and Negative Ion Calibration Solutions (Thermo Fisher). Acquired data was then converted from .raw to .mzXML file format using Mass Matrix (Cleveland, Ohio, USA). Samples were analyzed in randomized order with a technical mixture injected incrementally to qualify instrument performance. This technical mixture was also injected three times per polarity mode and analyzed with the parameters above, except CID fragmentation was included for unknown compound identification. Metabolite assignments were made based on accurate intact mass (sub 5 ppm), isotope distributions, and relative retention times, and comparison to analytical standards in the SPLASH Lipidomix Mass Spec Standard (Avanti Polar Lipids) using MAVEN (Princeton, N.J., USA). Discovery mode analysis was performed with standard workflows using Compound Discoverer and Lipid Search 4.0 (Thermo Fisher Scientific, San Jose, Calif.).

### Western Blot Analysis of SREBP2 Proteolytic Processing.

SK-N-BE(2) cells were cultured as described above in the presence of either 10% FBS or 10% LDL-Depleted FBS (Kalen Biomedical, LLC) for 24 hours prior to addition of CBD at the indicated doses, U18666A (10 μM) or Atorvastatin (10 μM). Cells were harvested at 24 hours by trypsinization and counted using a hemocytometer to determine cell concentration. 4×10\hat{ }6 cells from each sample were lysed using 200 μL SDS lysis buffer (20 mM Tris pH 6.8, 4% SDS, 0.5% beta mercaptoethanol) and lysates were boiled for 5 minutes 95° C. and subsequently sonicated at 40% power 10 cycles 10 seconds on/off. 10 μL of lysate was loaded into each lane of a 12% SDS PAGE gel. Immunoblotting of protein transferred to nitrocellulose membrane was performed using anti-SREBP2 (Cayman Chemical #10007663).

### Confocal Microscopy of Lipid Droplets and Lysosomes.

SK-N-BE(2) cells were seeded into fibronectin coated glass bottom 96 well plates (Matriplate) at a cell density of 40,000 cells/well using low background imaging media (FluoroBrite DMEM with all supplements described, above). At the time of seeding, nile red (ThermoFisher) was added at a final concentration of 100 ng/mL and NBD-cholesterol (ThermoFisher) was added at a final concentration of 10 μg/mL. After 24 hrs, CBD or ethanol vehicle was added to a final concentration of 20 μM and incubated for an additional 24 hrs prior to imaging using a Nikon MR laser scanning confocal microscope for acquisition with the FITC and TRITC channels. Lipid droplets and their sizes were detected and quantified using the ImageJ Find Particles Function, and data was subsequently processed using Microsoft Excel. Error depicted represents the standard deviation calculated from three biological replicates. For experiments visualizing NBD-cholesterol and a lysosomal marker, an identical procedure was used with a substitution of the lysotracker Deep Red dye (ThermoFisher) which was used at a 1000× dilution. In experiments using U18666A, a final concentration of 10 μg/ml was used and was added simultaneously with CBD.

### Assaying Cell Viability and Apoptosis.

Cell viability for SK-N-BE(2) cells was conducted using a fluorometric cell viability assay using Resazurin (PromoKine) according to the manufacturer's instructions. Measurement of percent apoptotic cells was done in 384 well imaging plates (Corning #3985) seeded with 2,000 cells/well and stained with Hoescht 33258 (1 μg/mL) and CellEvent Caspase-3/7 Green Detection Reagent (ThermoFisher) at a dilution of 1000×. Dyes were added at the time of seeding, 18-24 hours prior to performing experiments. For experiments using atorvastatin, atorvastain was added 24 hrs prior to addition of CBD. For experiments involving 25-hydroxy cholesterol, U18666A, and VULM 1457, inhibitors were added simultaneously with CBD.

Experiments were performed using an ImageXpress MicroXL microscope and a 10× objective, where images were acquired for each well at the indicated time-points using DAPI and FITC filter sets. Using MATLAB, images were processed with custom written scripts (available upon request) that perform flatfield & background correction, identification of all cells (DAPI channel) using standard threshold above background techniques, and identification of apoptotic cells using a similar method in the FITC channel. Percent apoptotic cells was calculated from the sum of apoptotic cell pixels divided by the sum of all cell pixels for each field of view. Error displayed is the standard deviation from between 2 and 4 biological replicates.

### Fluorometric Cholesterol Oxidase Experiments and SUV Preparation.

SUVs were prepared by dissolving 10 mg L-a-Phosphatidylcholine (Sigma P3556) in 100 chloroform in a glass vial, followed by removal of solvent using vacuum distillation at room temperature for 1 hour. For conditions of cholesterol containing SUVs, 0.74 mg of cholesterol (Sigma C8667) was mixed with 10 mg L-a-Phosphatidylcholine prior to removal of chloroform solvent. Following solvent removal, 100 μL PBS was added and a microtip sonicator was inserted to perform sonication at 70% power, 10 pulses, 5 seconds on/off at room temperature. SUVs in suspension were brought to a volume of 1 mL with addition of PBS. The resulting SUVs in suspension were used at a dilution of 100× in subsequent cholesterol oxidase reactions. Cholesterol oxidase reactions were performed using reagents from the Amplex Red Cholesterol Assay Kit (ThermoFisher A12216), where each reaction was performed in 50 μL volumes using: 0.5 μL SUVs solution, 0.05 μL cholesterol oxidase solution, 0.05 μL HRP solution, 0.05 Amplex Red/DMSO made according to manufacturer's instructions, and the indicated CBD concentrations in PBS. Reaction volume was brought to 50 μL using PBS. In cases where SUVs were not used, either 1 μg/reaction 25-OH cholesterol or 5 μg/reaction MBCD:Cholesterol (1:2) was substituted for SUVs. MBCD:Cholesterol was prepared as described by Widenmaier, et al. Cholesterol oxidase reactions were performed in Corning 384 well optical imaging plates (#3985) in an ImageXpress MicroXL widefield fluorescence microscope using the TRITC filter sets, where 1 ms exposure time images were taken of each well 20 μm above the well bottom every 10 minutes for 5 hours at 37° C., using a 10× objective. Images were flatfield corrected and the sum of fluorescence intensity across all 540×540 pixels was calculated using custom MATLAB scripts that are available upon request. Product formation of Amplex red was found to be linear within 0-1 hours, and data between t=0 and t=1 hours was used to calculate the average rate of increase in TRITC fluorescence using microsoft Excel. Displayed error bars represent the standard deviation of three or more replicate reactions.

### FRAP Experiments Using Synthetic Membranes.

The formulation and techniques to create SUVs were repeated with addition of 108 μg of 22-NBD cholesterol to L-a-Phosphatidylcholine and cholesterol prior to the solvent removal step described for preparation of SUVs. A 1:5 dilution of NBD-cholesterol containing SUV suspension:PBS was added to each well of glass bottom 96 well plates (MatriPlate MGB096-1-2-LG-L) such that each well contains 100 μl of diluted SUV suspension. 96 well plates were exposed to centrifugation for 20 minutes at 2000 rcf using a swinging bucket rotor at room temperature. A microtip sonicator was inserted into each well to perform sonication at 20% power, 20 pulses, 2 seconds on/off at room temperature. The contents of each well was washed three times with 150 μl of PBS, and subsequent experiments were performed with 250 μl PBS containing ethanol vehicle, 20 μM CBD and/or 20 μM DHA. CBD and DHA were incubated in wells for 1 hr at room temperature prior to imaging and FRAP experiments using a Nikon MR microscope. Photobleaching was performed using Nikon Elements software with the following parameters: framerate 250 ms, 100% power 488 laser for photobleaching for 250 ms, and optical settings for FITC. Analysis was performed using ImageJ and Microsoft Excel. All trends were normalized by division of mean intensity within the photobleached region to a region of identical size remote from the photobleached region. Error bars indicate the standard error of the mean from three replicates.

### TABLES

TABLE 1
Biosensors used for profiling the EC50 Effects of CBD. The name
of each genetically encoded biosensor gene used to profile diverse
activities in cell lines is displayed along with its target analyte and
literature source.

| Targeted | Biosensor | |
|---|---|---|
| Analyte | Gene Name | Original Literature Source |
| mTOR Kinase | TORCAR | Zhou X, Clister T L, Lowry P R, Seldin |
| Activity | | M M, Wong G W, Zhang J (2015) |
| | | Dynamic Visualization of mTORC1 |
| | | Activity in Living Cells. Cell reports |
| AMP Kinase | AMPKAR | Tsou P, Zheng B, Hsu CH, Sasaki A T, |
| Activity | | Cantley L C (2011) A fluorescent |
| | | reporter of AMPK activity and cellular |
| | | energy stress. Cell metabolism |
| | | 13 (4): 476-486 |
| Cytosolic Ca²⁺ | D3-cpv | Ravier M A, Cheng-Xue R, Palmer |
| Abundance | | A E, Henquin J C, Gilon P (2010) |
| | | Subplasmalemmal Ca(2+) |
| | | measurements in mouse pancreatic |
| | | beta cells support the existence of an |
| | | amplifying effect of glucose on |
| | | insulin secretion. Diabetologia |
| | | 53 (9): 1947-1957 |
| Adam17 | TSEN | Chapnick D A, Bunker E, Liu X (2015) |
| Protease | | A biosensor for the activity of the |
| Activity | | ″sheddase″ TACE (ADAM17) reveals |
| | | novel and cell type-specific |
| | | mechanisms of TACE activation. |
| | | Science signaling 8 (365) |
| Lactate | Laconic | San Martin A, Ceballo S, Ruminot I, |
| Abundance | | Lerchundi R, Frommer W B, Barros |
| | | L F (2013) A genetically encoded |
| | | FRET lactate sensor and its use to |
| | | detect the Warburg effect in single |
| | | cancer cells. PloS one 8 (2) |
| ATP Abundance | ATEAM | Imamura H, Nhat K P, Togawa H, |
| | | Saito K, Iino R, Kato-Yamada Y, |
| | | Nagai T, Noji H (2009) Visualization |
| | | of ATP levels inside single living cells |
| | | with fluorescence resonance energy |
| | | transfer-based genetically encoded |
| | | indicators. Proceedings of the |
| | | National Academy of Sciences of |
| | | the United States of America |
| | | 106 (37): 15651-15656 |
| PKD Kinase | DKAR | Kunkel M T, Toker A, Tsien R Y, |
| Activity | | Newton A C (2007) Calcium-dependent |
| | | regulation of protein kinase D revealed |
| | | by a genetically encoded kinase activity |
| | | reporter. The Journal of biological |
| | | chemistry 282 (9): 6733-6742 |
| ER Ca²⁺ | D1ER | Palmer A E, Jin C, Reed J C, Tsien R Y |
| Abundance | | (2004) Bcl-2-mediated alterations in |
| | | endoplasmic reticulum Ca2+ analyzed |
| | | with an improved genetically encoded |
| | | fluorescent sensor. Proceedings of the |
| | | National Academy of Sciences of |
| | | the United States of America |
| | | 101 (50): 17404-17409 |
| Plasma | VSFP-CR | Lam A J, St-Pierre F, Gong Y, |
| Membrane | | Marshall J D, Cranfill P J, Baird M A, |
| Potential | | McKeown M R, Wiedenmann J, |
| | | Davidson M W, Schnitzer M J, |
| | | Tsien R Y, Lin M Z (2012) Improving |
| | | FRET dynamic range with bright |
| | | green and red fluorescent proteins. |
| | | Nature methods 9 (10): 1005-1012 |
| Glucose | FLIPglu- | Takanaga H, Frommer W B (2010) |
| Abundance | 30uDelta13V | Facilitative plasma membrane |
| | | transporters function during ER |
| | | transit. FASEB journal: official |
| | | publication of the Federation of |
| | | American Societies for Experimental |
| | | Biology 24 (8): 2849-2858 |
| Glutamine | FLIPQTV3.0 | Gruemvald K, Holland J T, |
| Abundance | 8m | Stromberg V, Ahmad A, |
| | | Watcharakichkom D, Okumoto |
| | | S (2012) Visualization of |
| | | glutamine transporter activities |
| | | in living cells using genetically |
| | | encoded glutamine sensors. |
| | | PloS one 7 (6) |
| Cytosolic ERK | EKAR-NES | Komatsu N, Aoki K, Yamada M, |
| Kinase Activity | | Yukinaga H, Fujita Y, Kamioka Y, |
| | | Matsuda M (2011) Development of |
| | | an optimized backbone of FRET |
| | | biosensors for kinases and GTPases. |
| | | Molecular biology of the cell |
| | | 22 (23): 4647-4656 |
| Plasma | MCS+ | Ma Y, Yamamoto Y, Nicovich P R, |
| Membrane | | Goyette J, Rossy J, Gooding J J, |
| Electrostatic | | Gaus K (2017) A FRET sensor |
| Potential | | enables quantitative measurements |
| (Charge) | | of membrane charges in live cells. |
| | | Nature biotechnology 35 (4): 363-370 |
| Pyruvate | Pyronic | San Martin A, Ceballo S, |
| Abundance | | Baeza-Lehnert F, Lerchundi R, |
| | | Valdebenito R, Contreras-Baeza Y, |
| | | Alegria K, Barros L F (2014) |
| | | Imaging mitochondrial flux in single |
| | | cells with a FRET sensor for pyruvate. |
| | | PloS one 9 (1) |

### REFERENCES

[1] Adams, C. M., Reitz, J., De Brabander, J. K., Feramisco, J. D., Li, L., Brown, M. S., and Goldstein, J. L. (2004). Cholesterol and 25-hydroxycholesterol inhibit activation of SREBPs by different mechanisms, both involving SCAP and Insigs. J. Biol. Chem. 279, 52772-52780.
[2] Aguilar, A., and Saba, J. D. (2012). Truth and consequences of sphingosine-1-phosphate lyase. Adv. Biol. Regul. 52, 17-30.
[3] Alharris, E., Singh, N. P., Nagarkatti, P. S., and Nagarkatti, M. (2019). Role of miRNA in the regulation of cannabidiol-mediated apoptosis in neuroblastoma cells. Oncotarget 10, 45-59.
[4] Appelqvist, H., Nilsson, C., Garner, B., Brown, A. J., Kågedal, K., and Öllinger, K. (2011). Attenuation of the lysosomal death pathway by lysosomal cholesterol accumulation. Am. J. Pathol. 178, 629-639.
[5] Appelqvist, H., Sandin, L., Björnström, K., Saftig, P., Garner, B., Öllinger, K., and Kågedal, K. (2012). Sensitivity to Lysosome-Dependent Cell Death Is Directly Regulated by Lysosomal Cholesterol Content. PLoS ONE 7.
[6] Aqul, A., Liu, B., Ramirez, C. M., Pieper, A. A., Estill, S. J., Burns, D. K., Liu, B., Repa, J. J., Turley, S. D., and Dietschy, J. M. (2011). Unesterified cholesterol accumulation in late endosomes/lysosomes causes neurodegeneration and is prevented by driving cholesterol export from this compartment. J. Neurosci. Off. J. Soc. Neurosci. 31, 9404-9413.
[7] Brown, M. S., and Goldstein, J. L. (1997). The SREBP pathway: regulation of cholesterol metabolism by proteolysis of a membrane-bound transcription factor. Cell 89, 331-340.
[8] Brown, M. S., and Goldstein, J. L. (2009). Cholesterol feedback: from Schoenheimer's bottle to Scap's MELADL. J. Lipid Res. 50 Suppl, S15-27.
[9] Chang, T.-Y., Li, B.-L., Chang, C. C. Y., and Urano, Y. (2009). Acyl-coenzyme A:cholesterol acyltransferases. Am. J. Physiol.—Endocrinol. Metab. 297, E1-E9.
[10] Chapnick, D. A., Bunker, E., and Liu, X. (2015). A biosensor for the activity of the “sheddase” TACE (ADAM17) reveals novel and cell type-specific mechanisms of TACE activation. Sci. Signal. 8, rs1.
[11] Cheng, D., Chang, C. C., Qu, X., and Chang, T. Y. (1995). Activation of acyl-coenzyme A:cholesterol acyltransferase by cholesterol or by oxysterol in a cell-free system. J. Biol. Chem. 270, 685-695.
[12] Commissioner, O. of the Press Announcements—FDA approves first drug comprised of an active ingredient derived from marijuana to treat rare, severe forms of epilepsy.
[13] Corroon, J., and Kight, R. (2018). Regulatory Status of Cannabidiol in the United States: A Perspective. Cannabis Cannabinoid Res. 3, 190-194.
[14] De Filippis, D., Esposito, G., Cirillo, C., Cipriano, M., De Winter, B. Y., Scuderi, C., Sarnelli, G., Cuomo, R., Steardo, L., De Man, J. G., et al. (2011). Cannabidiol reduces intestinal inflammation through the control of neuroimmune axis. PloS One 6, e28159.
[15] Drug Enforcement Administration, Department of Justice (2018). Schedules of Controlled Substances: Placement in Schedule V of Certain FDA-Approved Drugs Containing Cannabidiol; Corresponding Change to Permit Requirements. Final order. Fed. Regist. 83, 48950-48953.
[16] Esposito, G., Scuderi, C., Valenza, M., Togna, G. I., Latina, V., De Filippis, D., Cipriano, M., Carratú, M. R., Iuvone, T., and Steardo, L. (2011). Cannabidiol Reduces Aβ-Induced Neuroinflammation and Promotes Hippocampal Neurogenesis through PPARγ Involvement. PLoS ONE 6.
[17] Ferreri, C. (2005). Life—As a Matter of Fat: The Emerging Science of Lipidomics. By Ole G. Mouritsen. ChemBioChem 6, 1463-1464.
[18] Fraldi, A., Annunziata, F., Lombardi, A., Kaiser, H.-J., Medina, D. L., Spampanato, C., Fedele, A. O., Polishchuk, R., Sorrentino, N. C., Simons, K., et al. (2010). Lysosomal fusion and SNARE function are impaired by cholesterol accumulation in lysosomal storage disorders. EMBO J. 29, 3607-3620.
[19] Gaston, T. E., Bebin, E. M., Cutter, G. R., Liu, Y., Szaflarski, J. P., and UAB CBD Program (2017). Interactions between cannabidiol and commonly used antiepileptic drugs. Epilepsia 58, 1586-1592.
[20] Geng, L., Zhou, W., Liu, B., Wang, X., and Chen, B. (2018). DHA induces apoptosis of human malignant breast cancer tissues by the TLR-4/PPAR-α pathways. Oncol. Lett. 15, 2967-2977.
[21] Georgieva, R., Chachaty, C., Hazarosova, R., Tessier, C., Nuss, P., Momchilova, A., and Staneva, G. (2015). Docosahexaenoic acid promotes micron scale liquid-ordered domains. A comparison study of docosahexaenoic versus oleic acid containing phosphatidylcholine in raft-like mixtures. Biochim. Biophys. Acta BBA—Biomembr. 1848, 1424-1435.
[22] Giannini, E. G., Testa, R., and Savarino, V. (2005). Liver enzyme alteration: a guide for clinicians. CMAJ Can. Med. Assoc. J. 172, 367-379.
[23] Goswami, D., Gowrishankar, K., Bilgrami, S., Ghosh, S., Raghupathy, R., Chadda, R., Vishwakarma, R., Rao, M., and Mayor, S. (2008). Nanoclusters of GPI-anchored proteins are formed by cortical actin-driven activity. Cell 135, 1085-1097.
[24] Graham, J. M. (2015). Fractionation of Subcellular Organelles. Curr. Protoc. Cell Biol. 69, 3.1.1-22.
[25] Gruenwald, K., Holland, J. T., Stromberg, V., Ahmad, A., Watcharakichkorn, D., and Okumoto, S. (2012). Visualization of glutamine transporter activities in living cells using genetically encoded glutamine sensors. PloS One 7, e38591.
[26] Hancock, J. F. (2006). Lipid rafts: contentious only from simplistic standpoints. Nat. Rev. Mol. Cell Biol. 7, 456-462.
[27] Hande, K. (2019). Cannabidiol: The Need for More Information About Its Potential Benefits and Side Effects. Clin. J. Oncol. Nurs. 23, 131-134.
[28] Hao, M., Mukherjee, S., Sun, Y., and Maxfield, F. R. (2004). Effects of cholesterol depletion and increased lipid unsaturation on the properties of endocytic membranes. J. Biol. Chem. 279, 14171-14178.
[29] Hinz, B., and Ramer, R. (2018). Anti-tumour actions of cannabinoids. Br. J. Pharmacol.
[30] Holden, P., and Horton, W. A. (2009). Crude subcellular fractionation of cultured mammalian cell lines. BMC Res. Notes 2, 243.
[31] Hsu, J.-L., Leu, W.-J., Hsu, L.-C., Liu, S.-P., Zhong, N.-S., and Guh, J.-H. (2018). Para-Toluenesulfonamide Induces Anti-tumor Activity Through Akt-Dependent and -Independent mTOR/p70S6K Pathway: Roles of Lipid Raft and Cholesterol Contents. Front. Pharmacol. 9, 1223.
[32] Ibeas Bih, C., Chen, T., Nunn, A. V. W., Bazelot, M., Dallas, M., and Whalley, B. J. (2015). Molecular Targets of Cannabidiol in Neurological Disorders. Neurother. J. Am. Soc. Exp. Neurother. 12, 699-730.
[33] Imamura, H., Nhat, K. P. H., Togawa, H., Saito, K., Iino, R., Kato-Yamada, Y., Nagai, T., and Noji, H. (2009). Visualization of ATP levels inside single living cells with fluorescence resonance energy transfer-based genetically encoded indicators. Proc. Natl. Acad. Sci. U.S.A 106, 15651-15656.
[34] Kenyon, J., Liu, W., and Dalgleish, A. (2018). Report of Objective Clinical Responses of Cancer Patients to Pharmaceutical-grade Synthetic Cannabidiol. Anticancer Res. 38, 5831-5835.
[35] Kim, J. B., and Spiegelman, B. M. (1996). ADD1/SREBP1 promotes adipocyte differentiation and gene expression linked to fatty acid metabolism. Genes Dev. 10, 1096-1107.
[36] Kim, J. H., Singh, A., Del Poeta, M., Brown, D. A., and London, E. (2017). The effect of sterol structure upon clathrin-mediated and clathrin-independent endocytosis. J. Cell Sci. 130, 2682-2695.
[37] Komatsu, N., Aoki, K., Yamada, M., Yukinaga, H., Fujita, Y., Kamioka, Y., and Matsuda, M. (2011). Development of an optimized backbone of FRET biosensors for kinases and GTPases. Mol. Biol. Cell 22, 4647-4656.
[38] Kunkel, M. T., Toker, A., Tsien, R. Y., and Newton, A. C. (2007). Calcium-dependent regulation of protein kinase D revealed by a genetically encoded kinase activity reporter. J. Biol. Chem. 282, 6733-6742.
[39] Lam, A., St-Pierre, F., Gong, Y., Marshall, J. D., Cranfill, P. J., Baird, M. A., McKeown, M. R., Wiedenmann, J., Davidson, M. W., Schnitzer, M. J., et al. (2012). Improving FRET dynamic range with bright green and red fluorescent proteins. Nat. Methods 9, 1005-1012.
[40] Lange, Y., Ye, J., Rigney, M., and Steck, T. L. (1999). Regulation of endoplasmic reticulum cholesterol by plasma membrane cholesterol. J. Lipid Res. 40, 2264-2270.
[41] Lenne, P.-F., Wawrezinieck, L., Conchonaud, F., Wurtz, O., Boned, A., Guo, X.-J., Rigneault, H., He, H.-T., and Marguet, D. (2006). Dynamic molecular confinement in the plasma membrane by microdomains and the cytoskeleton meshwork. EMBO J. 25, 3245-3256.
[42] Levental, K. R., Lorent, J. H., Lin, X., Skinkle, A. D., Surma, M. A., Stockenbojer, E. A., Gorfe, A. A., and Levental, I. (2016). Polyunsaturated Lipids Regulate Membrane Domain Stability by Tuning Membrane Order. Biophys. J. 110, 1800-1810.
[43] Liao, G., Yao, Y., Liu, J., Yu, Z., Cheung, S., Xie, A., Liang, X., and Bi, X. (2007). Cholesterol accumulation is associated with lysosomal dysfunction and autophagic stress in Npc1−/− mouse brain. Am. J. Pathol. 171, 962-975.
[44] Lindblom, G., and Oradd, G. (2009). Lipid lateral diffusion and membrane heterogeneity. Biochim. Biophys. Acta BBA—Biomembr. 1788, 234-244.
[45] Lingwood, D., and Simons, K. (2010). Lipid rafts as a membrane-organizing principle. Science 327, 46-50.
[46] Liu, X., and Fagotto, F. (2011). A Method to Separate Nuclear, Cytosolic, and Membrane-Associated Signaling Molecules in Cultured Cells. Sci. Signal. 4, pl2-pl2.
[47] Luu, W., Hart-Smith, G., Sharpe, L. J., and Brown, A. J. (2015). The terminal enzymes of cholesterol synthesis, DHCR24 and DHCR7, interact physically and functionally. J. Lipid Res. 56, 888-897.
[48] Ma, Y., Yamamoto, Y., Nicovich, P. R., Goyette, J., Rossy, J., Gooding, J. J., and Gaus, K. (2017). A FRET sensor enables quantitative measurements of membrane charges in live cells. Nat. Biotechnol. 35, 363-370.
[49] Massi, P., Solinas, M., Cinquina, V., and Parolaro, D. (2013). Cannabidiol as potential anticancer drug. Br. J. Clin. Pharmacol. 75, 303-312.
[50] Matsuda, M., Korn, B. S., Hammer, R. E., Moon, Y.-A., Komuro, R., Horton, J. D., Goldstein, J. L., Brown, M. S., and Shimomura, I. (2001). SREBP cleavage-activating protein (SCAP) is required for increased lipid synthesis in liver induced by cholesterol deprivation and insulin elevation. Genes Dev. 15, 1206-1216.
[51] McAllister, S. D., Soroceanu, L., and Desprez, P.-Y. (2015). The antitumor activity of plant-derived non-psychoactive cannabinoids. J. Neuroimmune Pharmacol. Off. J. Soc. Neurolmmune Pharmacol. 10, 255-267.
[52] McPartland, J. M., Duncan, M., Di Marzo, V., and Pertwee, R. G. (2015). Are cannabidiol and Δ(9)-tetrahydrocannabivarin negative modulators of the endocannabinoid system? A systematic review. Br. J. Pharmacol. 172, 737-753.
[53] Mollinedo, F., and Gajate, C. (2015). Lipid rafts as major platforms for signaling regulation in cancer. Adv. Biol. Regul. 57, 130-146.
[54] Ortegren, U., Karlsson, M., Blazic, N., Blomqvist, M., Nystrom, F. H., Gustaysson, J., Fredman, P., and Stralfors, P. (2004). Lipids and glycosphingolipids in caveolae and surrounding plasma membrane of primary rat adipocytes. Eur. J. Biochem. 271, 2028-2036.
[55] Palmer, A. E., Jin, C., Reed, J. C., and Tsien, R. Y. (2004). Bc1-2-mediated alterations in endoplasmic reticulum Ca2+ analyzed with an improved genetically encoded fluorescent sensor. Proc. Natl. Acad. Sci. U.S.A 101, 17404-17409.
[56] Pan, H., Mukhopadhyay, P., Rajesh, M., Patel, V., Mukhopadhyay, B., Gao, B., Hasid), G., and Pacher, P. (2009). Cannabidiol attenuates cisplatin-induced nephrotoxicity by decreasing oxidative/nitrosative stress, inflammation, and cell death. J. Pharmacol. Exp. Ther. 328, 708-714.
[57] Peng, Y., Myers, R., Zhang, W., and Alexov, E. (2018). Computational Investigation of the Missense Mutations in DHCR7 Gene Associated with Smith-Lemli-Opitz Syndrome. Int. J. Mol. Sci. 19.
[58] Pirmoradi, L., Seyfizadeh, N., Ghavami, S., Zeki, A. A., and Shojaei, S. (2019). Targeting cholesterol metabolism in glioblastoma: a new therapeutic approach in cancer therapy. J. Investig. Med. Off. Publ. Am. Fed. Clin. Res.
[59] Prabhu, A. V., Luu, W., Sharpe, L. J., and Brown, A. J. (2016). Cholesterol-mediated Degradation of 7-Dehydrocholesterol Reductase Switches the Balance from Cholesterol to Vitamin D Synthesis. J. Biol. Chem. 291, 8363-8373.
[60] Qian, Y., Wang, X., and Markowitz, J. S. (2019). Inhibition of Carboxylesterase 1 by Major Cannabinoids and Selected Metabolites. Drug Metab. Dispos. Biol. Fate Chem.
[61] Rajesh, M., Mukhopadhyay, P., Bátkai, S., Patel, V., Saito, K., Matsumoto, S., Kashiwaya, Y., Horváth, B., Mukhopadhyay, B., Becker, L., et al. (2010). Cannabidiol attenuates cardiac dysfunction, oxidative stress, fibrosis, inflammatory and cell death signaling pathways in diabetic cardiomyopathy. J. Am. Coll. Cardiol. 56, 2115-2125.
[62] Ramer, R., and Hinz, B. (2017). Cannabinoids as Anticancer Drugs. Adv. Pharmacol. San Diego Calif 80, 397-436.
[63] Ravier, M. A., Cheng-Xue, R., Palmer, A. E., Henquin, J. C., and Gilon, P. (2010). Subplasmalemmal Ca(2+) measurements in mouse pancreatic beta cells support the existence of an amplifying effect of glucose on insulin secretion. Diabetologia 53, 1947-1957.
[64] Rimmerman, N., Ben-Hail, D., Porat, Z., Juknat, A., Kozela, E., Daniels, M. P., Connelly, P. S., Leishman, E., Bradshaw, H. B., Shoshan-Barmatz, V., et al. (2013). Direct modulation of the outer mitochondrial membrane channel, voltage-dependent anion channel 1 (VDAC1) by cannabidiol: a novel mechanism for cannabinoid-induced cell death. Cell Death Dis. 4, e949.
[65] Rockett, B. D., Teague, H., Harris, M., Melton, M., Williams, J., Wassall, S. R., and Shaikh, S. R. (2012). Fish oil increases raft size and membrane order of B cells accompanied by differential effects on function. J. Lipid Res. 53, 674-685.
[66] San Martín, A., Ceballo, S., Ruminot, I., Lerchundi, R., Frommer, W. B., and Barros, L. F. (2013). A genetically encoded FRET lactate sensor and its use to detect the Warburg effect in single cancer cells. PloS One 8, e57712.
[67] Schmitt, M., Dehay, B., Bezard, E., and Garcia-Ladona, F. J. (2017). U18666A, an activator of sterol regulatory element binding protein pathway, modulates presynaptic dopaminergic phenotype of SH-SYSY neuroblastoma cells. Synap. N. Y. N 71.
[68] Schroeder, R., London, E., and Brown, D. (1994). Interactions between saturated acyl chains confer detergent resistance on lipids and glycosylphosphatidylinositol (GPI)-anchored proteins: GPI-anchored proteins in liposomes and cells show similar behavior. Proc. Natl. Acad. Sci. U.S.A 91, 12130-12134.
[69] Serini, S., Trombino, S., Oliva, F., Piccioni, E., Monego, G., Resci, F., Boninsegna, A., Picci, N., Ranelletti, F. O., and Calviello, G. (2008). Docosahexaenoic acid induces apoptosis in lung cancer cells by increasing MKP-1 and down-regulating p-ERK1/2 and p-p38 expression. Apoptosis Int. J. Program. Cell Death 13, 1172-1183.
[70] Serra, M., and Saba, J. D. (2010). Sphingosine 1-phosphate lyase, a key regulator of sphingosine 1-phosphate signaling and function. Adv. Enzyme Regul. 50, 349-362.
[71] Shaikh, S. R., Dumaual, A. C., Castillo, A., LoCascio, D., Siddiqui, R. A., Stillwell, W., and Wassall, S. R. (2004). Oleic and Docosahexaenoic Acid Differentially Phase Separate from Lipid Raft Molecules: A Comparative NMR, DSC, AFM, and Detergent Extraction Study. Biophys. J. 87, 1752-1766.
[72] Shaikh, S. R., Kinnun, J. J., Leng, X., Williams, J. A., and Wassall, S. R. (2015). How polyunsaturated fatty acids modify molecular organization in membranes: Insight from NMR studies of model systems. Biochim. Biophys. Acta BBA—Biomembr. 1848, 211-219.
[73] Shin, S., Jing, K., Jeong, S., Kim, N., Song, K.-S., Heo, J.-Y., Park, J.-H., Seo, K.-S., Han, J., Park, J.-I., et al. (2013). The omega-3 polyunsaturated fatty acid DHA induces simultaneous apoptosis and autophagy via mitochondrial ROS-mediated Akt-mTOR signaling in prostate cancer cells expressing mutant p53. BioMed Res. Int. 2013, 568671.
[74] Silvius, J. R. (2003). Role of cholesterol in lipid raft formation: lessons from lipid model systems. Biochim. Biophys. Acta BBA—Biomembr. 1610, 174-183.
[75] Smart, E. J., Ying, Y. S., Conrad, P. A., and Anderson, R. G. (1994). Caveolin moves from caveolae to the Golgi apparatus in response to cholesterol oxidation. J. Cell Biol. 127, 1185-1197.
[76] Soni, S. P., LoCascio, D. S., Liu, Y., Williams, J. A., Bittman, R., Stillwell, W., and Wassall, S. R. (2008). Docosahexaenoic Acid Enhances Segregation of Lipids between Raft and Nonraft Domains: 2H-NMR Study. Biophys. J. 95, 203-214.
[77] Staneva, G., Puff, N., Stanimirov, S., Tochev, T., Angelova, M. I., and Seigneuret, M. (2018). The Alzheimer's disease amyloid-β peptide affects the size-dynamics of raft-mimicking Lo domains in GM1-containing lipid bilayers. Soft Matter 14, 9609-9618.
[78] Sun, S.-N., Jia, W.-D., Chen, H., Ma, J.-L., Ge, Y.-S., Yu, J.-H., and Li, J.-S. (2013). Docosahexaenoic acid (DHA) induces apoptosis in human hepatocellular carcinoma cells. Int. J. Clin. Exp. Pathol. 6, 281-289.
[77] Suzuki, K. G. N., Fujiwara, T. K., Sanematsu, F., lino, R., Edidin, M., and Kusumi, A. (2007). GPI-anchored receptor clusters transiently recruit Lyn and G alpha for temporary cluster immobilization and Lyn activation: single-molecule tracking study 1. J. Cell Biol. 177, 717-730.
[78] Takanaga, H., and Frommer, W. B. (2010). Facilitative plasma membrane transporters function during ER transit. FASEB J. Off. Publ. Fed. Am. Soc. Exp. Biol. 24, 2849-2858.
[79] Tsou, P., Zheng, B., Hsu, C.-H., Sasaki, A. T., and Cantley, L. C. (2011). A fluorescent reporter of AMPK activity and cellular energy stress. Cell Metab. 13, 476-486.
[80] Waltl, S., Patankar, J. V., Fauler, G., Nusshold, C., Ullen, A., Eibinger, G., Wintersperger, A., Kratky, D., Malle, E., and Sattler, W. (2013). 25-Hydroxycholesterol regulates cholesterol homeostasis in the murine CATH.a neuronal cell line. Neurosci. Lett. 539, 16-21.
[81] Wang, X., Sato, R., Brown, M. S., Hua, X., and Goldstein, J. L. (1994). SREBP-1, a membrane-bound transcription factor released by sterol-regulated proteolysis. Cell 77, 53-62.
[82] Wassail, S. R., and Stillwell, W. (2008). Docosahexaenoic acid domains: the ultimate non-raft membrane domain. Chem. Phys. Lipids 153, 57-63.
[83] Wassail, S. R., and Stillwell, W. (2009). Polyunsaturated fatty acid-cholesterol interactions: domain formation in membranes. Biochim. Biophys. Acta 1788, 24-32.
[84] Weber, L. W., Boll, M., and Stampfl, A. (2004). Maintaining cholesterol homeostasis: Sterol regulatory element-binding proteins. World J. Gastroenterol. WJG 10, 3081-3087.
[85] Williams, J. A., Batten, S. E., Harris, M., Rockett, B. D., Shaikh, S. R., Stillwell, W., and Wassail, S. R. (2012). Docosahexaenoic and Eicosapentaenoic Acids Segregate Differently between Raft and Nonraft Domains. Biophys. J. 103, 228-237.
[86] Zhou, X., Clister, T. L., Lowry, P. R., Seldin, M. M., Wong, G. W., and Zhang, J. (2015). Dynamic Visualization of mTORC1 Activity in Living Cells. Cell Rep. 10, 1767-1777.
[87] Porter F D, Herman G E. Malformation syndromes caused by disorders of cholesterol synthesis. J. Lipid. Res. 2011; 52:6-34.
[88] Thomson, S., Rice, C. A., Zhang, T., Edrada-Ebel, R., Henriquez, F. L., & Roberts, C. W. (2017). Characterisation of sterol biosynthesis and validation of 14α-demethylase as a drug target in Acanthamoeba. Scientific reports, 7(1), 8247. doi:10.1038/s41598-017-07495-z
[89] Widenmaier, S. B.; Snyder, N. A.; Nguyen, T. B.; Arduini, A.; Lee, G. Y.; Arruda, A. P.; Saksi, J.; Bartelt, A.; Hotamisligil, G. S. NRF1 Is an ER Membrane Sensor that Is Central to Cholesterol Homeostasis. Cell 2017, 171, 1094-1109
[90] U.S. patent application Ser. No. 12/863,842
[91] Hicks D A, Nalivaeva N N, Turner A J. Lipid rafts and Alzheimer's disease: protein-lipid interactions and perturbation of signaling. Front Physiol. 2012; 3:189.
[92] Martin M G, Pfrieger F, Dotti C G. Cholesterol in brain disease: sometimes determinant and frequently implicated. EMBO Rep. 2014; 15(10):1036-1052.
[93] Zhou, Xin et al. “Lipid rafts participate in aberrant degradative autophagic-lysosomal pathway of amyloid-beta peptide in Alzheimer's disease.” Neural regeneration research vol. 9,1 (2014): 92-100.

What is claimed is:

1. A method of modulating the level of cholesterol in a cell comprising the step of:
introducing an effective amount of a cannabidiol (CBD) or a CBD analog to a cell, wherein said effective amount induces one or more of the following:
increasing the lipid order of cholesterol containing cell membranes in said cell;
increasing cellular cholesterol storage in said cell;
altering the orientation of cholesterol present in lipid membranes in said cell;
increasing cellular endoplasmic reticulum cholesterol transport in said cell;
increasing cellular endoplasmic reticulum cholesterol storage in said subject;
activating SREBP-SCAP processing in said cell;
increasing production of HMG-CR in said cell;
inhibiting DHCR24 in said subject sufficient to disrupt cholesterol biosynthesis in said cell; and
inhibiting DHCR7 sufficient to disrupt cholesterol biosynthesis in said cell.
and wherein said effective amount does not induce apoptosis in said cell.

2. The method of claim 1 wherein said cell is a human cell.

3. The method of claim 2 wherein said step of introducing an effective amount of a CBD or a CBD analog to a cell comprises the step of introducing an effective amount of a CBD or a CBD analog to a cell in vivo, in vitro or ex vivo.

4. The method of claim 1 wherein said CBD or a CBD analog is isolated from a Cannabis plant.

5-7. (canceled)

8. The method of claim 1 wherein said CBD analog comprises a CBD analog selected from the group consisting of: HU-308, o-1821, o-1602, abnormal CBD, ajulemic Acid, DHM-CBD, deoxy0CBD, 11-hydroxy-CBD, CBD-11-oic acid; CP 55,940, HU-210, HU-211, HU331, and/or 11-hydroxy-Δ9-THC.

9. A method of modulating the level of cholesterol in a subject comprising the step of:
administering a therapeutically effective amount of cannabidiol (CBD) or a CBD analog to a subject, wherein said subject is suffering from, or predisposed to developing a cholesterol-related condition and wherein said therapeutically effective amount induces one or more of the following:
increasing the lipid order of cholesterol containing cell membranes in said subject;
increasing cellular cholesterol storage in said subject;
altering the orientation of cholesterol present in lipid membranes in said subject;
increasing cellular endoplasmic reticulum cholesterol transport in said subject;
increasing cellular endoplasmic reticulum cholesterol storage in said subject;
activating SREBP-SCAP processing in said subject;
increasing production of HMG-CR in said cell;
inhibiting DHCR24 in said subject sufficient to disrupt cholesterol biosynthesis in said subject; and
inhibiting DHCR7 sufficient to disrupt cholesterol biosynthesis in said subject.
and wherein said therapeutically effective amount does not induce cell apoptosis.

10. The method of claim 9 wherein said cell is a human cell.

11. The method of claim 10 wherein said step of introducing an effective amount of a CBD or a CBD analog to a cell comprises the step of introducing an effective amount of a CBD or a CBD analog to a cell in vivo, in vitro or ex vivo.

12. The method of claim 9 wherein said CBD or a CBD analog is isolated from a Cannabis plant.

13-15. (canceled)

16. The method of claim 9 wherein said CBD analog comprises a CBD analog selected from the group consisting of: HU-308, o-1821, o-1602, abnormal CBD, ajulemic Acid, DHM-CBD, deoxy0CBD, 11-hydroxy-CBD, CBD-11-oic acid; CP 55,940, HU-210, HU-211, HU331, and/or 11-hydroxy-Δ9-THC.

17-28. (canceled)

29. A method of treating a subject having a lipid-order disease condition comprising the step of administering a therapeutically effective amount of cannabidiol (CBD) or a CBD analog, wherein said therapeutically effective amount increases the lipid order of cholesterol containing cell membranes in said subject.

30. The method of claim 29 wherein said lipid-order disease comprises Alzheimer's disease.

31. The method of claim 30 wherein said subject is a mammal.

32. The method of claim 32 wherein said mammal is a human.

33. The method of claim 29 wherein said therapeutically effective amount does not increase levels of cellular apoptosis.

34. The method of claim 29 wherein said CBD analog is selected from the group consisting of: wherein said CBD analog comprises a CBD analog selected from the group consisting of: HU-308, o-1821, o-1602, abnormal CBD, ajulemic Acid, DHM-CBD, deoxy0CBD, 11-hydroxy-CBD, CBD-11-oic acid; CP 55,940, HU-210, HU-211, HU331, and/or 11-hydroxy-Δ9-THC

35. The method of claim 29 wherein said CBD or a CBD analog is isolated from a Cannabis plant.

36. The method of claim 29 wherein said CBD or a CBD analog is synthetically manufactured.

37. The method of claim 29 and further comprising the step of administrating a therapeutically effective amount of a lipid-ordering compound.

38. The method of claim 30 and further comprising the step of co-administrating a therapeutically effective amount of an Alzheimer's disease controlling compound selected from the group consisting of: a cholinesterase inhibitor; a NMDA inhibitor, donepezil, memantine, donepezil, galantamine, and rivastigmine.

39-83. (canceled)
